(19) United States
(12) Patent Application Publication
(10) Pub. No.: US 2026/0100252 A1
(43) Pub. Date: Apr. 9, 2026

(54) ADVANCED RETROSYNTHESIS-RELATED SYNTHETIC ACCESSIBILITY MODELING

(71) Applicants: Insilico Medicine IP Limited, Hong Kong (HK); Insilico Medicine AI Limited, Abu Dhabi (AE)

(72) Inventors: Bogdan Zagribelnyy, Abu Dhabi (AE); Sergei Fedorchenko, Abu Dhabi (AE); Nikita Bondarev, Abu Dhabi (AE); Ivan Ilin, Abu Dhabi (AE); Yan Ivanenkov, Abu Dhabi (AE); Aleksandrs Zavoronkovs, Hong Kong (HK)

(21) Appl. No.: 19/353,038

(22) Filed: Oct. 8, 2025

Related U.S. Application Data
(60) Provisional application No. 63/705,256, filed on Oct. 9, 2024.

(51) Int. Cl. G16C 20/10; G16C 20/20; G16C 20/70; G16C 20/90

(52) U.S. Cl. CPC G16C 20/10; G16C 20/20; G16C 20/70; G16C 20/90

(57) ABSTRACT

A method for training a model to estimate synthetic accessibility may be provided. A retrosynthesis-related synthetic accessibility model may be provided. A molecular structures database may be accessed. At least one molecular structure may be obtained from the molecular structures database with the model. The at least one molecular structure may be virtually sliced into synthon-like fragments with the model. A frequency of the synthon-like fragments in natural molecules may be determined with the model. Molecular descriptors for the synthon-like fragments may be calculated with the model. An aggregated synthetic accessibility score for the synthon-like fragments may be determined with the model. The aggregated synthetic accessibility score for the synthon-like fragments may be stored in a database for the model.

### CROSS-REFERENCE TO RELATED APPLICATIONS

This patent application claims priority to U.S. Provisional Application No. 63/705,256 filed Oct. 9, 2024, which provisional is incorporated herein by specific reference in its entirety.

### BACKGROUND

### Field

The present disclosure relates to computing systems that perform computations for use as synthetic accessibility prediction in computational chemistry and drug discovery applications. More specifically, the disclosure relates to computer-implemented methods and systems for estimating synthetic feasibility of molecular structures using retrosynthesis-related analysis and machine learning techniques.

### Description of Related Art

Recent developments in generative chemistry have significantly advanced both de novo molecular design and optimization efforts, including hit-to-lead and lead optimization processes. The integration of artificial intelligence and machine learning tools has facilitated the rapid production of thousands of molecular structures tailored to exhibit specific predicted characteristics, such as physico-chemical, biological activity, and ADME properties (absorption, distribution, metabolism, excretion). While the computational prediction of these properties is a critical initial filter for potential candidates, estimating the synthetic feasibility of candidate compounds is essential for determining which structures should progress to laboratory synthesis.

Unlike traditional drug design, which typically yields fewer candidate structures, AI-driven drug discovery produces a much larger pool of virtual candidates, increasing the importance of robust synthetic accessibility (SA) models. Proper SA models help predict the practical synthesizability of a molecular structure, incorporating aspects of synthetic chemistry logic, medicinal chemistry experience, and market considerations. The use of more accurate SA models results in synthetic feasibility assessments that better reflect actual laboratory outcomes, thereby improving the efficiency with which virtual candidates are translated into synthesized compounds.

Synthetic accessibility is generally represented by a score assigned to each compound, which assists researchers in prioritizing synthesis efforts, reducing costs, and managing project timelines while achieving targeted hit rates. It is important to note that there is currently no universally accepted definition of synthetic accessibility; as a result, pharmaceutical and biotechnology companies often develop proprietary computational methods for SA determination. These methods may account for various factors, including the number and complexity of substructures present in a molecule, the availability of building blocks and reagents within in-house and commercial databases, the economics of synthesis, and the number of steps required in the proposed synthetic route. The assignment of an SA score enables more effective decision-making during chemical synthesis planning and resource allocation.

Retrosynthetic planning tools require extensive computational resources and processing time. These tools analyze molecular structures through complex algorithmic pathways. The computational burden limits their application in high-throughput screening scenarios.

Statistical approaches have been developed to address computational efficiency concerns. These methods rely on molecular descriptors and complexity metrics. The descriptors often fail to correlate accurately with actual synthetic feasibility. Simple molecular complexity does not necessarily indicate synthetic difficulty.

Data-driven machine learning models have emerged as alternative solutions. These models require large training datasets of synthesized compounds. Dataset completeness remains a persistent challenge in the field. Labeling accuracy of training data affects model performance significantly.

Generative artificial intelligence systems produce large numbers of molecular candidates. These systems generate thousands of potential drug compounds rapidly. Current synthetic accessibility prediction methods cannot process such volumes efficiently. The speed limitations create bottlenecks in AI-driven drug discovery workflows.

Existing synthetic accessibility scores often produce inconsistent results for similar molecules. Small structural changes can lead to dramatically different accessibility predictions. This phenomenon creates challenges for structure-activity relationship analysis. Medicinal chemists struggle to interpret conflicting accessibility assessments.

Multiple synthetic routes may exist for individual target molecules. Current methods typically evaluate single retrosynthetic pathways. The failure to consider alternative synthetic approaches limits prediction accuracy. Route diversity information would provide more comprehensive accessibility assessments.

Reaction feasibility varies significantly across different chemical transformations. Some reaction types are well-established and reliable in synthetic laboratories. Other transformations present significant synthetic challenges or require specialized conditions. Current accessibility models inadequately distinguish between different reaction difficulties.

### SUMMARY

In some embodiments, a method for training a model to estimate synthetic accessibility can include: providing a retrosynthesis-related synthetic accessibility model; accessing a molecular structures database; obtaining at least one molecular structure from the molecular structures database with the model; virtually slicing the at least one molecular structure into synthon-like fragments with the model; determining a frequency of the synthon-like fragments in natural molecules with the model; calculating molecular descriptors for the synthon-like fragments with the model; determining an aggregated synthetic accessibility score for the synthon-like fragments with the model; and storing the aggregated synthetic accessibility score for the synthon-like fragments in a database for the model.

In some embodiments, a method for training a model to assess synthetic accessibility involves first gathering information about chemical compounds whose synthetic outcomes are known, such as compounds that have been synthesized in the laboratory or are commercially available. Each compound is analyzed to identify retrosynthetic fragments and to calculate structural descriptors that capture its complexity. The frequency with which each fragment appears in reference databases and supplier catalogs is determined and recorded. These data points, such as fragment occurrences, availability, and structural characteristics, are used to create a training dataset for a machine learning algorithm. The algorithm is then trained to recognize the patterns that distinguish compounds that are easily synthesized from those that are more challenging. Once trained, the model is able to predict the synthetic accessibility of new compounds based on their structural features and fragment profiles.

In some embodiments, a subscore of each synthon-like fragment is calculated with the model as a function taking into account descriptors and fragment frequency in a dictionary over the whole training dataset. In some aspects, the subscore represents a chemical complexity of the synthon-like fragment, including: number of chiral carbon atom, total number of rings, number of side chains attached to the ring systems, number of spiro carbon atoms, number of atoms in the largest ring of molecular structure if it is bigger than 6, otherwise 0, number of fused rings in a molecular structure, number of bridgehead atoms in the bicyclic pattern(s) of molecular structure, number of atoms other than hydrogen, molecular weight value, and normalized quadratic index 1 calculated as (3−2·A+Z1/2), where A is the number of heavy atoms and Z1 is the first Zagreb index.

In some embodiments, the training method can include: performing a canonicalization for normalizing a chemical line notation of the molecular structure, where the valence, charge, and aromaticity is checked; inputting the molecular structure if the valence, charge, and aromaticity are valid; and removing the molecular structure if the valence, charge, and/or aromaticity are invalid.

In some embodiments, the training method can include: performing a decomposition on the molecular structure into a set of splits; checking available of fragment in a fragment dictionary that has the fragments and frequency thereof; obtaining primary split score from all subscores; obtaining transformed split score with a mathematic transformation; obtaining final split score from the transformed split score taking into account number of fragments that are commercially available building blocks, where more commercially available building blocks for the fragments has a lower final split score; and aggregating final split scores for the fragments taking into account differences between a split with lower final split score and other final split scores of other splits to obtain a retrosynthesis-related synthetic accessibility model score.

In some embodiments, the training method can include smoothing retrosynthesis-related synthetic accessibility model scores for a plurality of molecular structures to avoid sharp drops in scores over a chemical space of the plurality of molecular structures.

In some embodiments, the training method can include training the model with the retrosynthesis-related synthetic accessibility model scores for a plurality of molecular structures.

In some embodiments, a method of estimating synthetic accessibility: inputting a target molecule into a synthetic accessibility system having a retrosynthesis-related synthetic accessibility model; decomposing the target molecule into molecular synthon-like fragments; converting the synthon-like fragments into their synthetic equivalents; indexing the synthetic equivalents in the dataset of commercially available starting materials; providing penalties for synthetically irrelevant unprecedented substructures; calculating a synthetic accessibility score components for the molecular fragments for the target molecular structure; determining a sum of synthetic accessibility score components for the fragments; and providing the synthetic accessibility score and building blocks visualization for the target molecule.

In some embodiments, a method of estimating synthetic accessibility: inputting a target molecule into a synthetic accessibility system having a retrosynthesis-related synthetic accessibility model; decomposing the target molecule into molecular synthon-like fragments; converting the synthon-like fragments into their synthetic equivalents; indexing the synthetic equivalents in the dataset of commercially available starting materials; providing penalties for synthetically irrelevant unprecedented substructures; calculating a synthetic accessibility score components for the molecular fragments for the target molecular structure; determining a sum of synthetic accessibility score components for the fragments; and providing the synthetic accessibility score and building blocks visualization for the target molecule.

In some embodiments, a method for estimating synthetic accessibility may include receiving a target molecule as input to a synthetic accessibility system that employs a retrosynthesis-related synthetic accessibility model. The system may decompose the target molecule into synthon-like molecular fragments and convert these fragments into their corresponding synthetic equivalents. The synthetic equivalents may then be matched or indexed against a dataset containing commercially available starting materials. The method may apply penalties for molecular substructures that are synthetically irrelevant or not previously observed. For each of the molecular fragments, the system may calculate components of a synthetic accessibility score, and determine an overall score by summing these components. The synthetic accessibility score, along with a visualization of relevant building blocks, may then be provided for the target molecule.

In some embodiments, the method can include determining frequency of synthon-like fragments by: obtaining a retrosynthetic decomposition of the molecular structure of the target molecule to obtain synthon-like fragments thereof; organizing the synthon-like fragments into sets of fragments; aggregating sets of fragments into a homogenous dataset of synthon-like fragments; calculating frequencies of the synthon-like fragments in a reported chemical space, such as natural or non-synthesized molecules; obtaining a dataset of the synthon-like fragments associated with their respective calculated frequencies; and identifying synthon-like fragments with higher frequencies.

In some embodiments, the method can include: determining a synthetic accessibility of the molecular structure where synthon-like fragments with higher frequencies have a higher contribution to the synthetic accessibility; and providing the synthetic accessibility for a synthetic route of the target molecule.

In some embodiments, the method can include performing a target molecular structure assessment for synthetic accessibility by: obtaining a retrosynthetic decomposition of the molecular structure of the target molecule to obtain synthon-like fragments thereof; organizing the synthon-like fragments into sets of fragments that are synthetic equivalents; indexing the synthetic equivalent sets of fragments; identifying commercially available starting materials for the synthetic equivalent sets of fragments; and obtaining a dataset of the indexed synthetic equivalent sets of fragments associated with the commercially available starting materials, wherein when more synthetic equivalents are found in the dataset the higher contribution to the final estimate of synthetic accessibility is obtained for the molecular structure.

In some embodiments, the method can include: taking into account the context of molecular complexity for synthon-like fragments, wherein the molecular structure is described with structural descriptors; and storing descriptor values for each synthon-like fragment in a database for the retrosynthesis-related synthetic accessibility model.

In some embodiments, the method can include filtering molecular structures by: analyzing fragments of synthon-like fragments in the set of fragments; and rewarding fragments that possessing multiple synthetic routes, wherein more diverse sets of fragments with higher number of synthetic routes have a higher contribution to determined synthetic accessibility.

In some embodiments, the method can include analyzing a synthetic route of a target molecule by: obtaining a virtual reaction for a reaction step in the synthetic route; analyzing the virtual reaction for influence on synthetic accessibility of the target molecule; rewarding multi-component reaction steps; penalizing macrocyclization reactions; obtaining a predetermined synthetically irrelevant substructures inadequate for a reaction step; penalizing a synthesis route with the predetermined synthetically irrelevant substructures; and determining synthetic accessibility for the target molecule after the rewards and/or penalties.

In some embodiments, the method can include calculating an overall retrosynthesis-related synthetic accessibility score by: obtaining score of frequencies of synthon-like fragments; obtaining score of building blocks of target molecule; obtaining score of structural descriptor values; obtaining score of diversity and/or similarity score of sets of fragments; obtaining score of rewards and/or penalties for reaction step of synthetic route; obtaining score of penalty for synthetically irrelevant synthon-like fragment or portion thereof; and aggregating the score of each of the foregoing to obtain a final synthetic accessibility score.

In some embodiments, the method can include: identifying a first target molecule with a lower synthetic accessibility score compared to a second target molecule; selecting the first target molecule for synthesis; and obtaining the synthetic route with the lower synthetic accessibility score.

In some embodiments, the method can include: synthesizing the target molecule to obtain a real, physical form of the target molecule.

In some embodiments, the method can include obtaining real, physical forms of starting reagents for the synthetic route of the first targe molecule.

In some embodiments, one or more non-transitory computer readable media storing instructions that in response to being executed by one or more processors, cause a computer system to perform operations, the operations comprising the methods described herein.

In some embodiments, a computer system can include: one or more processors; and one or more non-transitory computer readable media storing instructions that in response to being executed by the one or more processors, cause the computer system to perform operations, the operations comprising the methods described herein.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 includes a schematic representation of the ReRSA scoring algorithm.

FIG. 2A illustrates Algorithm 1.

FIG. 2B illustrates Algorithm 2.

FIG. 2C illustrates Algorithm 3.

FIG. 2D illustrates Algorithm 4.

FIG. 2E illustrates Algorithm 5.

FIG. 2F illustrates Algorithm 6.

FIG. 2G illustrates Algorithm 7.

FIG. 2H illustrates Algorithm 8.

FIGS. 3A-3F illustrate decomposition of the dummy molecular structure into splits.

FIGS. 4A-4C show the decomposition of sacubitril where only the splits with the highest scores are provided.

FIGS. 5A-5C show the decomposition of baricitinib where only the splits with the highest scores are provided.

FIGS. 6A-6B illustrate two samples from the generative chemistry platform Chemistry42 assessed for synthetic accessibility and distinguished within ReRSA score.

FIG. 7A includes visualization of ReRSA output from Chemistry42 for danoprevir (partially converted drug).

FIG. 7B includes visualization of ReRSA output from Chemistry42 for lifitegrast (partially converted drug).

FIG. 7C includes visualization of ReRSA output from Chemistry42 for maraviroc (fully converted drug).

FIG. 7D includes visualization of ReRSA output from Chemistry42 for ivosidenib (fully converted drug).

FIGS. 8A-8B include visualization of ReRSA output from Chemistry42 for two partially converted generated structures.

FIGS. 9A-9B include visualization of ReRSA output from Chemistry42 for two fully converted generated structures.

FIG. 10 illustrates examples for structures accessed by ReRSA in both SOFT and STRICT policies.

FIGS. 11A-11F illustrate the comparison of discrimination power of SA modeling approaches on T_{CP} SYBA test dataset.

FIG. 12 illustrates randomly selected structures from T_{CP-} set.

FIG. 13 includes a graph that illustrates the counts of drugs and counts of generated structure for easy synthesis (ES) and hard synthesis (HS).

FIGS. 14A-14G includes bar charts showing the discrimination power of different SA modeling methods.

FIG. 15 shows an example computing device (e.g., a computer) that may be arranged in some embodiments to perform the methods (or portions thereof) described herein.

FIGS. 16A-16H illustrate ReRSA exploited robust reactions schemes.

FIGS. 17A-17F show examples of compounds ordered by ReRSA 3.0 score with SOFT policy used.

FIGS. 18A-18F show examples of compounds ordered by ReRSA 3.0 score with STRICT policy used.

FIG. 19 illustrates local alterations in a single chemotype are reflected in the corresponding ReRSA scores, with alterations of ReRSA 3.0 scores reflecting local alterations for a single chemotype.

FIG. 20A includes an example of incorrect SMILES-strings provided in the T_{CP} set.

FIG. 20B includes an example of incorrect SMILES-strings provided in the T_{CP} set.

FIG. 21 includes an example of non-druglike molecule included in T_{CP+} set.

FIGS. 22A-22S shows the final dataset with calculated scores and consensus expert classifications.

The elements and components in the figures can be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present technology is related to a system with a computing device configured to execute synthetic accessibility modeling operations. The computing device may include one or more processors operatively coupled to memory storage components. The memory storage components may store executable instructions that, when executed by the processors, may cause the computing device to perform the synthetic accessibility estimation methods described herein.

The computing device may comprise a fragment decomposition engine configured to process molecular structures. The fragment decomposition engine may receive molecular structure input in SMILES string format. The engine may apply retrosynthetic logic to decompose the molecular structure into synthon-like fragments. The decomposition may utilize a library of robust chemical reactions encoded as SMARTS strings. Each robust reaction may represent widely-used synthetic transformations in organic chemistry practice.

The system may include a statistical analysis module operatively connected to the fragment decomposition engine. The statistical analysis module may calculate frequencies of synthon-like fragments within reference chemical databases. The reference databases may comprise commercially available compound libraries such as ChEMBL, PubChem, or vendor stock databases. The statistical analysis module may determine fragment occurrence rates across the reference chemical space. Higher fragment frequencies may correlate with increased synthetic accessibility of structures containing those fragments.

A building block conversion module may be integrated within the computing device architecture. The building block conversion module may transform synthon-like fragments into commercially available building blocks. The conversion may utilize predefined transformation rules that map fragment structures to real chemical compounds. The building block conversion module may query vendor databases to verify commercial availability of identified building blocks. Available building blocks may contribute positively to synthetic accessibility scoring.

The computing device may incorporate a molecular descriptor calculation engine. The molecular descriptor calculation engine may analyze structural features of both fragments and complete molecular structures. The engine may calculate descriptors including chiral center counts, ring system complexity, spiro point presence, and molecular weight parameters. Each descriptor may contribute to an overall complexity assessment that influences synthetic accessibility scoring.

A split analysis component may operate within the system architecture. The split analysis component may organize synthon-like fragments into discrete sets termed splits. Each split may represent an independent retrosynthetic pathway for the target molecule. The component may evaluate similarities between different splits using molecular fingerprint comparisons. Diverse splits may receive higher scoring contributions than similar splits.

The system may include a reaction type evaluation module. The reaction type evaluation module may analyze virtual reactions applied during molecular decomposition. The module may assign positive scoring contributions to multi-component reactions. Macrocyclization reactions may receive negative scoring penalties. The reaction type evaluation may influence the final synthetic accessibility assessment.

A substructure penalty engine may be incorporated into the computing device. The substructure penalty engine may maintain a library of synthetically irrelevant substructures encoded as SMARTS patterns. The engine may scan target molecules for matches against the penalty library. Identified irrelevant substructures may result in scoring penalties that decrease synthetic accessibility estimates.

The computing device may implement a policy selection interface. The policy selection interface may allow users to choose between STRICT and SOFT scoring policies. The STRICT policy may filter structures containing non-represented fragments or substructures. The SOFT policy may apply penalties rather than complete filtration for such structures. The policy selection may balance novelty considerations against synthetic accessibility requirements.

A score aggregation processor may combine multiple scoring components into unified synthetic accessibility values. The score aggregation processor may weight individual components according to predetermined algorithms. Fragment statistics, building block availability, molecular descriptors, split diversity, reaction rewards, and substructure penalties may all contribute to final scores. The score aggregation processor may normalize scores to a standardized scale ranging from 1 to 10.

The system may incorporate a visualization generation module. The visualization generation module may create graphical representations of molecular structures with identified building blocks highlighted. The module may overlay commercial availability information including vendor details and CAS registry numbers. Interactive molecular viewers may allow users to explore different synthetic pathway options.

A database management system may support the computing device operations. The database management system may maintain indexed collections of fragment frequencies, building block libraries, and reference chemical structures. The system may optimize query performance for real-time synthetic accessibility scoring requests. Periodic updates to reference datasets may ensure scoring accuracy reflects current commercial availability. Therefore, the datasets used in the methods described herein are based on real molecules, such those in databases and commercially available.

The computing device may include network communication interfaces for external system integration. The interfaces may support API connections to generative chemistry platforms and synthesis planning tools. Batch processing capabilities may enable evaluation of multiple molecular structures simultaneously. The communication interfaces may facilitate integration with existing drug discovery workflows.

A user interface component may provide access to synthetic accessibility scoring functionality. The user interface may accept molecular structure inputs through various formats including SMILES strings and molecular drawing tools. Results presentation may include numerical scores, detailed breakdowns, and visual building block identification. Export capabilities may allow results integration with external analysis tools.

The system architecture may support distributed computing implementations. Multiple computing devices may operate in parallel to process large molecular datasets. Load balancing mechanisms may distribute computational tasks across available processors. Scalable storage systems may accommodate growing reference databases and fragment libraries.

Quality control modules may validate molecular structure inputs and scoring outputs. Input validation may verify chemical structure validity and canonical representation. Output validation may ensure scoring consistency and identify potential calculation errors. Error handling mechanisms may provide appropriate responses to invalid inputs or system failures.

The computing device may implement caching mechanisms to improve performance. Frequently accessed fragment statistics and building block information may be stored in high-speed memory. Caching may reduce database query times and accelerate scoring operations. Cache management algorithms may balance memory usage with performance optimization.

In some embodiments, a method for training a model to estimate synthetic feasibility may comprise multiple steps. In a first step, the method may include accessing a molecular structures database to obtain relevant data. In a second step, the method may involve virtually slicing the molecular structures into synthon-like fragments using retrosynthetic analysis. In a third step, the method may determine a frequency of occurrence for the generated synthon-like fragments within the dataset. In a fourth step, the method may calculate molecular descriptors for these fragments to characterize their complexity and other relevant features. In a final step, the method may store the aggregated synthetic accessibility score components for the reported synthon-like fragments in a database for future reference or model training.

In some embodiments, a method for converting synthon-like fragments into synthetic accessibility scores may comprise several steps. In a first step, the method may select a synthon-like fragment generated during retrosynthetic analysis. In a second step, the method may access stored frequency information for the selected fragment within a larger molecular structures database. In a third step, the method may retrieve molecular descriptors related to the complexity, size, or other properties of the fragment. In a fourth step, the method may input the frequency information and molecular descriptors into a predefined function or machine learning model configured to output a synthetic accessibility score. In a final step, the method may generate and store the resulting synthetic accessibility score, which can then be used in further retrosynthesis planning or compound evaluation processes.

In some embodiments, the subject matter herein can be supplemented with subject matter from WO2021229454A1, which is incorporated herein by specific reference in its entirety.

In some embodiments, to define synthetic accessibility (SA) properly, a first step can be to define the concept of synthetic feasibility (SF). Synthetic feasibility may be characterized as a property of a molecule that can be assigned a value of synthesizable (1) or not synthesizable (0). In some embodiments, SF may be interpreted as a Bernoulli random variable with two possible outcomes: synthesizable (1) or not synthesizable (0). The determination of SF may consider all current synthetic knowledge and technologies, as well as all possible synthetic plans, available starting materials, costs, labor, and time that may be allocated for a synthetic campaign to produce the molecule as a synthesized compound. Thus, SF (0 or 1) may serve as a binary outcome reflecting the feasibility of synthesis under existing conditions. Synthetic accessibility (SA) may then be defined as the probability, denoted as p, of achieving the successful outcome (1) for the Bernoulli random variable representing SF. As such, SA represents the likelihood or probability that a given molecule can be synthesized, based on the aforementioned factors and current synthetic capabilities.

$$SF ( ω ) = \{ \begin{matrix}0 , & a compound is not synthesizable ; \\ 1 , & a compound is synthesizable\end{matrix} .$$

$${F}_{SF} ( x ) = \{ \begin{matrix}0 , & if x < 0 ; \\ 1 - SA , & if 0 \leq x < 1 ; \\ 1 , & if x \geq 1\end{matrix} .$$

The complement of SA, calculated as

$$q = 1 - p = 1 - SA ,$$

The forgoing equation represents the probability of a compound not being synthesizable under current conditions (probability of failure, 0). This accounts for the inherent uncertainty and limitations in synthetic chemistry, reflecting the chance that, despite the theoretical accessibility, practical synthesis may not be achievable.

Since SA is defined as a Bernoullian probability (p) all properties and consequences of probability are true for SA including the law of total probability:

$$SA = {\sum }_{i = 1}^{n} P ( SF = 1 \vert {C}_{i} ) \cdot P ( {C}_{i} ) ,$$

$${SA}_{i} = P ( SF = 1 \vert {C}_{i} ) ,$$

The foregoing equations account for multiple different countable (n) sets of conditions (Ci) in which condition-associated SAi values can be calculated. Such conditions are different available sets of starting materials, synthetic plans to be assessed, synthetic methods and technologies available at a lab, labor, budget and time that can be allocated to the synthetic campaign etc.

### SA Modeling Approaches

The modem understanding of SA modeling can be conditionally represented by three approaches (groups of approaches).

The retrosynthetic approach may represent a classical method for estimating the synthesizability of a molecule. This approach may involve determining the sequence of chemical reactions needed to obtain a target molecular structure from commercially available starting materials. Synthesizability may be indicated using a binary metric. A value of one may correspond to identifying a viable retrosynthetic pathway, suggesting the molecular structure is synthetically feasible. Conversely, a value of zero may indicate that no retrosynthetic pathway was found, suggesting the molecular structure is estimated to be synthetically unfeasible.

A molecular descriptor may be a characteristic of a molecular structure. For example, a molecular descriptor can be the molecular weight or the count of chiral carbon atoms. The molecular descriptor can be associated directly or indirectly with synthetic complexity. In contrast, a reaction descriptor may be a characteristic of a molecular reaction or a sequence of reactions. For example, a reaction descriptor can indicate whether macrocyclization is involved, or represent the number of reaction steps.

The data-driven approach can be divided into statistics-based and machine learning-driven subcategories. The statistics-based approach may rely on reported chemical space to generate statistical insights on synthetic accessibility. The machine learning-driven approaches are often implemented as supervised learning methods. These approaches require a labeled dataset, such as a binary labeled dataset that classifies molecular structures as easy-to-synthesize or hard-to-synthesize.

### Statistics-Based SA Modeling

In some embodiments, statistics-based approaches to modeling synthetic accessibility may utilize the analysis of molecular structures by decomposing these structures into motifs or fragments that are prevalent within representative chemical databases, such as those containing commercially available pharmaceutical compounds or synthesized chemical entities. A frequency metric may be assigned to each identified motif, where a motif that appears more frequently within the sample chemical space may be associated with an increased degree of synthetic accessibility for molecules containing that motif. The statistical approach may operate under the hypothesis that the synthetic accessibility of a candidate molecule is positively correlated with the aggregate frequencies of its structural motifs within the reference dataset. In particular, the process may comprise parsing a molecular structure into its constituent substructures, calculating the occurrence rate of each substructure in one or more reference chemical databases, and calculating an accessibility score for the candidate molecule as a function of these rates. The higher the aggregate frequency of the motifs present within a given molecular structure, the greater the probability that the molecule may be synthesized under standard laboratory conditions using known synthetic methods and available starting materials. In this manner, the statistical analysis provides a quantitative framework for assessing and ranking the synthetic feasibility of compounds in silico, thereby facilitating the selection of molecules for further investigation or synthesis planning.

In some embodiments, a synthetic accessibility metric may be computed using an algorithm such as the Synthetic Accessibility Score (SA Score), which may be implemented using cheminformatics toolkits, for example, the RDKit library. The SA Score may utilize a hybrid methodology that combines molecular descriptor analysis with statistical evaluation of synthetic feasibility based on established chemical synthesis knowledge. In certain implementations, the statistical component of the SA Score may analyze the frequency of occurrence of molecular fragments, where a fragment may refer to a substructure of a molecule generated by applying a fingerprinting method, such as the extended-connectivity fingerprint (for example, ECFC_4), to fragment a molecule into constituent substructures. The occurrences of these fragments may be compared against one or more curated databases of previously synthesized molecules, such as a subset of the PubChem repository.

Additionally, the SA Score may incorporate a penalty term that evaluates the molecular complexity of the candidate structure. This penalty may be assigned based on structural features that are known to increase synthetic difficulty. For example, the presence of multiple chiral centers, a high number of spiro connections, or the occurrence of macrocyclic rings may each result in an increased penalty score for the molecule. These complexity penalties may be computed using predefined criteria or rulesets encoded within the algorithm. As a result, the SA Score provides an aggregate value that represents a trade-off between the rapid estimation achievable through structural complexity assessment and the more detailed, resource-intensive analysis obtained via retrosynthetic pathway modeling. This approach enables efficient large-scale screening of candidate molecules for synthetic tractability within computational workflows.

### ML-Driven SA Modeling

In some embodiments, the ML-driven SA models SCScore and SYBA can be used for benchmarking reasons. RA Score utilizes an approach different from purely ML-driven SCScore and SYBA SA modeling approaches.

In some embodiments, a synthetic accessibility evaluation module may implement a data-driven approach for assessing the synthetic complexity of molecular compounds using a Synthetic Complexity Score (SCScore). The SCScore may be calculated by a fully-connected artificial neural network (ANN) that is trained utilizing a standard backpropagation algorithm on a database comprising a plurality of drug-like molecules along with their associated synthetic routes. The ANN may be configured to learn a function that estimates the synthetic complexity based on precedent chemical reaction data. For a given chemical reaction, the SCScore ranking function may be formulated such that the product of the reaction is associated with a greater SCScore relative to each of the individual reactants. This approach may be effective for assessing complexity at the start or end of a synthetic sequence. However, within multi-step retrosynthetic pathways, this “product-greater-than-reactant” assumption may not consistently hold because, in intermediate transformations, the distinction between product and reactant complexity may blur. Furthermore, strategic synthetic operations, such as the introduction of protecting groups, may locally reduce synthetic complexity, rendering an intermediate compound more reactive or synthetically accessible. As a result, embodiments may recognize that an effective synthetic complexity metric should exhibit a monotonically decreasing trend from a target molecule to its precursor materials over the entire retrosynthetic sequence. The SCScore-based module may be configured to leverage these characteristics for improved retrosynthetic analysis and synthetic pathway planning.

In some embodiments, the SYnthetic Bayesian Accessibility (SYBA) model may be utilized to assess the synthetic accessibility of organic compounds by rapidly classifying candidate molecules as easy-to-synthesize or hard-to-synthesize. The SYBA model can operate as a fragment-based synthetic accessibility scoring tool that employs a Bernoulli naive Bayes classifier to assign contribution scores to individual molecular fragments. These contributions may be determined on the basis of fragment frequencies observed in curated datasets of easy- and hard-to-synthesize compounds. To employ the SYBA model within a retrosynthetic or compound evaluation workflow, a molecular structure may first be decomposed into constituent fragments using predefined cheminformatics algorithms such as fingerprinting or fragmentation schemes. Each fragment may be compared to reference datasets to determine its frequency within the classes of easy- and hard-to-synthesize molecules.

The SYBA model may calculate a synthetic accessibility score by aggregating the contributions of all identified fragments in the candidate molecule. A higher prevalence of fragments associated with easy-to-synthesize reference compounds may increase the resulting synthetic accessibility score, indicating a greater likelihood that the target structure can be synthesized using standard laboratory techniques. Conversely, the presence of rare or difficult-to-synthesize fragments may lower the score and suggest greater synthetic complexity. The SYBA score may thus facilitate the prioritization of candidate molecules in computational or experimental chemistry workflows by providing a rapid, quantitative estimate of synthetic tractability.

This approach may be integrated into automated compound selection, generative chemistry pipelines, or retrosynthesis planning software to enable real-time filtering or ranking of designed molecules according to their predicted accessibility. By enabling early identification of synthetically tractable compounds, the SYBA model may support more efficient resource allocation during compound design, library enumeration, and synthetic route planning processes.

In some embodiments, the Retrosynthetic Accessibility score (RAscore) may be employed to evaluate the synthetic accessibility of candidate molecules within a computational chemistry workflow. The RAscore is a machine learning-based classifier trained on outcomes from computer-aided synthesis planning platforms, such as AiZynthFinder. The RAscore integrates both retrosynthesis-derived data and machine learning-driven analysis, thereby combining information regarding the feasibility of retrosynthetic routes with the predictive capabilities of classification algorithms.

To apply the RAscore in practical use, a molecular structure may first be represented using an extended-connectivity fingerprint encoding structural features relevant to retrosynthesis. This fingerprint representation may be provided as input to a machine learning classifier—such as a neural network, random forest, or XGBoost model—that has been previously trained to distinguish between molecules classified as accessible or inaccessible by automated retrosynthesis tools. The classifier may output a binary or probabilistic score reflecting the likelihood that a viable synthetic pathway exists for the input molecule under current algorithmic retrosynthetic constraints.

The RAscore can be utilized as a filtering metric during compound library enumeration, de novo molecular design, or virtual screening by prioritizing molecules with high predicted accessibility. This enables users to focus synthetic efforts and computational resources on compounds that are more likely to be practically synthesizable. Furthermore, the RAscore may provide guidance during generative chemistry or lead optimization workflows by rapidly identifying structures with favorable accessibility profiles according to established retrosynthetic criteria. Integration of the RAscore within broader computational pipelines may support more efficient design-make-test-analyze cycles in medicinal chemistry and related disciplines.

### Retrosynthesis-Related Synthetic Accessibility

In some embodiments, the computing system can include a synthetic accessibility (SA) model called the retrosynthesis-related synthetic accessibility (ReRSA) model, which is configured and trained to get valuable data training from all key SA modeling approaches that were discussed above in detail: retrosynthetic approach, descriptors-based approach, and data-driven approach.

In some embodiments, The ReRSA SA model operates based on the assumption that the higher the occurrence (frequency) of “synthon-like fragments” in the representative chemical space, the higher synthetic accessibility of the molecule which includes of these fragments, and preferably only these fragments. This constitutes the statistics-based core of the ReRSA method. The ReRSA SA model is configured to define what stands for “synthon-like fragment”. In the method, “synthon-like fragment” is a fragment that is automatically obtained by some predefined retrosynthesis-like decomposition procedure of molecules from a prepared training dataset that consists of real already synthesized chemical space items (e.g., input dataset). This determines to the retrosynthetic meaning of the ReRSA method. Finally, the “synthon-like fragments” and a whole assessed molecular structure itself are characterized from the perspective of structural descriptors, such as those accounting for branching, spiro-points, chiral centers and others, which are believed to influence the molecular complexity and thus indirectly synthetic accessibility. This constitutes part of the ReRSA method which is based on molecular descriptors.

In some embodiments, the protocol for obtaining “synthon-like fragments” from molecules ReRSA uses a decomposition procedure which splits a target molecule into a set (sets) of fragments. Such a decomposition function should meet several key criteria. The first one is that it has to be bijective mapping, such that it should be possible to compose a molecule back given its obtained fragments. The second criterion is that any of the resulting fragments has to be individual, such as each final fragment cannot be cleaved anymore by the reactions. The latter also means that a “synthon-like fragments” is a valid molecular structure. An example of the decomposition function that meets all mentioned criteria is an open-sourced algorithm called BRICS.

In some embodiments, the retrosynthesis-related synthetic accessibility (ReRSA) model can be employed to estimate the synthetic accessibility of candidate molecules by integrating data from multiple synthetic accessibility modeling approaches, including retrosynthetic pathway analysis, statistical fragment frequency methods, and molecular descriptor-based assessment. The ReRSA model may utilize a multi-step protocol in which a molecular structure is first subjected to a retrosynthetic decomposition to generate “synthon-like fragments” that represent plausible synthetic intermediates. These fragments may be mapped via cheminformatics algorithms, such as BRICS-based or robust retrosynthetic reaction rules, so that all resulting fragments can be recombined to reconstruct the original target molecule.

Once the decomposition is complete, the ReRSA system may quantify the frequency of each synthon-like fragment within a curated reference dataset of previously synthesized compounds. A higher occurrence of a fragment in the reference dataset is considered to correlate with greater ease of synthesis for molecules containing that fragment. The method may additionally transform synthon-like fragments into commercially available building blocks by applying conversion rules and querying vendor catalogs, such that the presence of commercially available equivalents provides a positive contribution to a molecule's synthetic accessibility score.

Structural descriptors, including but not limited to counts of chiral centers, spiro atoms, ring complexity, and molecular weight, may be computed for each fragment and for the overall molecule. These descriptors provide additional assessment of structural complexity and can alter the synthetic accessibility scoring accordingly. The model can further reward molecules that have multiple distinct retrosynthetic “splits” or possible decomposition pathways, particularly when those splits are structurally diverse and result from different retrosynthetic strategies.

The ReRSA model may also analyze the types of virtual reactions used in the decomposition; for example, multi-component reactions may increase the accessibility score, while macrocyclization steps or the presence of rare or synthetically irrelevant substructures can incur scoring penalties. Policy selection interfaces (STRICT or SOFT) can allow a user to prioritize either rigorous filtration of novel or underrepresented fragments or a more permissive scoring approach that penalizes but does not exclude such structures.

To provide the result, the ReRSA scoring process aggregates frequencies of synthon-like fragments, building block availability, structural descriptor scores, split diversity, reaction type contributions, and substructure penalties to output a unified synthetic accessibility score for the input molecule, normalized to a user-friendly scale. The ReRSA workflow can be integrated into automated molecule selection, generative chemistry design, or synthetic route planning platforms, providing batch evaluation and visualization features, such as annotated structure diagrams with available building blocks, CAS registry data, and vendor links, to facilitate informed decision-making in drug discovery, library enumeration, and retrosynthesis planning.

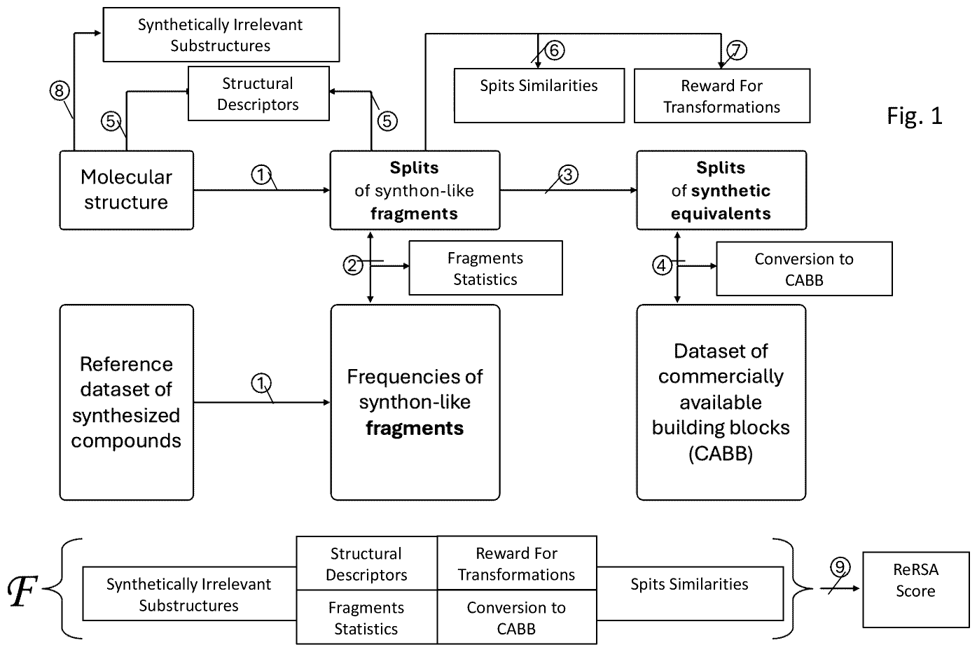

In this way the principal schema with the key steps of the new ReRSA scoring algorithm are described in FIG. 1. FIG. 1 includes a schematic representation of the ReRSA scoring algorithm. In some embodiments, a description of the principal schema of the ReRSA scoring algorithm is provided.

### Fragments Statistics (Frequencies)—Procedure 1

The molecular structure scoring procedure is preceded by the reference dataset preparation and pre-calculation (model training) of synthon-like fragments statistics in the reference dataset. The process of pre-calculation starts from the quasi-retrosynthetic decomposition of molecular structures from the reference dataset into synthon-like fragments (e.g., procedure 1 is reference dataset of synthesized compounds is processed for frequencies of synthon-like fragments). The synthon-like fragments are organized in so-called splits (e.g., sets of fragments) which are then aggregated into the homogenous dataset of fragments in order to calculate the frequencies of fragments in the reported chemical space. The result of procedure 2 is the dataset of fragments with their aggregated frequencies. Higher frequencies have a higher contribution to the synthetic accessibility of the molecular structure. Procedure 2 is the performance of fragments statistics for frequency of fragment of synthon-like fragments to obtain the dataset of fragments with fragment statistics.

The process of target molecular structure assessment for synthetic accessibility starts from its quasi-retrosynthetic decomposition into sets of synthon-like fragments (splits) (procedure 1 on FIG. 1). In these terms the procedure of target molecule decomposition is equal to the procedure 1 of reference dataset decomposition into synthon-like fragments. As soon as the splits of synthon-like fragments are obtained they are converted into the splits of synthetic equivalents (procedure 3). The procedure 3 is the splits of synthon-like fragments is processed to obtain splits of synthetic equivalents, whereby the synthetic equivalents are determined.

### Conversion to Building Blocks (CABB)—Procedure 4

The synthetic equivalents are then indexed in a prepared dataset of commercially available starting materials (procedure 4). The more synthetic equivalents are found in the dataset the higher contribution to the final estimate of synthetic accessibility is obtained for the structure. Procedure 4 is the determination of commercially available building blocks (CABB) or conversion of synthon-like fragments to commercially available building block (CABB) fragments.

### Structural Descriptors Values—Procedure 5

In order to take into account the context of the molecular complexity of synthon-like fragments and the target molecular structure are described within structural descriptors (procedure 5). The descriptor values are stored for each fragment. The procedure 5 uses structural descriptors with descriptor values for use to take into account the context of complexity of the synthon-like fragments and molecular structure.

### Diversity/Similarities of Splits—Procedure 6

In order to reward molecular structures possessing multiple diverse options of synthesizing them, the splits are analyzed for their similarities (procedure 6). The more diverse splits are, the higher contribution to the final estimate of synthetic accessibility is obtained for the structure. The procedure 6 includes analyzing the splits of synthon-like fragments for similarities in structures. Similar structures may not be rewarded highly, but multiple diverse options of molecular structures for the splits of synthon-like fragments can be highly rewarded.

### Reward for Reaction Type—Procedure 7

Additionally, the virtual reactions applied to the molecular structures are analyzed for their influence on the synthetic accessibility. Multi-component reactions are rewarded, while macrocyclization reactions are penalized (procedure 7). Procedure 7 includes analyzing molecular structures of the synthon-like fragments for synthetic accessibility, which includes multi-component reactions being rewarded and any macrocyclization being penalized.

### Synthetically Irrelevant Substructures Penalization—Procedure 8

Alongside the big library of pre-computed synthetically irrelevant SMARTS-encoded substructures is applied to the molecular structure for an additional penalty (procedure 8). Procedure 8 can then analyze the synthon-like fragments or substructures thereof for pre-determined substructures that are irrelevant and not useful, which can be given lower scores or omitted.

### ReRSA Score is Calculated—Procedure 9

Finally, the overall ReRSA score is calculated (procedure 9). Procedure 9 is the calculation of the ReRSA score according to the function (F) aggregating 6 factors and corresponding procedures results: (1) Fragments statistics (frequencies)—Procedure 1; (2) Conversion to building blocks (CABB)—Procedure 4; (3) Structural descriptors values—Procedure 5; (4) Diversity/similarities of splits—Procedure 6; (5) Reward for reaction type—Procedure 7; and (6) Synthetically irrelevant substructures penalization—Procedure 8.

### Training Procedure

The key goal of the training procedure is to get statistics for synthon-like fragments from the real synthetical chemical space.

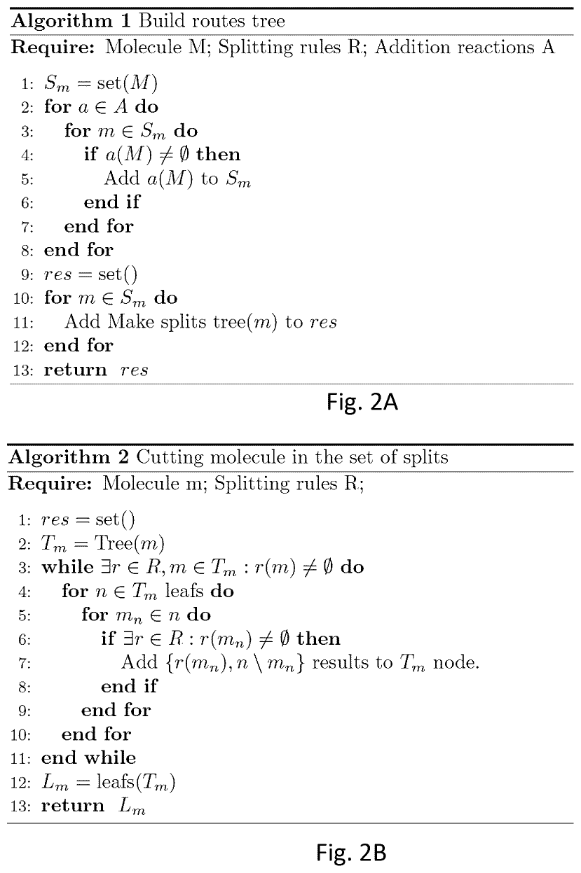

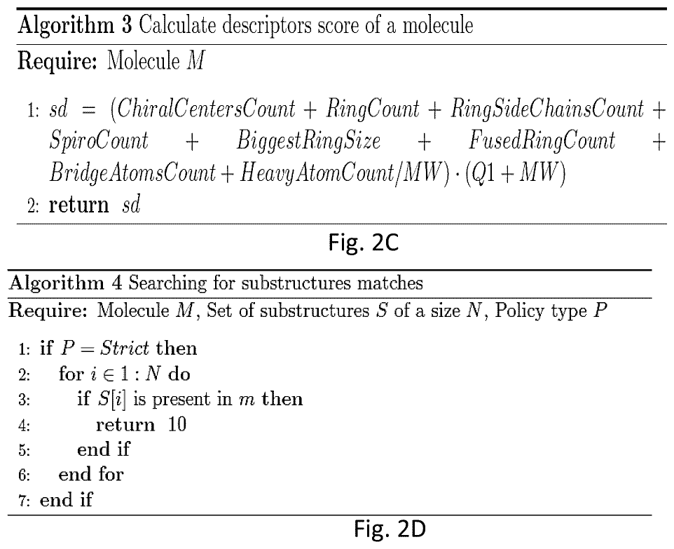

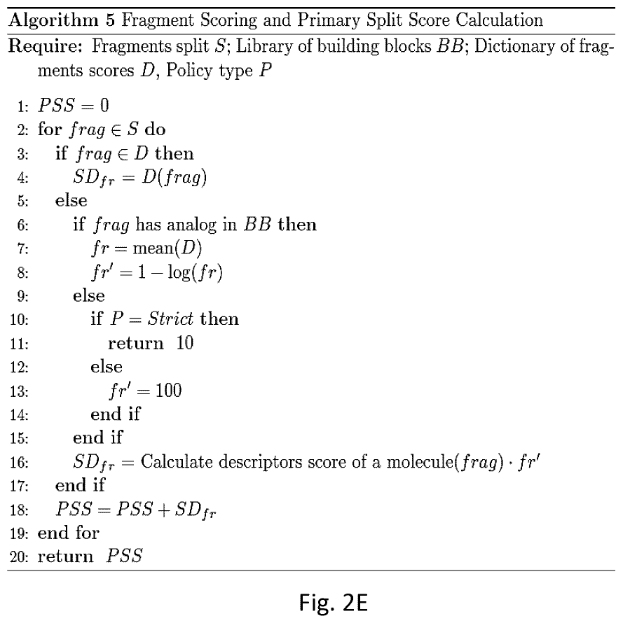

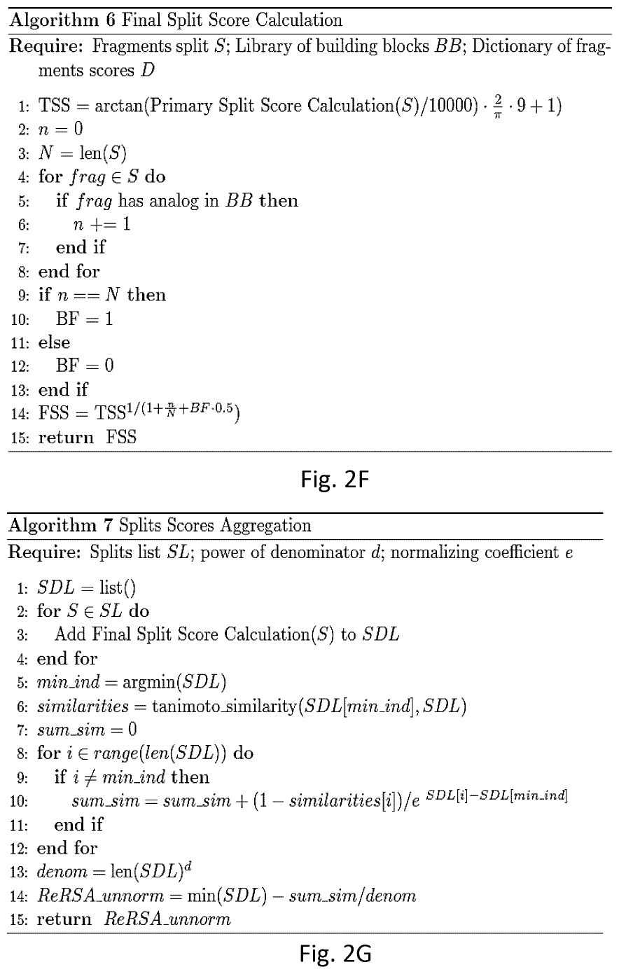

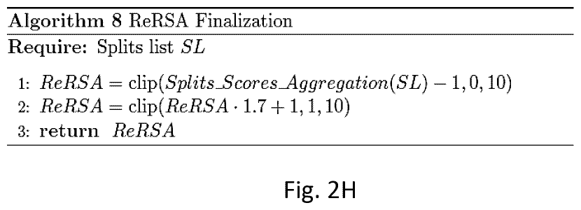

Step 1 of the training procedure can include the decomposition as described herein. During the training each molecule from the training dataset is decomposed into different splits of fragments and building blocks using robust reactions. The procedure resembles the process of retrosynthesis tree building, where each independent leaf of the tree stands for a split (see Algorithm 1 in FIG. 2A and Algorithm 2 in FIG. 2B).

Step 2 of the training procedure can include building a fragments dictionary database. All unique fragments are collected in a dictionary and their frequencies are calculated based on their presence in reference synthetic space (for example, ChEMBL dataset). Frequency of a fragment (fr) is the number of molecules from a prepared training dataset containing the fragments, divided by the total number of molecules in the training dataset so it will always be between zero and one. Therefore, if the frequency of a fragment is low it will contribute much to the higher ReRSA score of the method and vice versa. The higher ReRSA values mean the lower synthetic accessibility and vice versa). In other words, rare synthesized fragments are harder to synthesize than frequent ones. While frequencies can be used as is, the approach takes a minus logarithm of it (fr′):

$${fr}^{′} = 1 - log ( fr ) ,$$

for which fr′ makes a bigger contribution to the overall score (e.g., bigger fr′ values stand for lower synthetic accessibility).

Every unique fragment also gets its structural descriptors values score component (sd) (see Algorithm 3 in FIG. 2C):

The final subscore for each fragment (SD) is calculated as the result of the function taking into account descriptors and fragment frequency in a dictionary over the whole training dataset (see Algorithm 3 in FIG. 2C):

$$SD = Calculate descriptors score of a molecule ( M ) \cdot {fr}^{′} .$$

In some embodiments, SD score represents chemical complexity of the fragment in terms of its usage in the training dataset and its structural properties described within carefully selected well-tuned molecular descriptors (MDs) which are defined as follows: ChiralCentersCount—the number of chiral carbon atom, RingCount—the total number of rings, RingSideChainsCount—the number of side chains attached to the ring systems, SpiroCount—the number of spiro carbon atoms, BiggestRingSize—the number of atoms in the largest ring of molecular structure if it is bigger than 6, otherwise 0, FusedRingsCount—the number of fused rings in a molecular structure, BridgeAtomsCount—the number of bridgehead atoms in the bicyclic pattern(s) of molecular structure, HeavyAtomCount—the number of atoms other than hydrogen, MW—molecular weight value, Q1—normalized quadratic index 1 calculated as (3−2·A+Z1/2), where A is the number of heavy atoms, and Z1 is the first Zagreb index.

All MDs in the formulas of SD Score have a strong chemical relevance and highly correlate with the complexity of the fragment meaning that from a chemical point of view the increase in any MD of the fragment should definitely increase its entanglement and complexity.

### Scoring Procedure

The key goal of the scoring procedure is to get a ReRSA score for a random molecule.

In a preliminary step (e.g., Step 0), a primary structure normalization is performed and a check for abnormalities is conducted. First, the ReRSA algorithm gets as an input the SMILES-string of a molecular structure or a real molecule, such as from a database of real molecules. Then the default RDKit normalization procedure (canonicalization) is applied where the valence, the charge and the aromaticity of the atoms are checked. Only if the molecular structure passes this procedure, its canonicalized SMILES-string is fed into the ReRSA engine. Otherwise, an error is displayed or the molecule is omitted. Thus, only chemically and structurally valid inputs are applicable for ReRSA.

The scoring procedure starts within the Algorithm 4 in FIG. 2D, which allows to find synthetically irrelevant substructures in a molecular structure from a predefined library using SMARTS queries including but not limited to any optionally substituted cycles including single cycles, fused, spirocycles; any optionally substituted linkers and ring decorations. Algorithm 4 applies only when the STRICT policy is chosen. In this case, if the irrelevant substructure was found, ReRSA=10 is returned and further operations (Algorithms 5 (FIG. 2E), 6 (FIG. 2F), 7 (FIG. 2G), and 8 (FIG. 2H)) are not performed. When the SOFT policy is applied, Algorithm 4 is skipped and Algorithms 5-8 go as usual.

In a first step (e.g., Step 1) a decomposition of the molecule is performed. The model can receive a new molecule and decompose it into a set of splits according to Algorithms 1 and 2.

In a second step (e.g., Step 2) fragments scoring and primary split score calculations are performed. For all fragments (frag) in each split (S) we get SD values checking for availability of the fragments (frag) in the fragments dictionary (D) generated at the training step. If a fragment is available in the dictionary, its SD value is taken automatically. If not, the SD value is calculated according to modified Algorithm 5, where the fragment frequency is taken based on convertibility of the fragment into a commercially available building block (CABB) from a building block library (BB). If a CABB is found in BB the fragment gets the mean frequency (mean fr) over the whole training dataset, if not, the fr′ value of the fragment will be 100. All taken or calculated SD values then summarized into the Primary Split Score (PSS) in Algorithm 5 in FIG. 2E.

In a third step (e.g., Step 3), normalization and final split score calculation is performed. It can be seen from the Step 2 above that the Primary Split Score (PSS) can take values from zero to infinity, so it is not normalized. To make the split scores more user-friendly and meaningful in terms of medicinal chemistry a plenty of normalizing functions can be employed. For instance, if the desired value of the score should be between zero and one then a sigmoid function can be used. To achieve the score in a specific predefined range one can for example apply an arctangent function with some range specific parameters. In the case of arctangent, the Primary Split Score undergoes the following mathematical transformation:

$$T SS = arctan ( \frac{PSS}{10000} ) \cdot \frac{2}{π} \cdot 9 + 1.$$

After this transformation we get the Transformed Split Score (TSS). The Final Split Score (FSS) is calculated from the Transformed Split Score taking into account the number of fragments of the split that have been successfully converted into CABB. The more fragments are converted into CABB, the lower is Final Split Score (see Algorithm 6 in FIG. 2F).

In a fourth step (e.g., Step 4) the split scores are aggregated into the ReRSA score. Then, all Final Split Scores are aggregated into the ReRSA score of a molecule taking into account the differences between the best Split (the Split with the lowest Final Split Score) and other splits in 1) split-to-split similarity (Tanimoto score)—dissimilar splits contribute more and 2) actual Final Split Scores—splits with lower Final Split Score contribute more (see Algorithm 7 in FIG. 2G).

In a fifth step (e.g., Step 5) the finalization of the ReRSA score includes smoothing and normalization. Smoothing is used to avoid sharp drops in ReRSA scores over the chemical space and clipping is applied to fit ReRSA scale from 1 to 10 (see Algorithm 8 in FIG. 2H). This is the final step of calculations, here we get the final ReRSA score for a random molecule.

### Examples

In order to describe the capabilities of the updated ReRSA algorithm (ReRSA 3.0) and to compare it with other synthetic accessibility estimators we have carried out a few experiments that are described below.

### Experiment 1—Splits

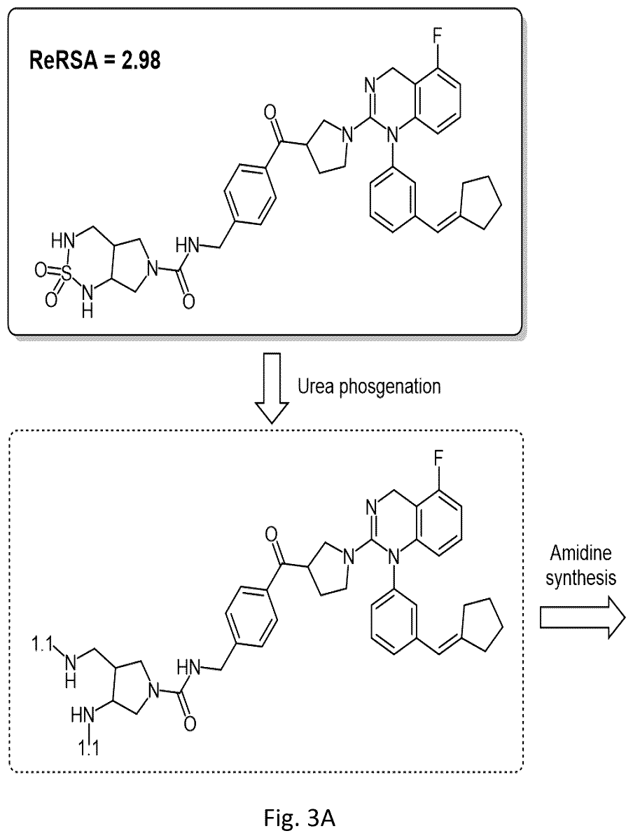

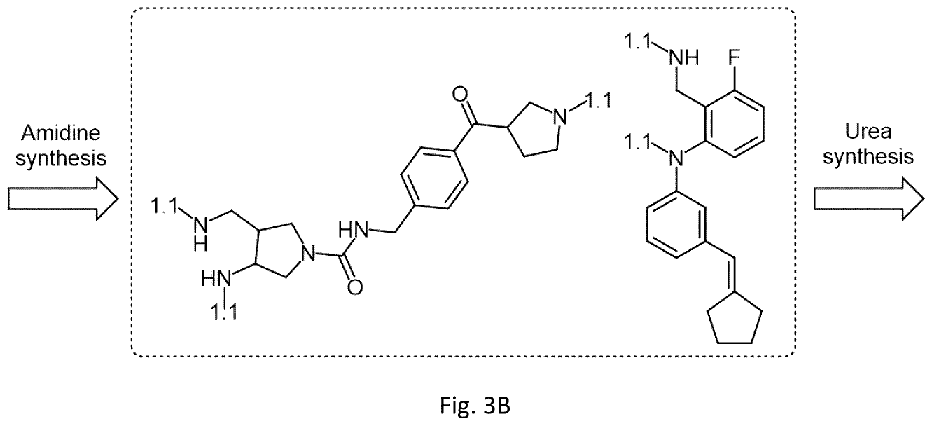

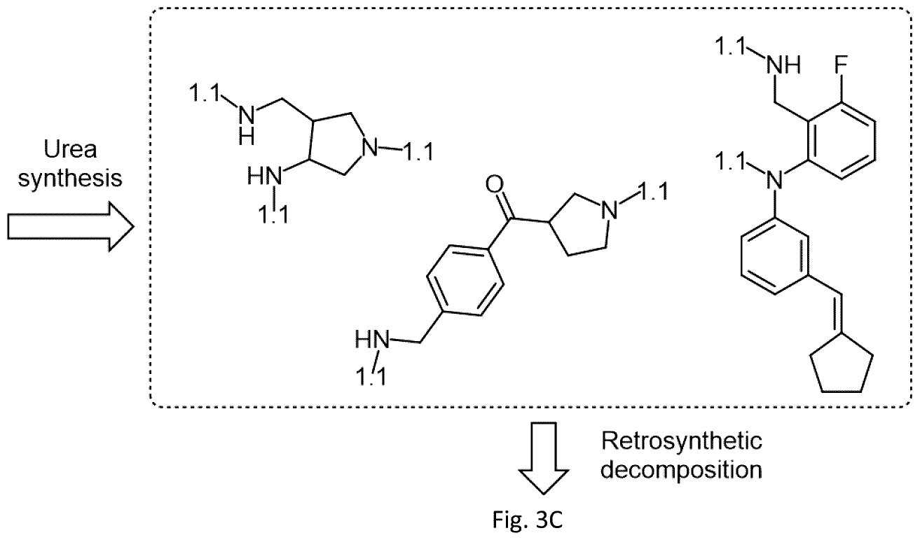

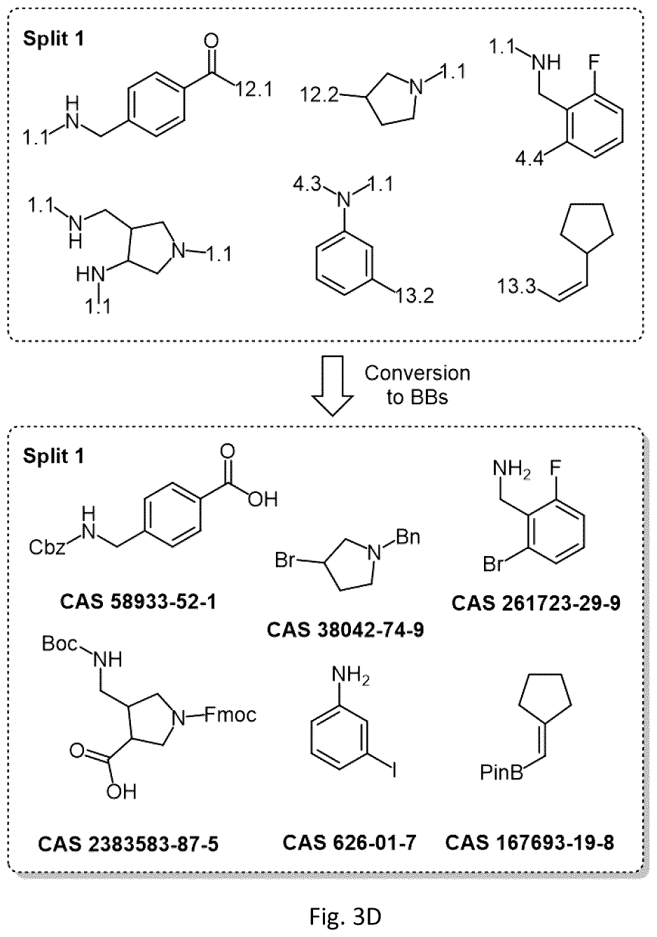

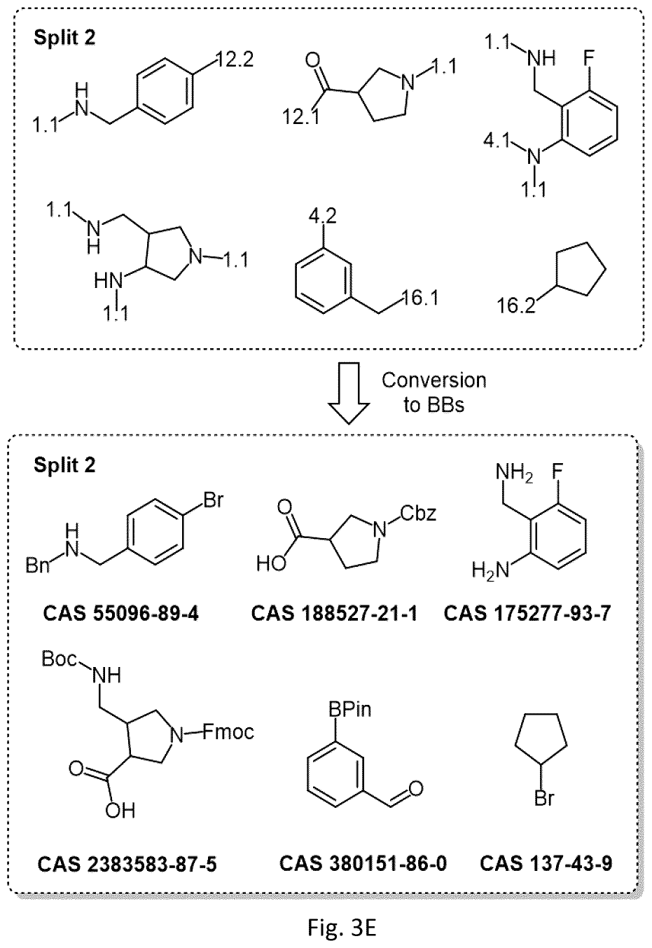

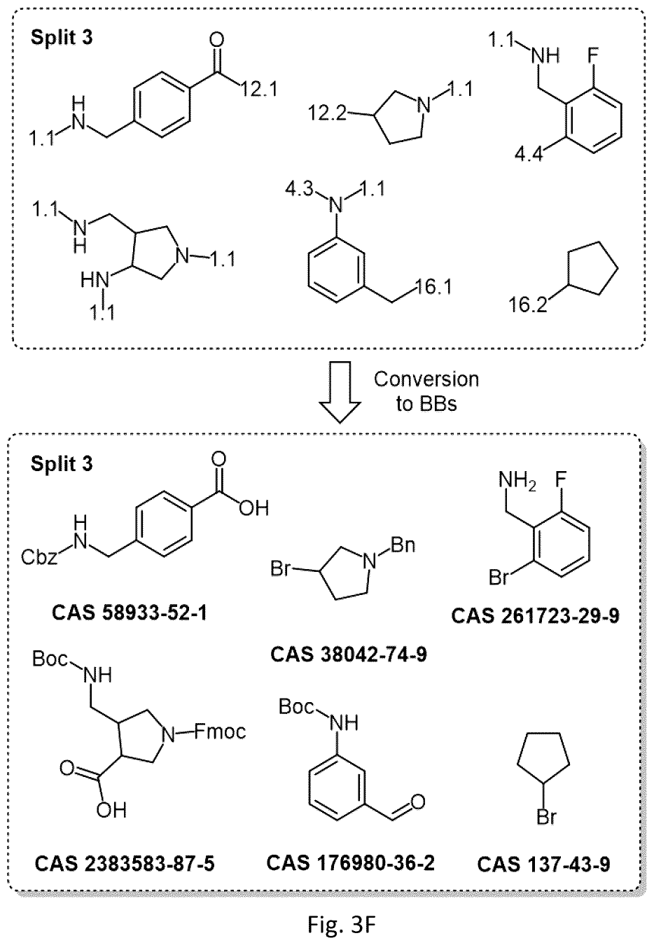

The ReRSA provides implementation of quasi-retrosynthetic logic leading to the formation of so-called splits, which are the outcomes of mapped potential building blocks derived from the separate branches of quasi-retrosynthetic tree. The examples of splits and their formation are given in FIGS. 3A-3F, FIGS. 4A-4C, and FIGS. 5A-5C.

The target molecule which is an example from the generative chemistry outcome (see FIGS. 3A-3F) is preliminary simplified using the heterocycle retrosynthesis (diamine phosgenation and cyclic amidine synthesis), then using urea synthesis. After these three retrosynthetic reactions applied the target molecule is split into three smaller ones (intermediates) and there are at least three more sets of BRICS-like reactions (retrosynthetic decomposition) that can be applied to the retrosynthetic intermediates to get at least final three splits of fragments that cannot be split anymore further. The final fragments are then converted into building blocks using conversion rules and the resulting building blocks are finally compared to the dataset of commercially available starting materials. According to the conversion rules more than one option can be applied for each fragment type. For example, fragment type No 4.4 can be converted into chlorine, bromine and iodine atoms. Finally, all available options will be provided, if the converted building blocks are found in the reference starting materials dataset.

FIGS. 3A-3F illustrate decomposition of the dummy molecular structure into splits.

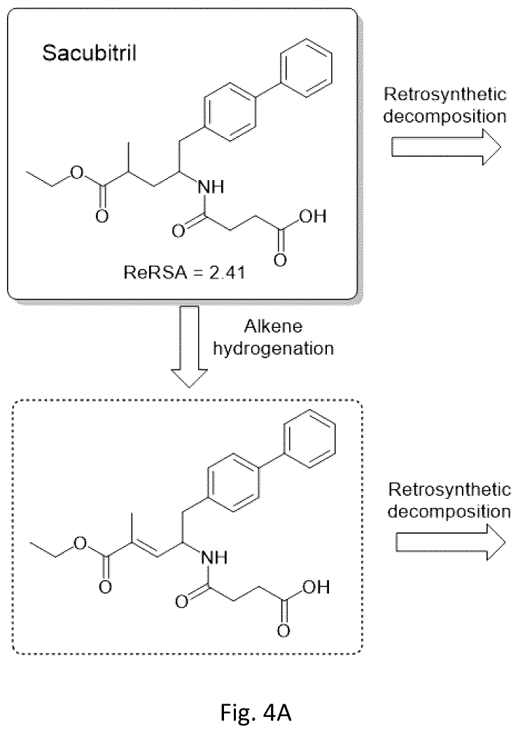

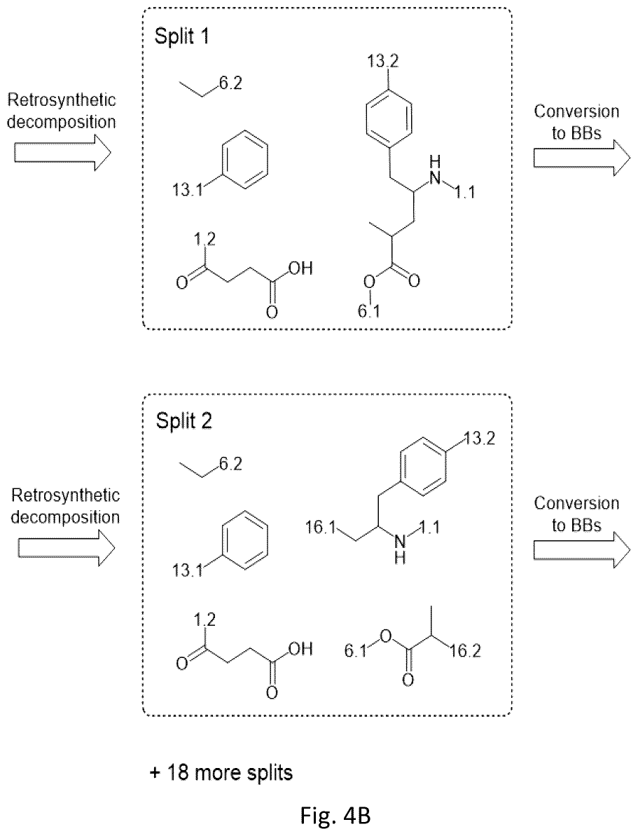

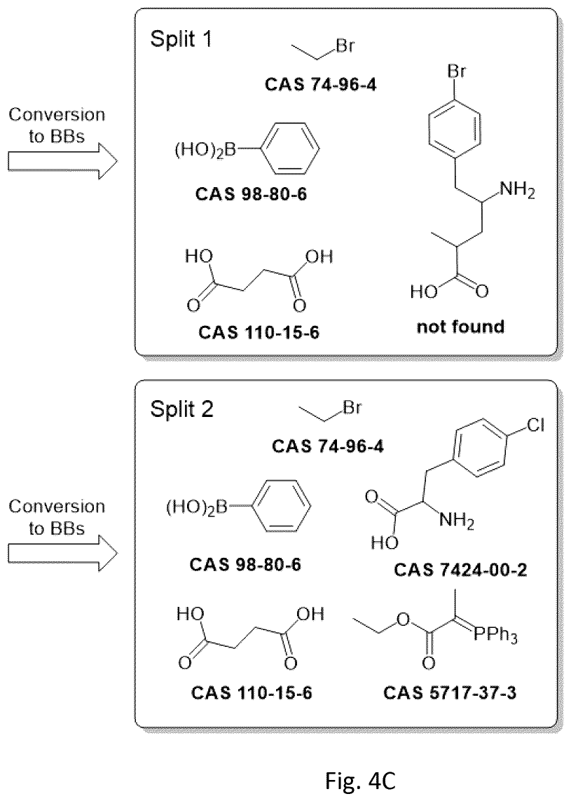

Sacubitril (FIG. 4A)—an FDA approved antihypertensive drug, can be decomposed in at least two ways. The first one is that ultimate BRICS-like retrosynthetic decomposition can be applied to sacubitril directly (Split 1), while the second one should involve a preliminary step of alkene hydrogenation allowing for the application of Wittig reaction at the further steps (Split 2). Here we can see that application of preliminary reactions can be beneficial for deeper retrosynthetic conversion in comparison to application of BRICS-like reactions only. FIGS. 4A-4C show the decomposition of sacubitril where only the splits with the highest scores are provided.

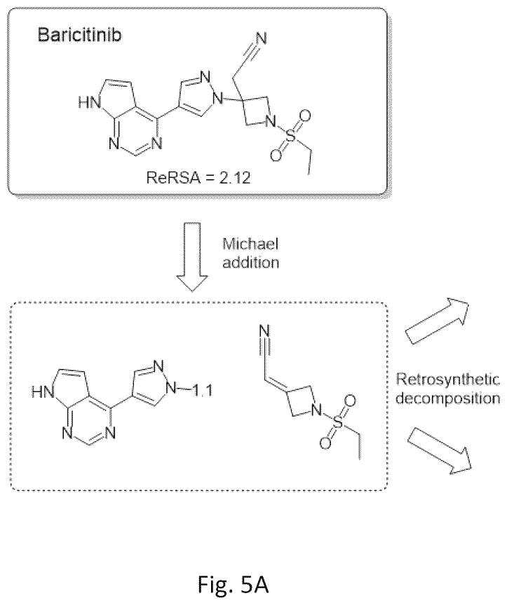

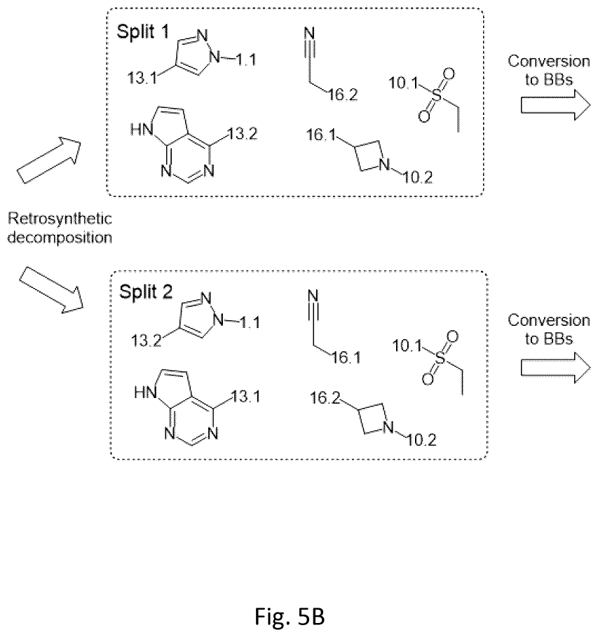

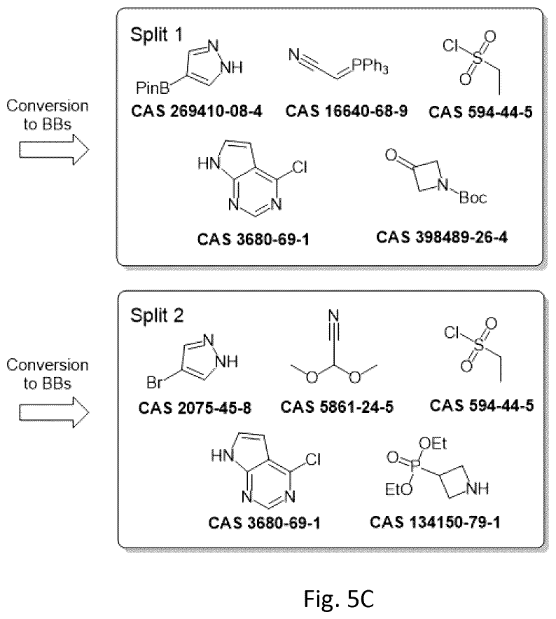

Each BRICS-like retrosynthetic-decomposition reaction can be applied bilaterally. This can be illustrated by the application of the ReRSA 3.0 algorithm to Baricitinib (see FIGS. 5A-5C). First, the preliminary retrosynthetic reaction of Michael addition-like conjugate addition of NH-heterocycle to α,β-unsaturated nitrile is applied. Then the BRICS-like retrosynthetic-decomposition reactions are applied to intermediates and the Wittig/HWE-decomposition (fragment types 16.1 and 16.2) is applied bilaterally to get different splits (Split 1 and Split 2). Finally, both phosphorus ylide, the retro-product of retro-Wittig reaction (CAS 16640-68-9), and phosphonate, the retro-product of retro-HWE reaction (CAS 134150-79-1), respectively are found in the dataset of commercially available starting materials. FIGS. 5A-5C show the decomposition of baricitinib where only the splits with the highest scores are provided.

### Experiment 2—Building Block Availability Sensitivity Assessment

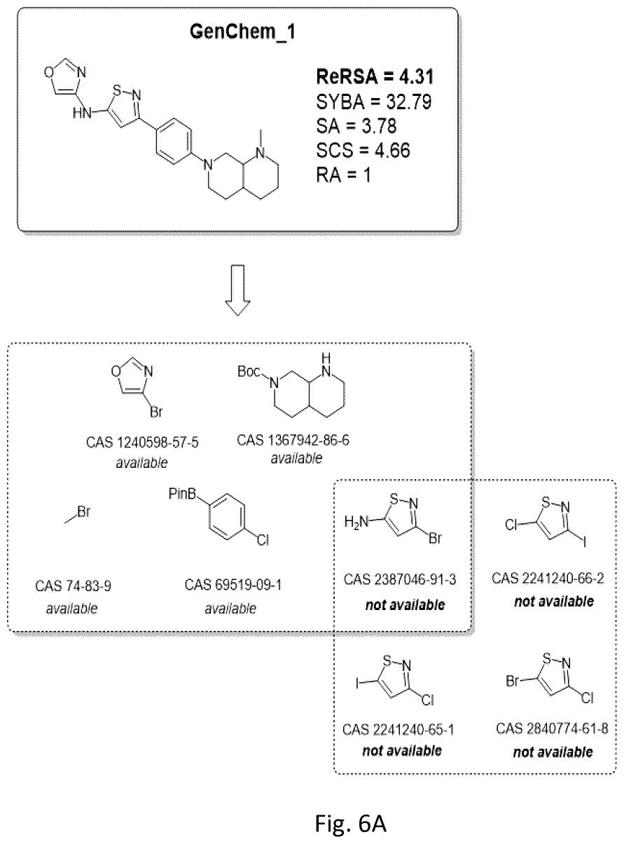

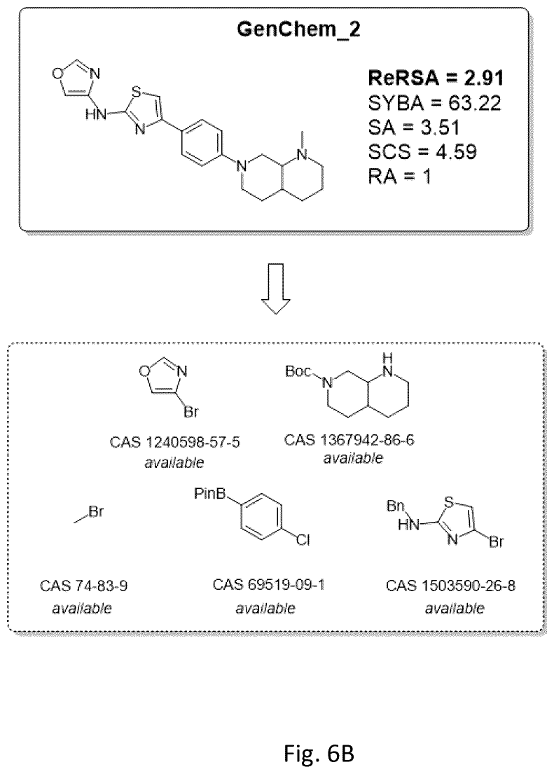

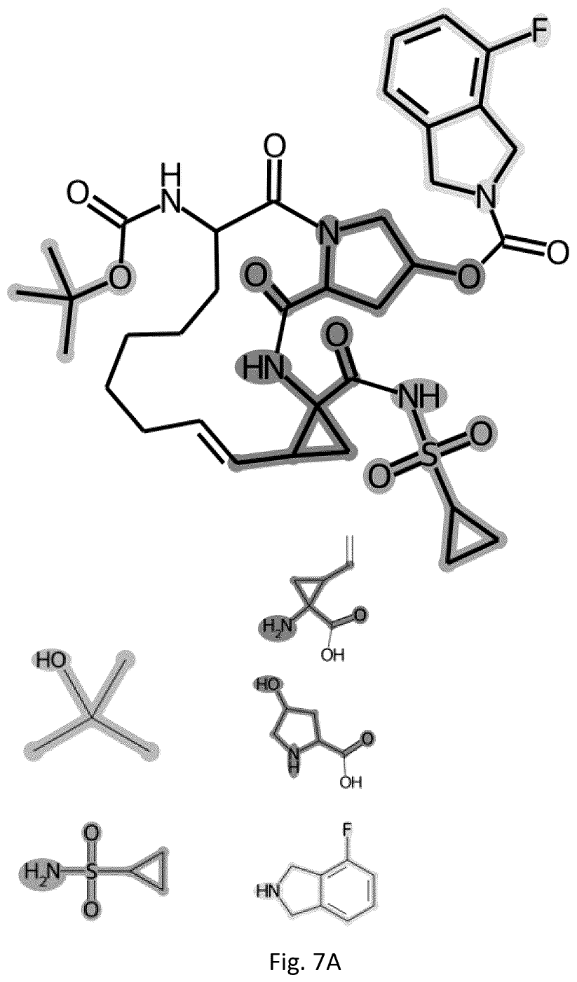

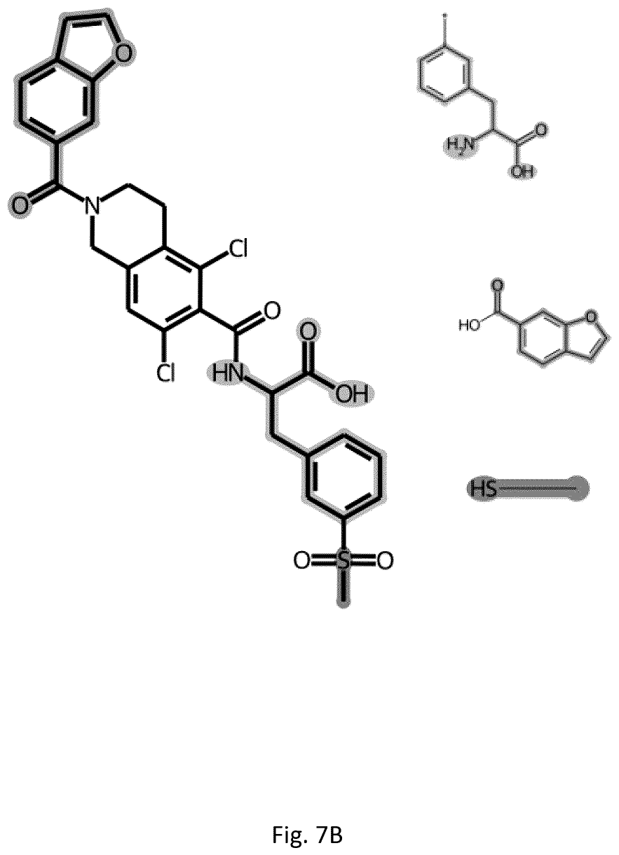

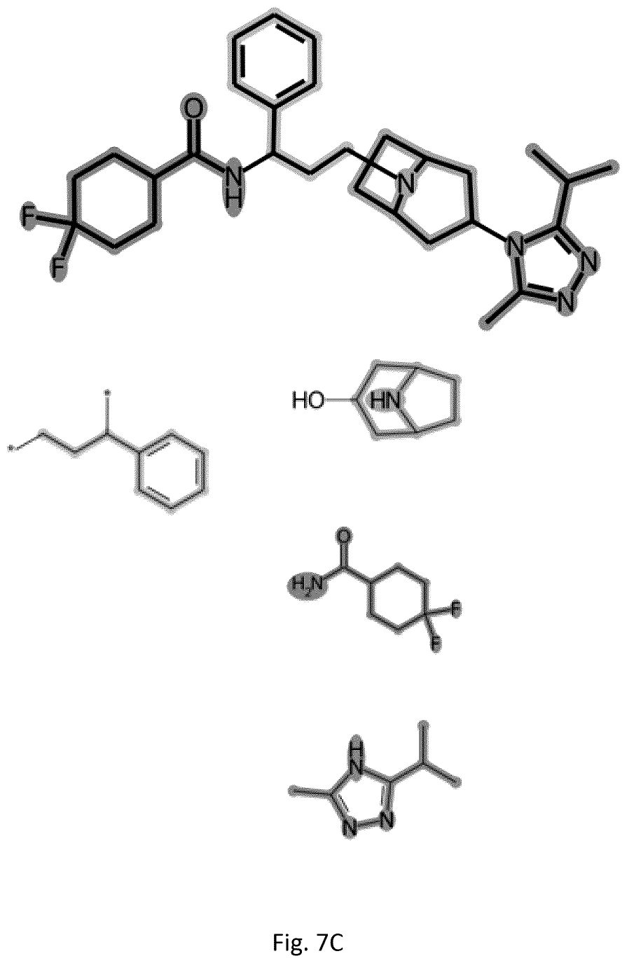

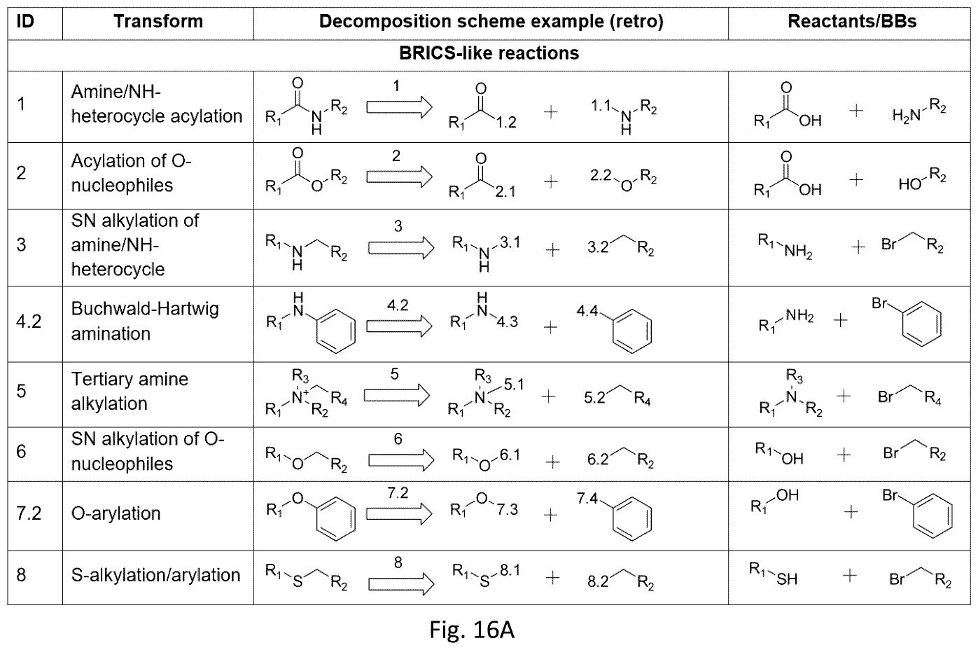

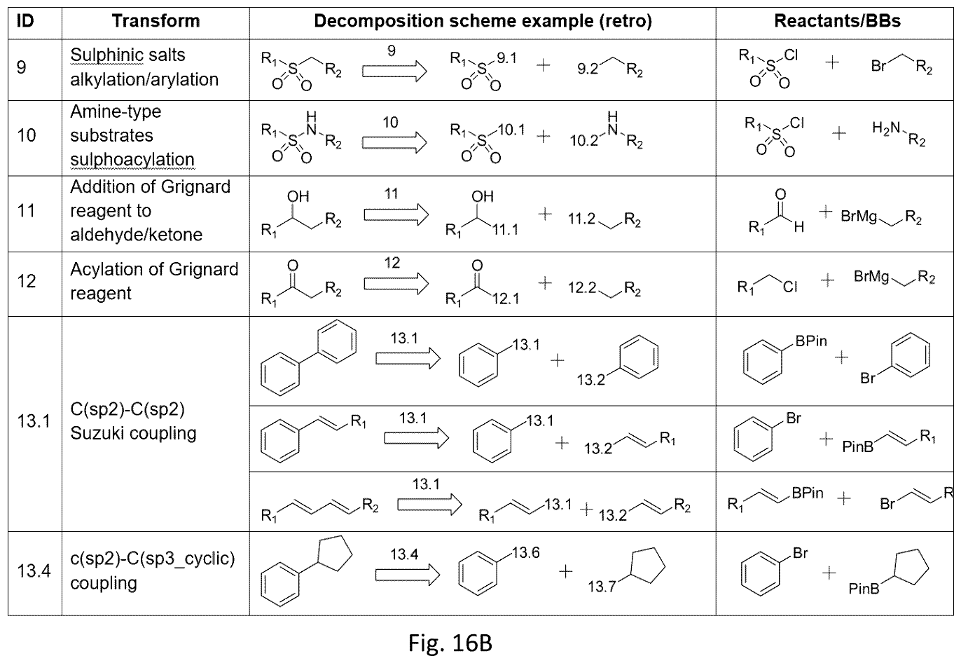

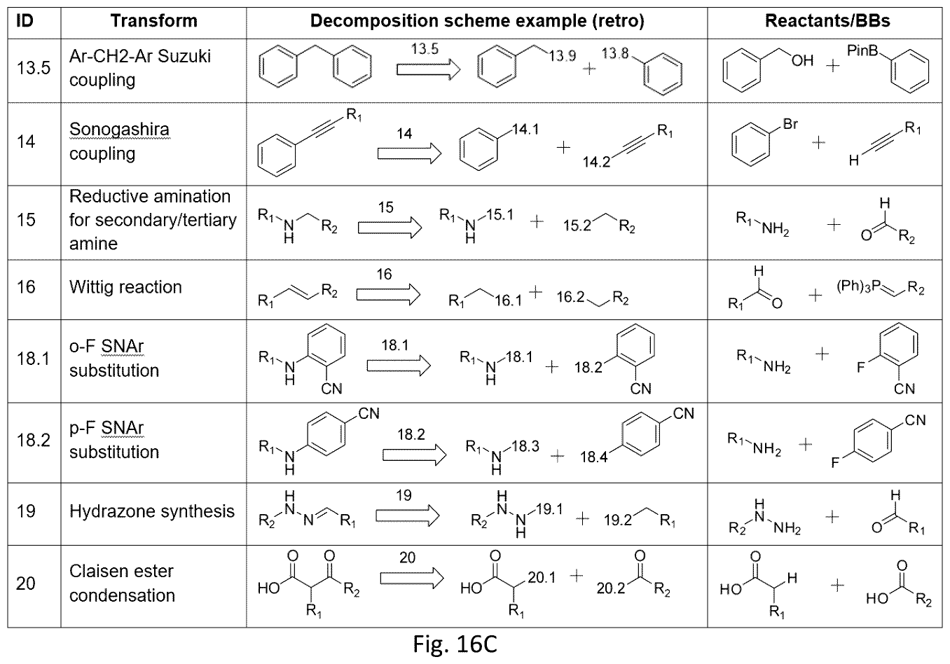

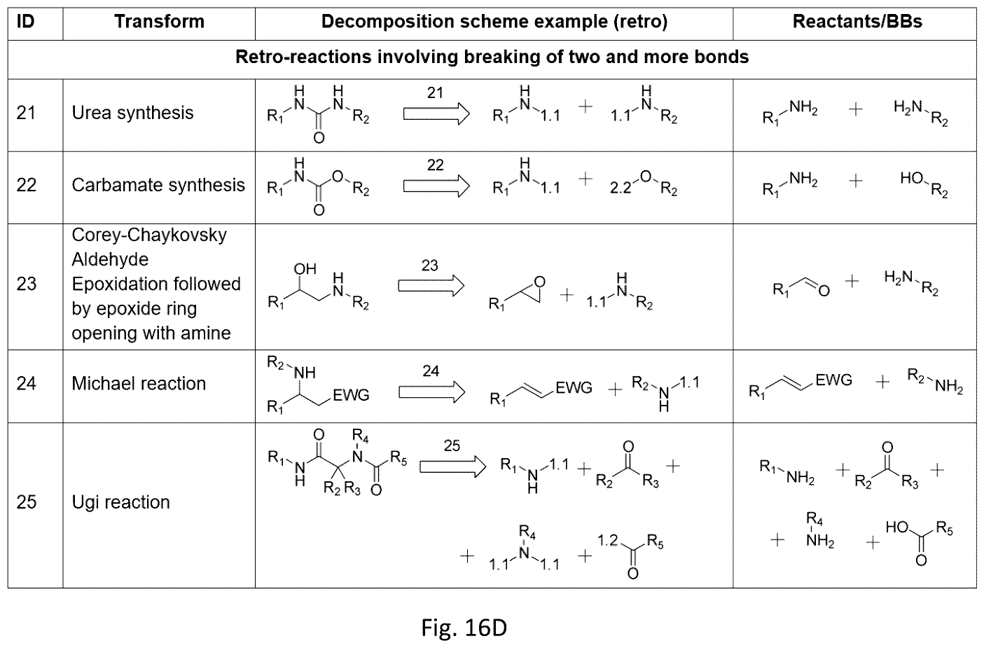

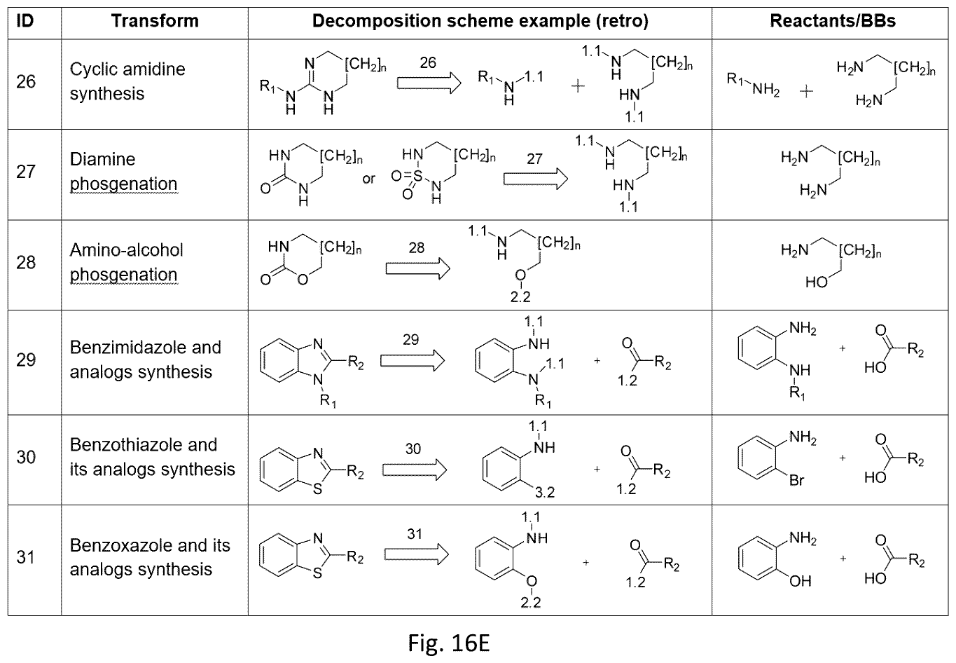

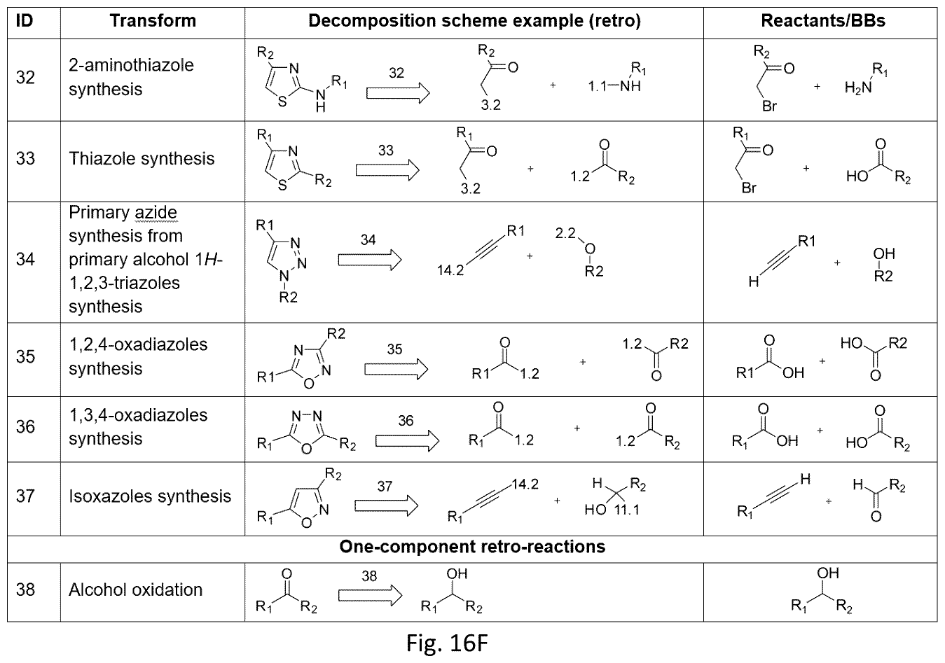

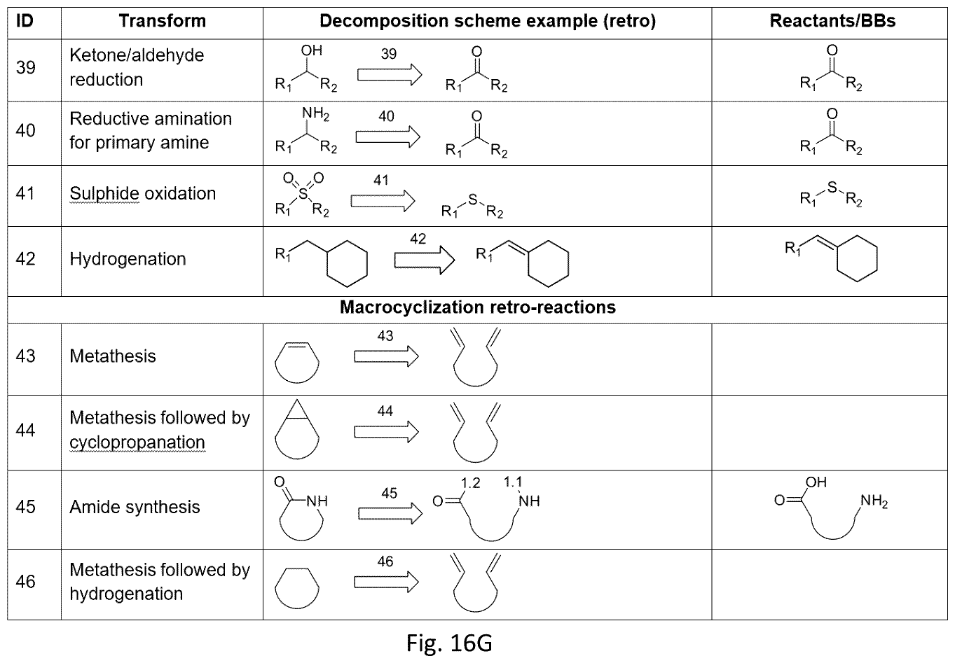

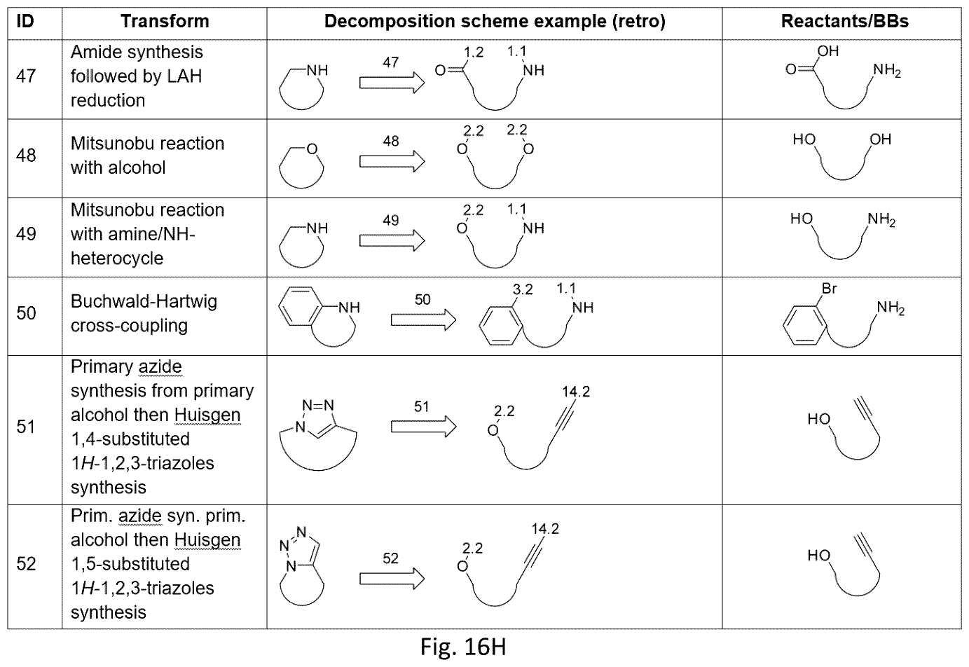

Availability of a building block that could be used to synthesize a molecule is a crucial condition to consider the molecule as synthetically accessible. The presence of a CABB in the retrosynthetic route is one of the key points that are taken into account by the ReRSA algorithm. If a molecule can be split by robust reactions (FIGS. 16A-16H) into the set of CABB, it will be rewarded in terms of low ReRSA scores. As it is exemplified below (see FIG. 6A-6B), two sample structures from the generative chemistry platform Chemistry42 have been assessed for synthetic accessibility using multiple methods including ReRSA. Both structures can be split into building blocks using robust reactions such as Suzuki coupling, Buchwald-Hartwig arylation and SN2 methylation. However, only GenChem_2 can be split into a full set of CABB, since all isothiazole BBs after proper analysis in SciFinder database can be considered as not available at vendor stock and ready to be delivered in meaningful amounts. Among all tested methods to assess synthetic accessibility only the ReRSA algorithm can take this BBs context into consideration.

FIGS. 6A-6B illustrate two samples from the generative chemistry platform Chemistry42 assessed for synthetic accessibility and distinguished within ReRSA score.

### Experiment 3—ReRSA Scoring Supported by Building Blocks Visualization

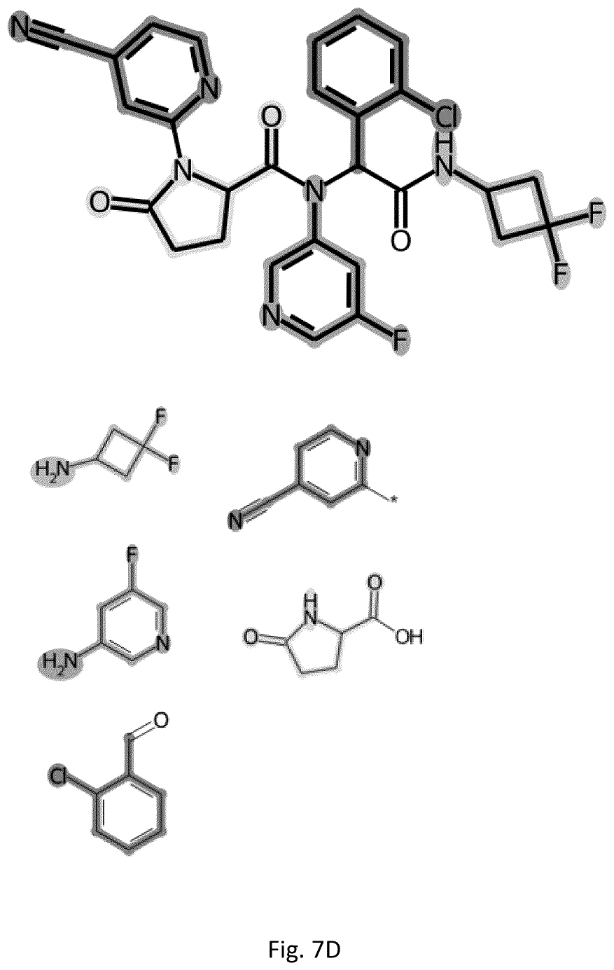

The ReRSA scoring is supported by building blocks visualization available at the Chemistry42 platform. The unchangeable parts of building blocks that are found in the target molecule are highlighted. Also CAS numbers are provided for building blocks as well as links to their webpages at the PubChem website. The homogenous building block options (e.g., aryl halides: chloride, bromide and iodide) are provided for more options to solve selectivity problems post factum. If some parts of a molecular structure are not converted into commercially available building blocks the corresponding disclaimer is provided. Examples (screenshots. FIG. 7A-7D) of ReRSA visualization are provided from the Chemistry42 platform for partially converted marketed drugs, fully converted marketed drugs and examples from generative chemistry pipeline respectively are provided.

FIG. 7A includes visualization of ReRSA output from Chemistry42 for danoprevir (partially converted drug).

FIG. 7B includes visualization of ReRSA output from Chemistry42 for lifitegrast (partially converted drug).

FIG. 7C includes visualization of ReRSA output from Chemistry42 for maraviroc (fully converted drug).

FIG. 7D includes visualization of ReRSA output from Chemistry42 for ivosidenib (fully converted drug).

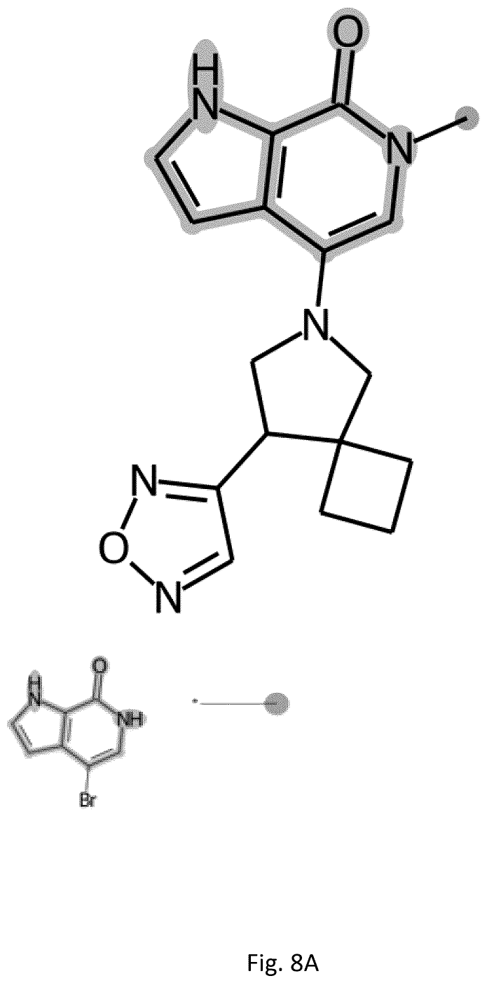

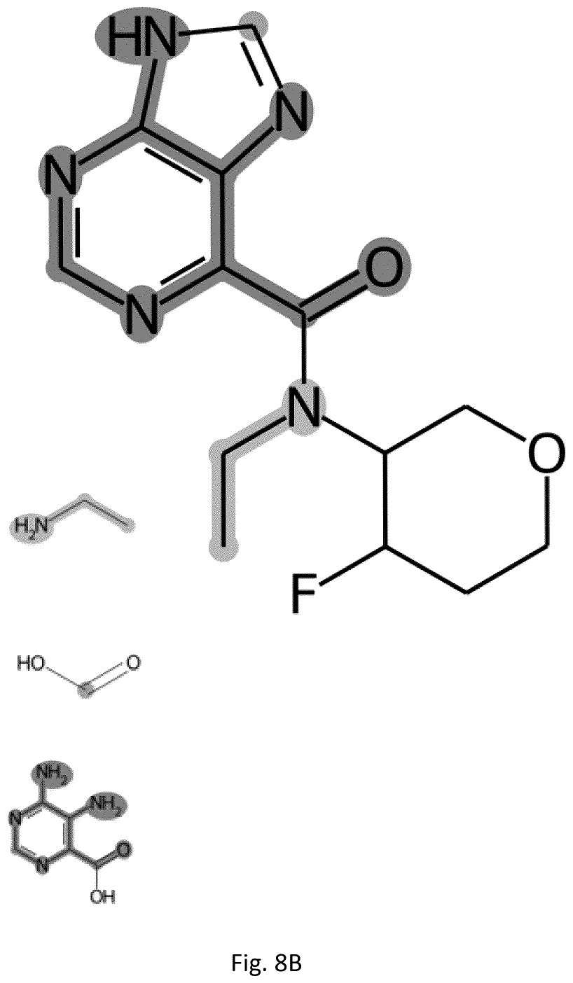

FIGS. 8A-8B include visualization of ReRSA output from Chemistry42 for 2 partially converted generated structures.

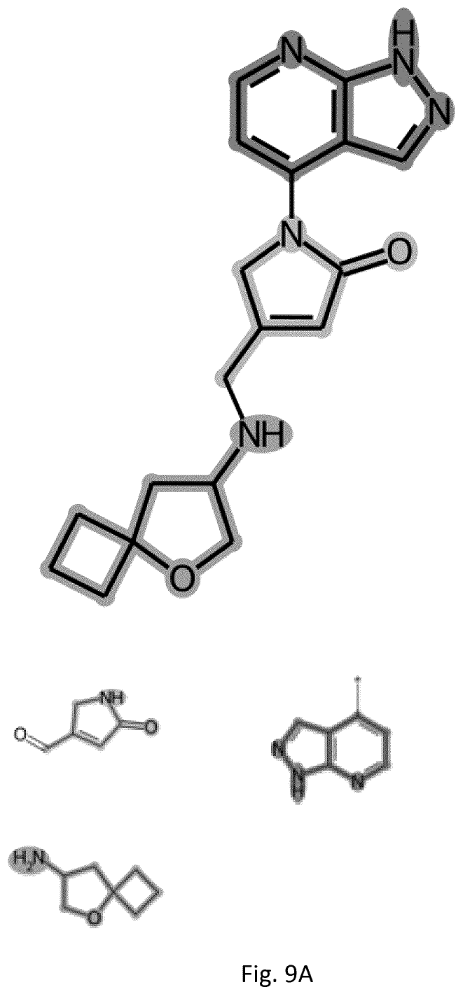

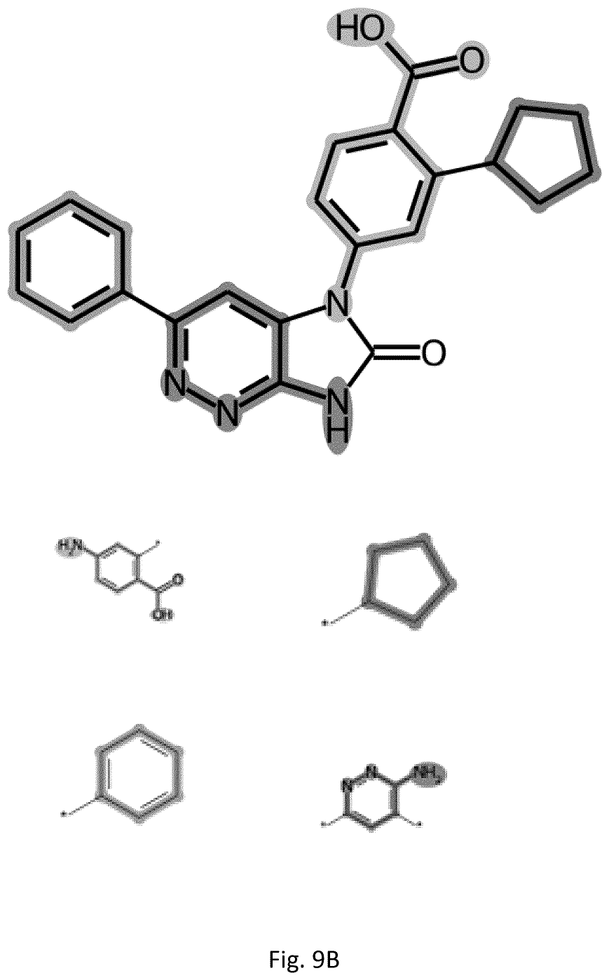

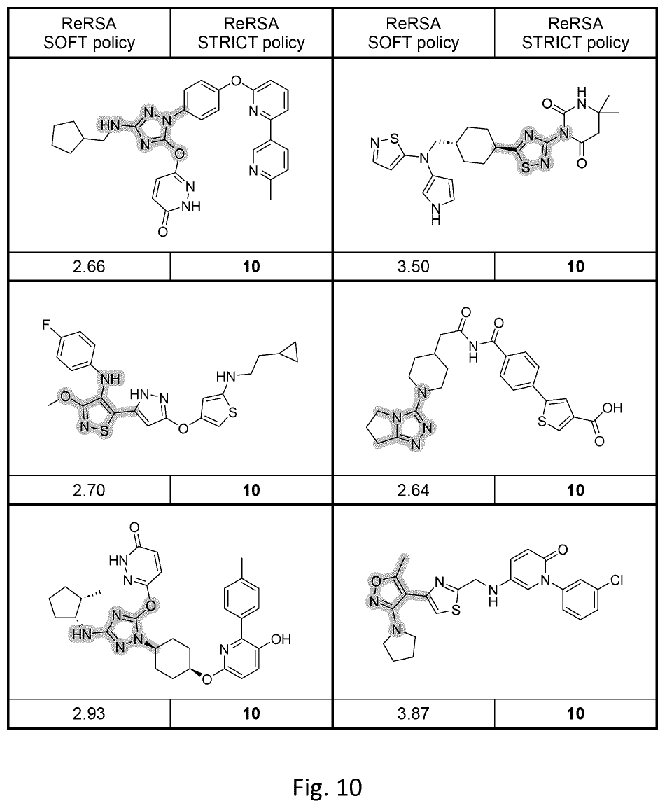

FIGS. 9A-9B include visualization of ReRSA output from Chemistry42 for 2 fully converted generated structures.

### Experiment 4—ReRSA Score as a Measure of Molecular Complexity

The concept of synthetic accessibility is strongly related to the concept of molecular complexity, which is still important for drug design and chemical space analysis but cannot be strictly unified by the members of the professional community. While there are no widely-used metrics to measure molecular complexity, ReRSA scores can be considered as one of the metrics to estimate molecular complexity. The complexity in terms of ReRSA score is based on three assumptions:

The presence of bonds that can be cleaved using robust retro-reactions (e.g., amide synthesis, Suzuki coupling etc.) means that a structure can be decomposed into synthons making it less complex. Rare fragments (synthons), that were not observed in the reference dataset, contribute to the lower synthetic accessibility that means the decomposed structure is more complex. The presence of structural features such as chiral centers, spiro cycles, fused rings and others contribute to lower synthetic accessibility making the molecular structure more complex.

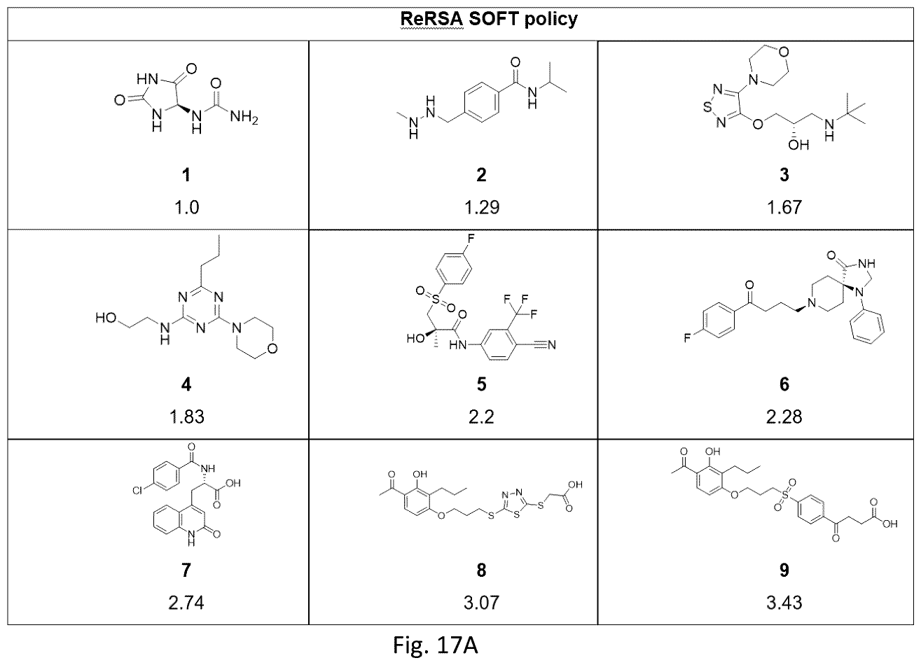

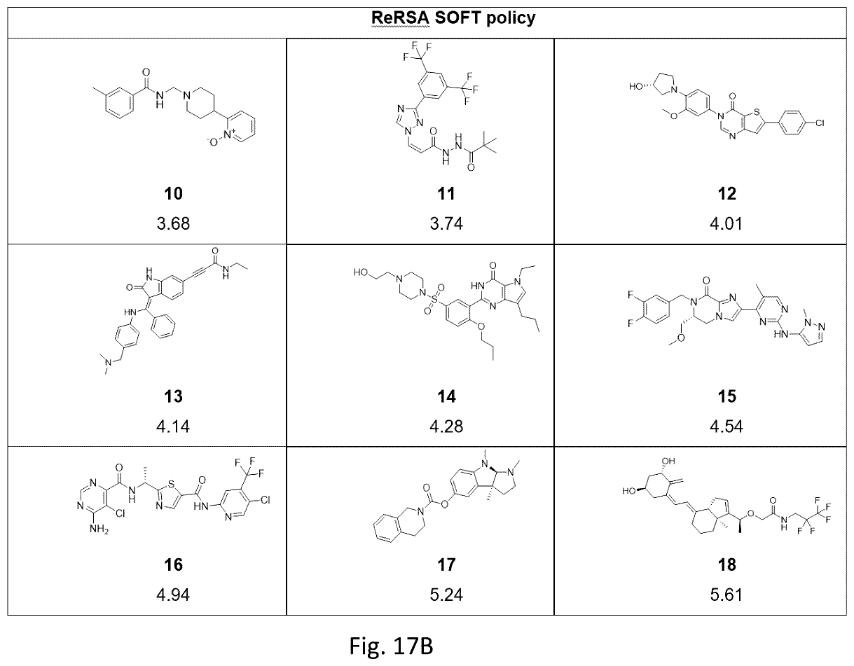

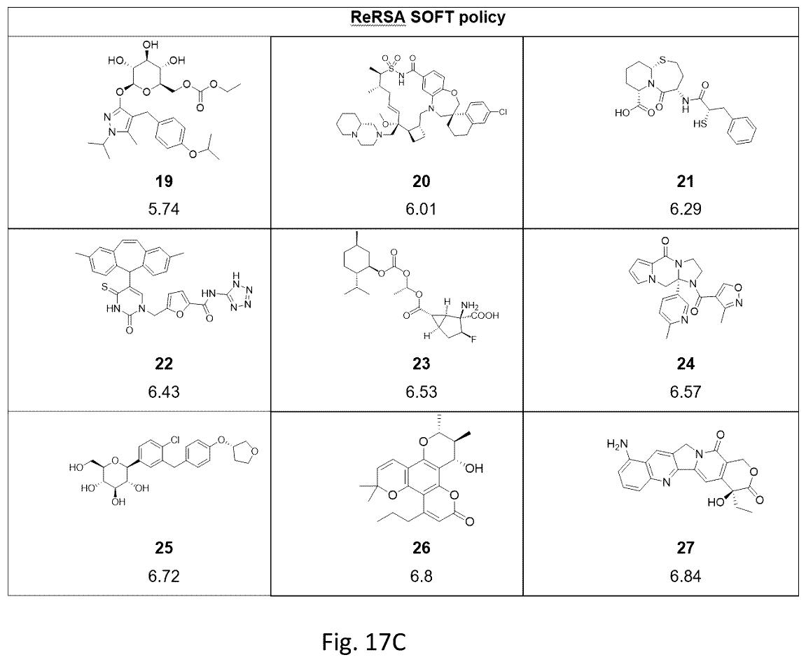

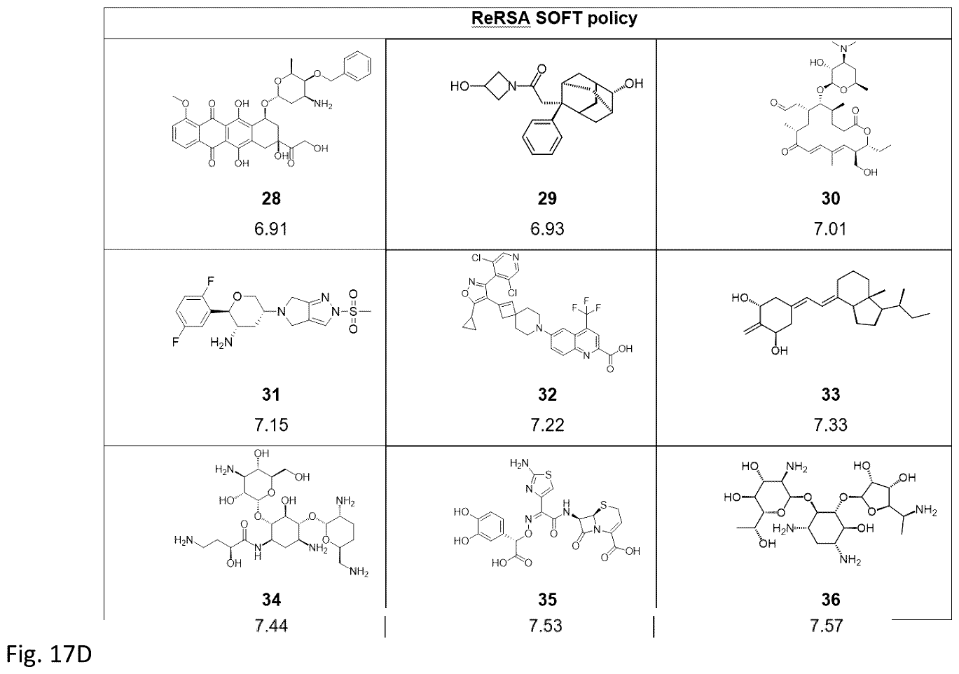

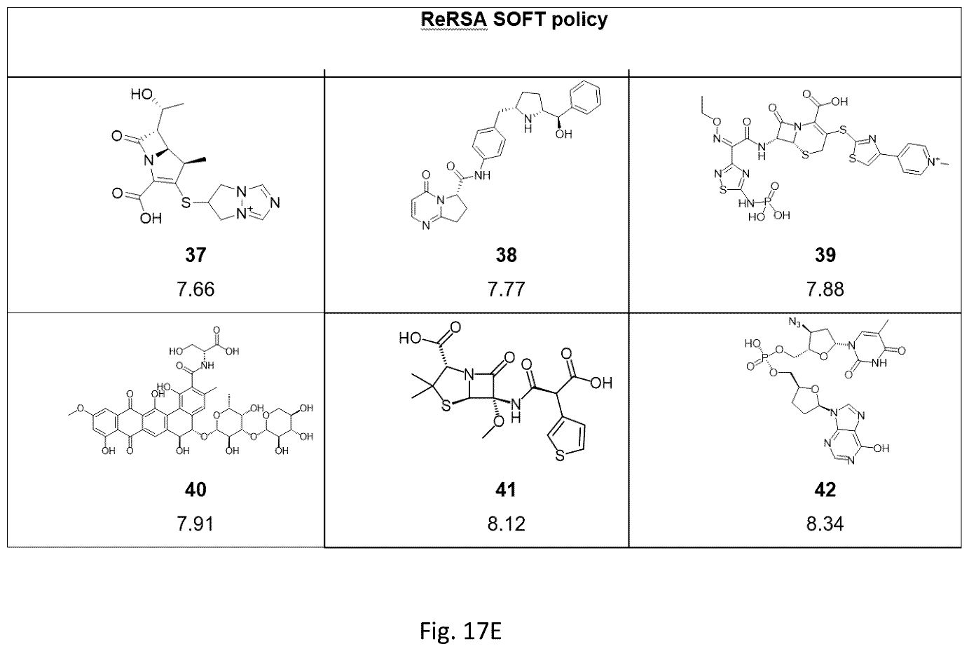

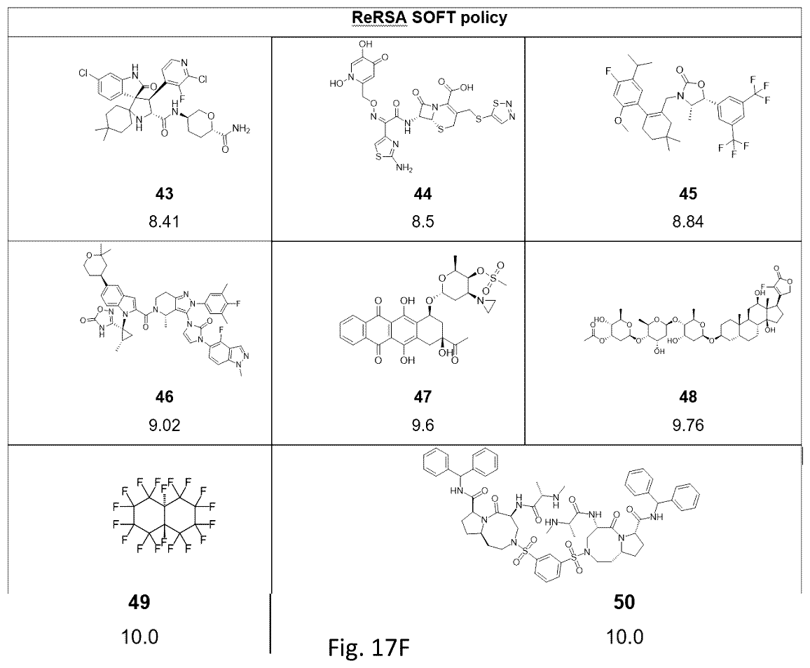

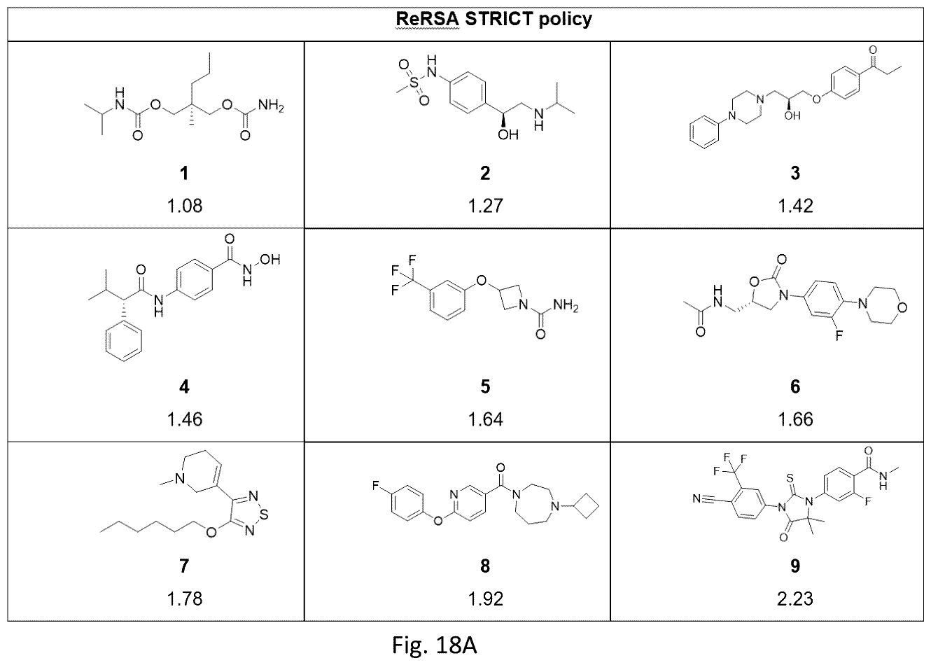

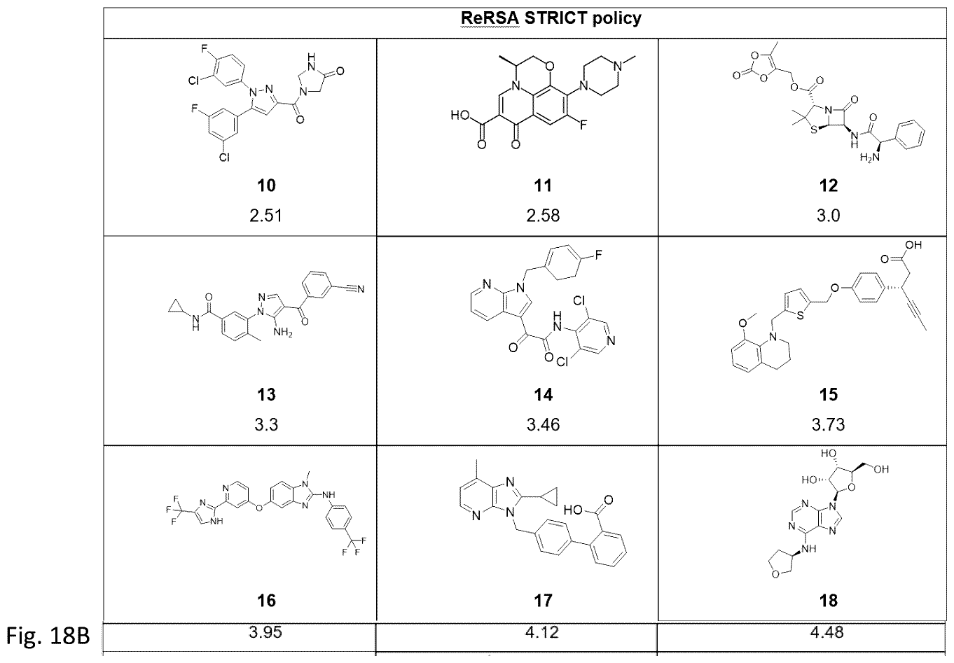

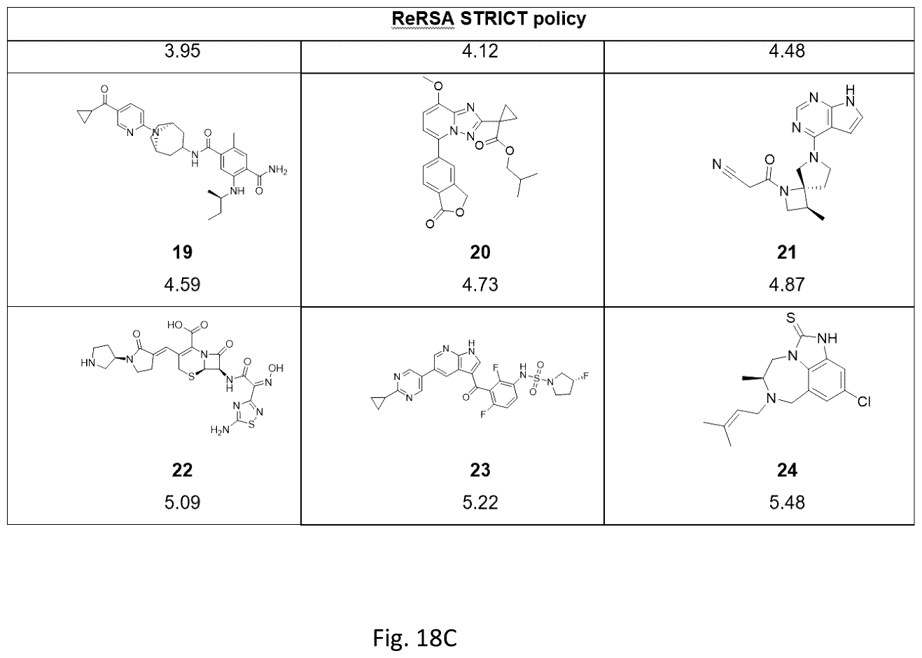

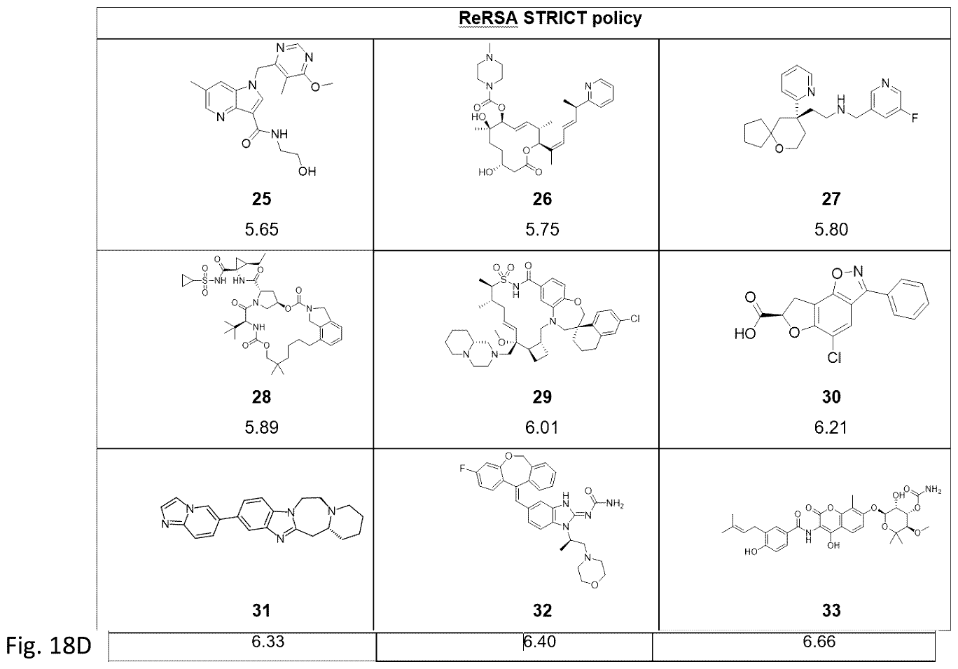

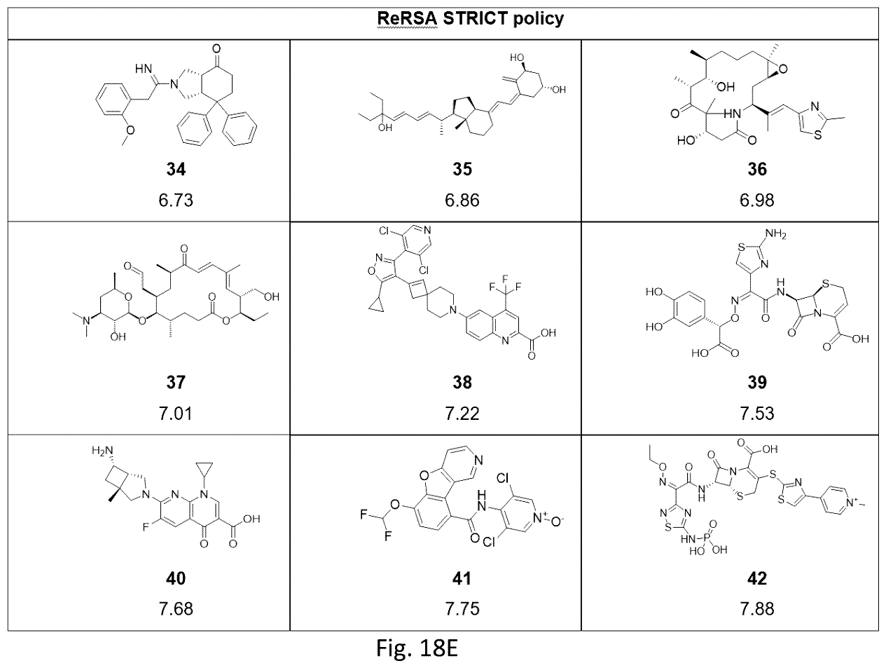

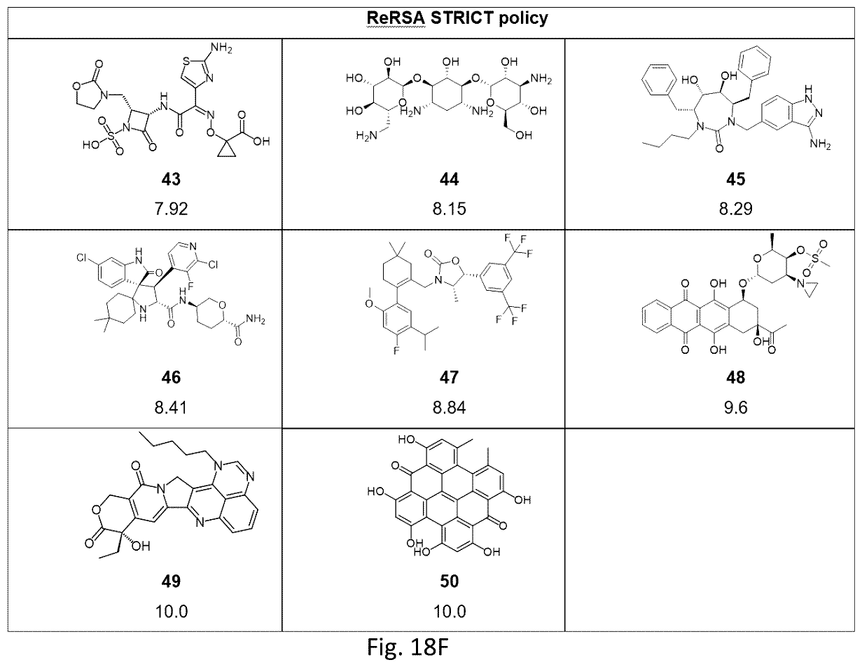

The FIGS. 17A-17F and FIGS. 18A-18F include examples that provide the evidence of how ReRSA scores fit the increasing molecular complexity in the given rows for both SOFT and STRICT policies.

### Experiment 5—ReRSA as a Measure of a Single Chemotype Local Alterations

The hit-expansion and hit/lead optimization campaigns utilize alterations in a molecular structure including but not limited to: Substituent insertion in ring(s); Linker insertion/elongation; Ring fusion; Spiro-point integration; Chiral center insertion; H-substitutions at heteroatoms (e.g., NH, OH); Bioisosteric replacements.

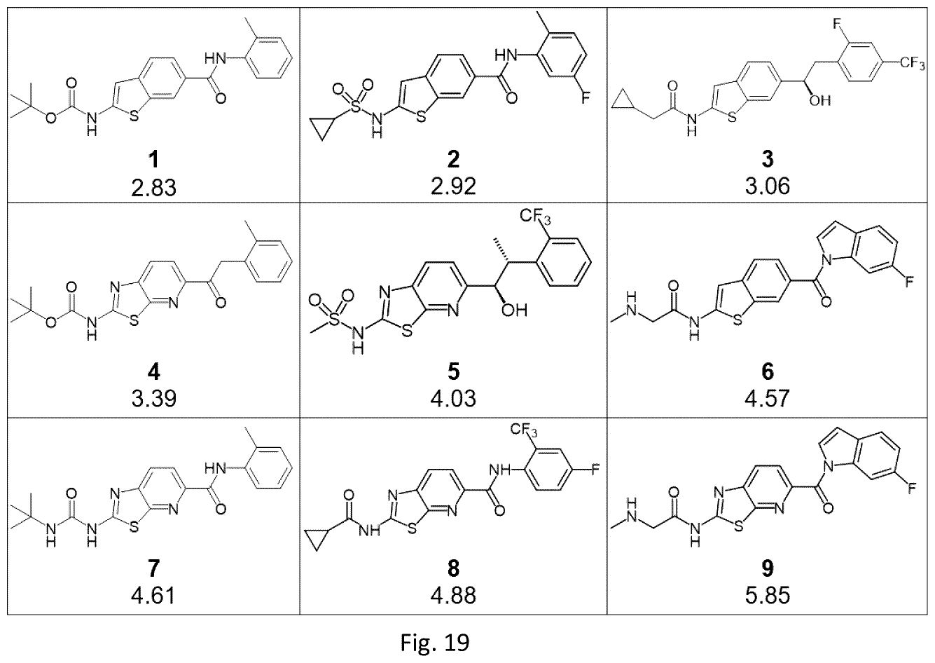

Also the design driven by a medicinal chemist or by generative models can provide alterations that can result in rare and underrepresented substructures. A good SA metric should behave as a proper navigation system to be applied for the optimization/expansion of a chemotype. FIG. 19 illustrates local alterations in a single chemotype are reflected in the corresponding ReRSA scores, with alterations of ReRSA 3.0 scores reflecting local alterations for a single chemotype.

### Experiment 6—ReRSA as a Tool to Balance Between Novelty and Actual Synthetic Accessibility

The generative chemistry pipelines can produce thousands of structures per generative launch and manual check in the reference databases/datasets (i.e., ChEMBL, Enamine or SciFinder) makes the procedure of structures nomination time-consuming. ReRSA algorithm includes Algorithm 4 that enables filtration of non-relevant SMARTS substructures with either low or no presence in the reference dataset when STRICT policy is turned on. This feature reduces the number of false positives (a structure predicted to be synthetically accessible, but the synthesis revealed to be unfeasible or not possible) significantly. However, at the same time rare or non-present substructures accumulate novelty and some of them can be potentially synthetically accessible in practice. If a medicinal chemist would like to prioritize novelty over the actual synthetic accessibility (e.g., in terms of comparing to an existing reference dataset of synthesized compounds), enabling SOFT policy will support their intentions. In this case an assessed structure will not be penalized, if a non-reported substructure is a part of the whole structure. FIG. 10 illustrates examples for structures accessed by ReRSA in both SOFT and STRICT policies. 5-membered substituted heterocycles are considered as ‘Achilles' heel’ due to their diversity and checking whether a particular heterocycles has been reported so far or not represents a time-consuming procedure. Non-relevant substructures are highlighted and their presence is taken into account when STRICT policy is enabled in order to ultimately penalize the accessed structures, making ReRSA score 10 for them. On the other hand, the SOFT policy provides a non-critical penalty for such structures. It can be beneficial for the cases, when the remaining parts of a structure are not unfeasible and both synthetic challenges and novelty are accumulated in the highlighted region. FIG. 10 shows rare or non-reported substituted 5-membered heterocycles highlighted at the de novo generated structures accessed with two ReRSA 3.0 scoring policies (SOFT and STRICT).

### Experiment 7—SYBA Dataset

SYBA is one of the ways to model SA by using a Bayesian naive classification method applied to the training data set of hard-to-synthesize (HS) and easy-to-synthesize (ES) molecules. As for HS test set part (T_{CP-}) 3581 molecules have been extracted from the GDB-17 database if complexity indices exceed thresholds. Complementary number of ES molecules have been extracted from the ZINC15 database ensuring that the complexity indices do not exceed the same thresholds (T_{CP+}).

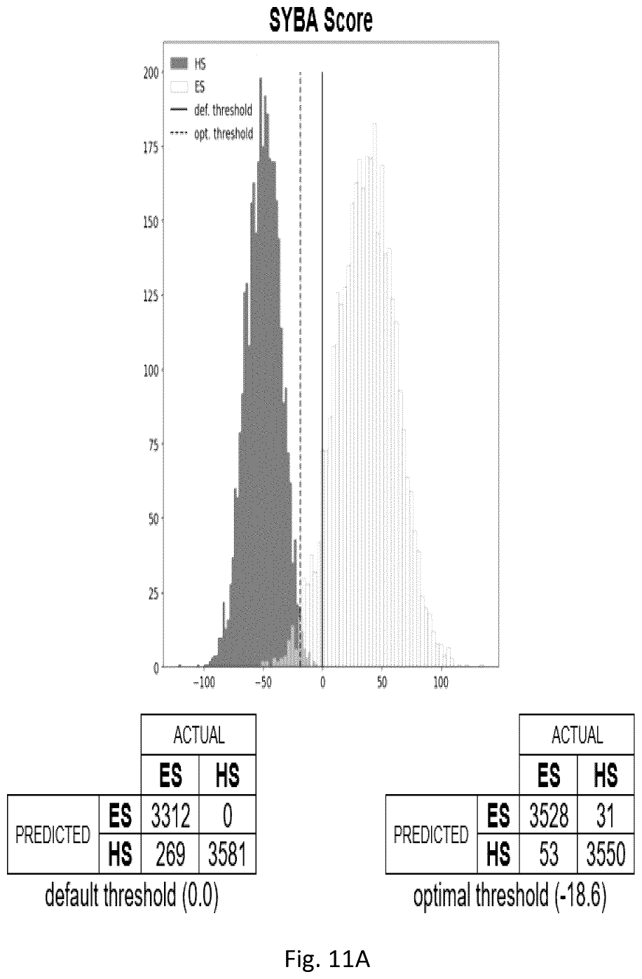

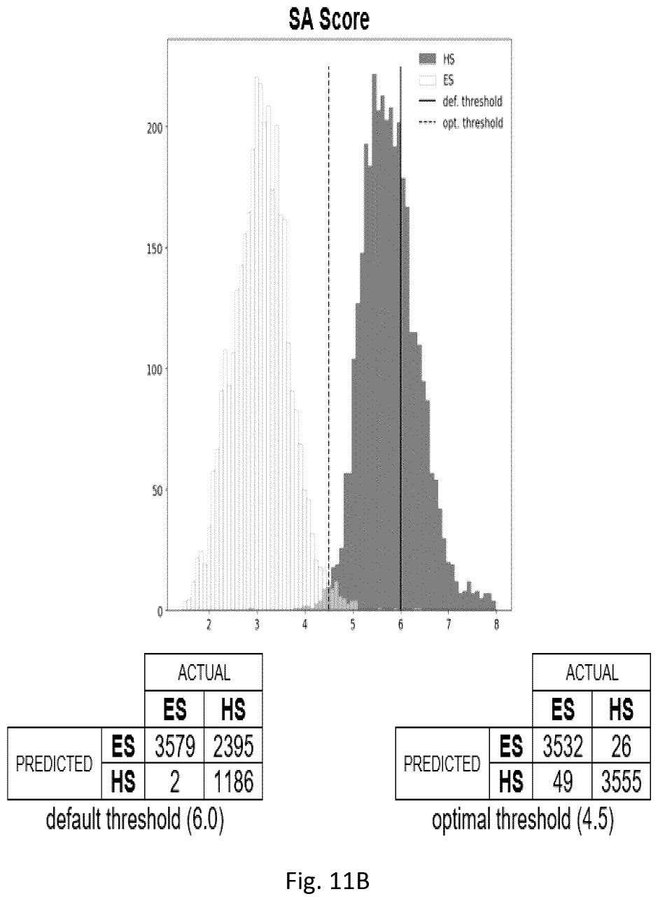

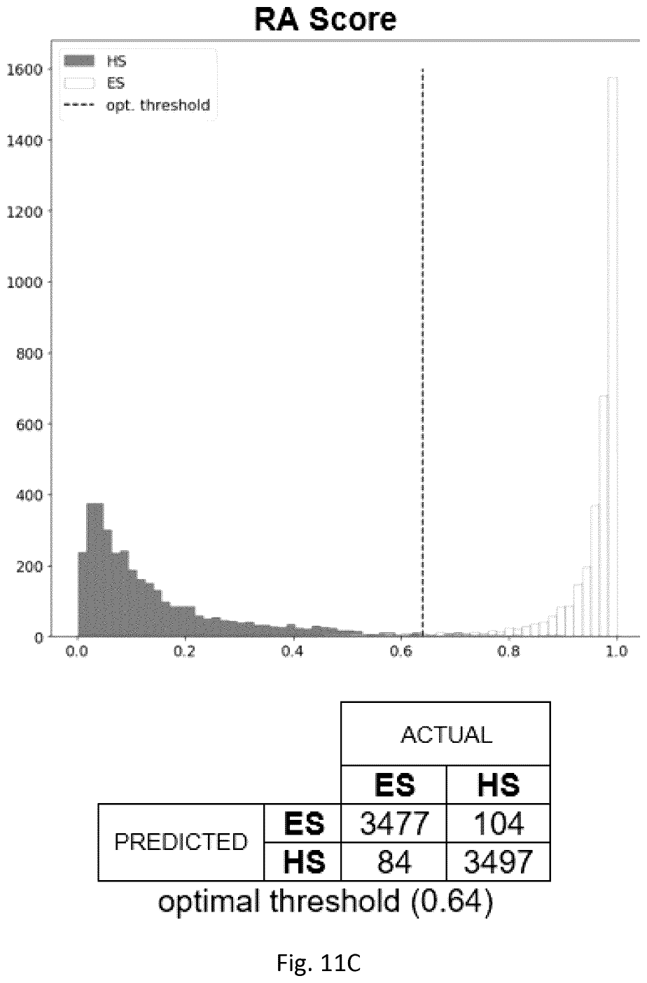

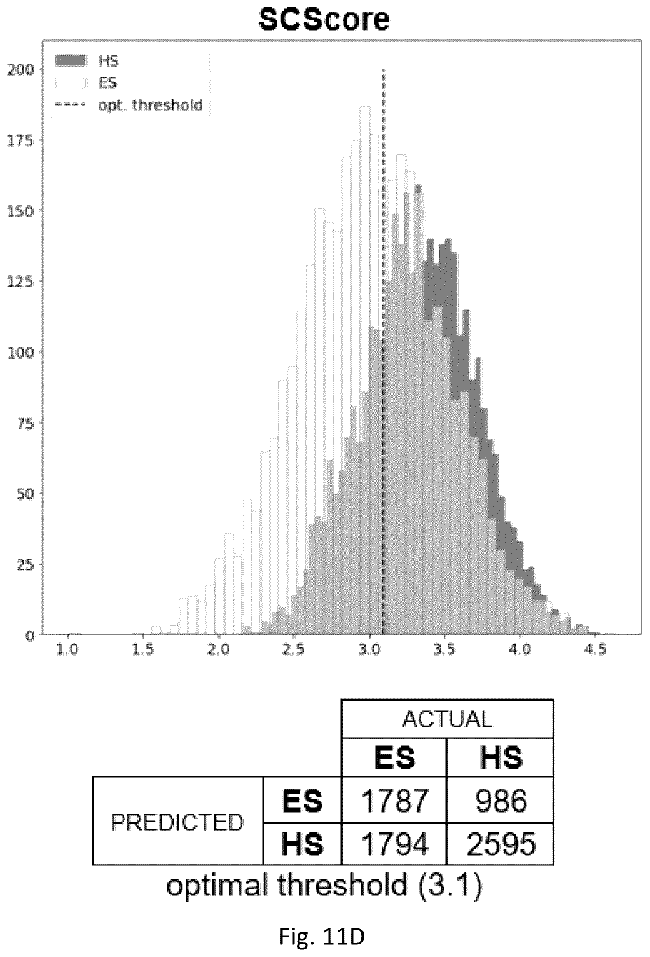

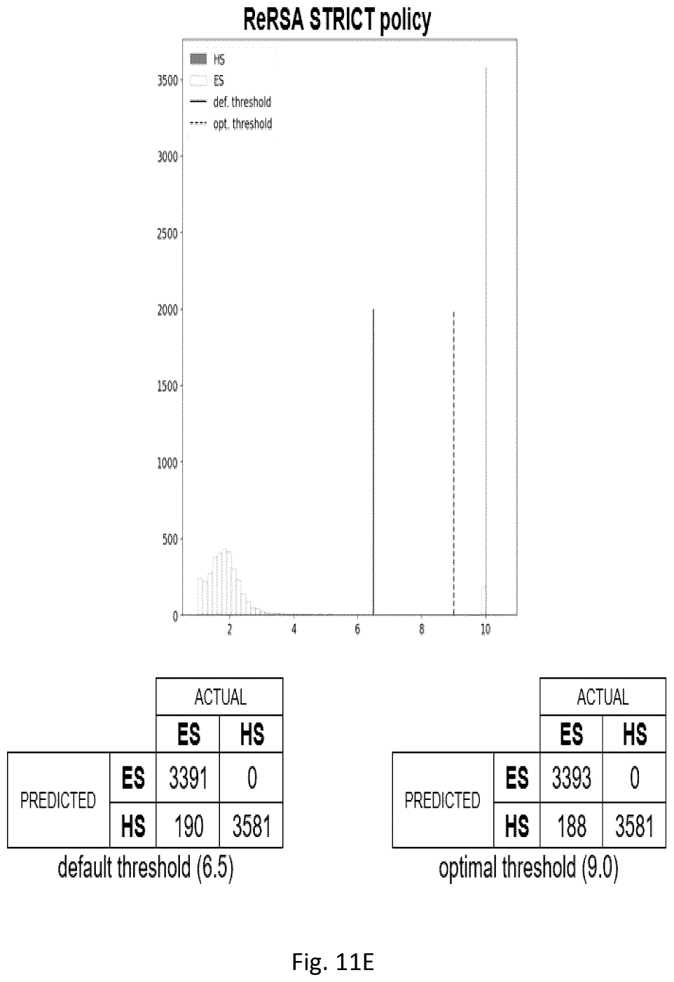

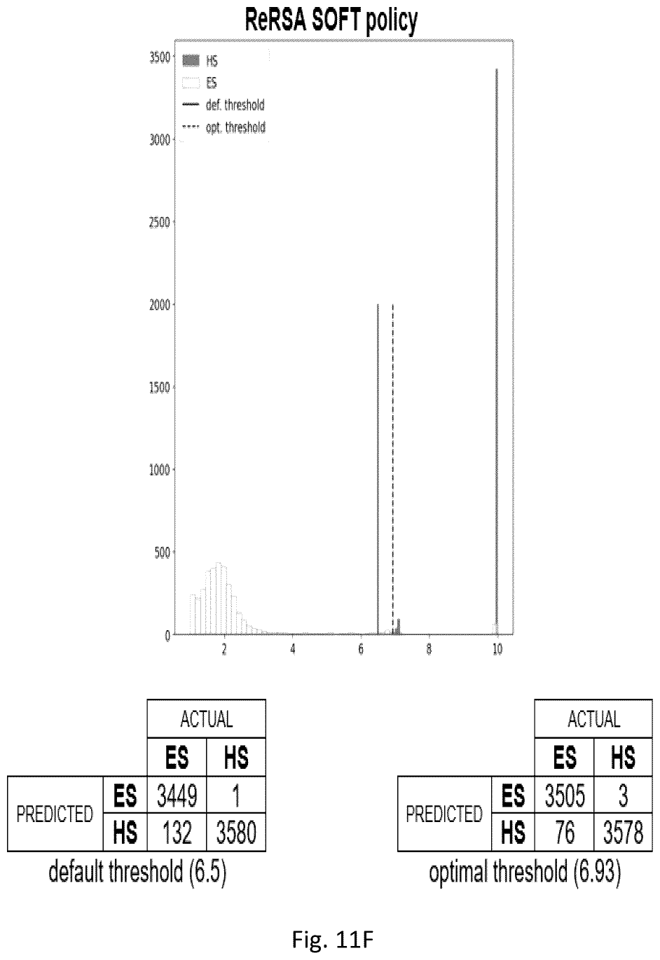

The ability of different SA modeling methods to discriminate between HS and ES molecules from the T_{CP} SYBA dataset was examined. The comparison is provided below for SYBA score, SA Score, RA Score, SCScore and two scores from the ReRSA method for both SOFT and STRICT policies (see FIG. 11A-11F). If discrimination threshold was given in the research article/patent describing method, the discrimination ability was assessed using the given metric threshold value (default threshold). At the same time in order to be not biased or in the case if the default threshold is not provided by the authors the optimal thresholds (minimum of {FN+FP}, FN—false negatives (false HS), FP—false positives (false ES)) were calculated for all SA modeling methods.

For the default thresholds, the ReRSA SOFT policy SA modeling method provides the most prominent discrimination power with 133 of {FN+FP} value. At the same time SCScore is not capable of discriminating HS from ES even at the optimal threshold, since the recommended threshold was not given. Interestingly, even SYBA was not able to beat ReRSA at the default threshold with {FN+FP} value of 269. This can be explained by the fact that the authors of SYBA were more interested in the minimization of FP, which can be considered as a wise strategy applying to real generative chemistry experiments. Thus, SYBA provides an excellent result of FP=0 at the default threshold similarly to ReRSA STRICT policy SA modeling method (FP=0) and ReRSA SOFT policy (FP=1).

For the optimal thresholds, ReRSA SOFT policy is slightly better than SYBA in terms of {FN+FP} values (79 vs 84), while the best {FN+FP} value of 75 is demonstrated by SA Score at its optimal threshold of 4.5 which is different from the recommended one (6.0). However, both ReRSA policies are still capable of outperforming SA Score to minimize false positives—26 for SA Score against 0 and 3 for ReRSA SOFT and STRICT policies respectively. FIGS. 11A-11F illustrate the comparison of discrimination power of SA modeling approaches on T_{CP} SYBA test dataset.

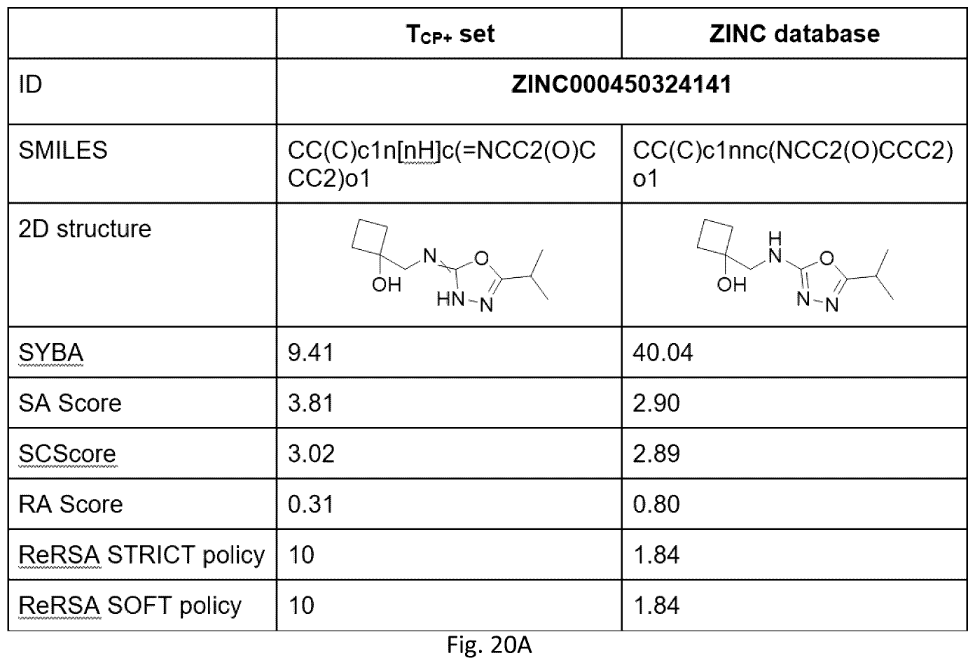

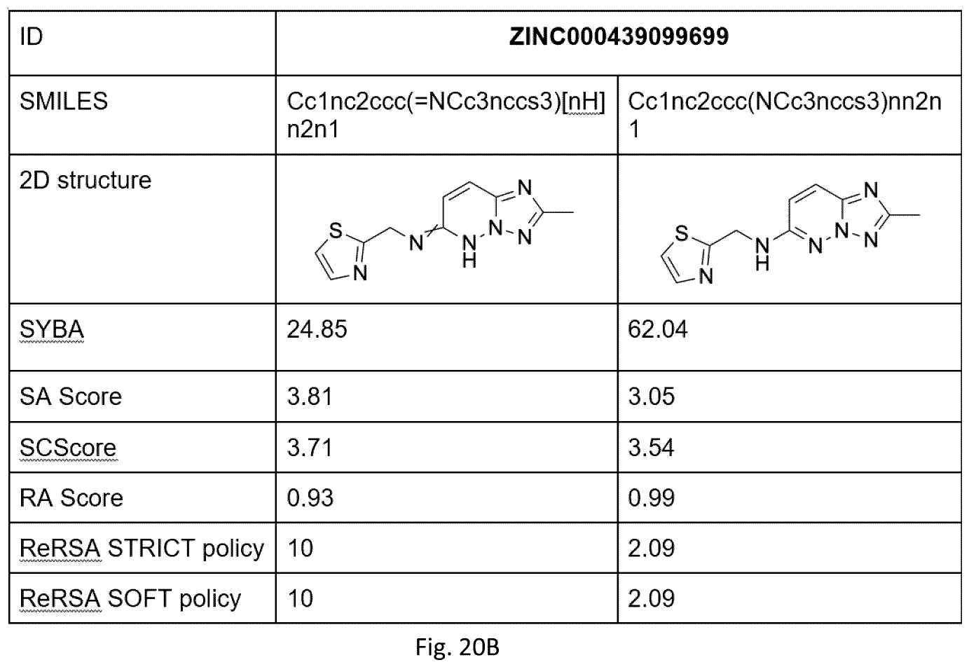

First, it should be noted that the original molecules from ZINC can be different from those from TCP+ test set due to inconsistencies of tautomer forms. For example, ZINC000450324141 and ZINC000439099699 are provided in TCP+ test set and are presented as different tautomers from those provided in ZINC (see FIGS. 20A and 20B). More importantly, the selected tautomer form affects the SA estimation values significantly for all assessed SA modeling methods.

Since none of the tested algorithms have a procedure for selecting the correct (or more stable) tautomer, such errors in SMILES-strings can lead to false negative outcomes.

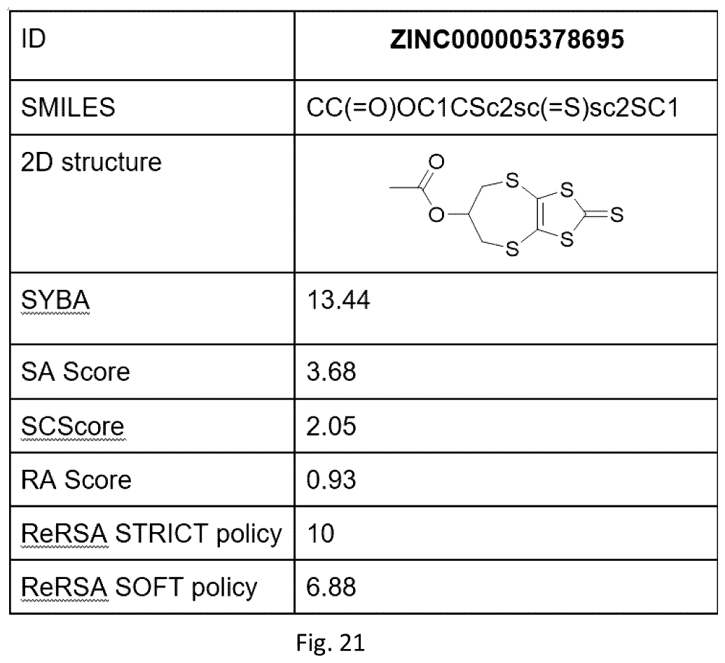

Secondly, the relevance of the ES dataset (T_{CP+} set) is questionable since there are some molecular structures that are beyond the drug-like space (e.g., see ZINC000005378695 in the FIG. 21), which is one of the most demanded and desired chemical spaces. For sure, SA is a property that ideally should be independent from the specification of chemical space, however, the possibility of getting a universal method of SA estimation is still considered as unachievable and thus focusing into targeted chemical spaces and modeling SA in the same task-specific manner is considered more reasonable and practical.

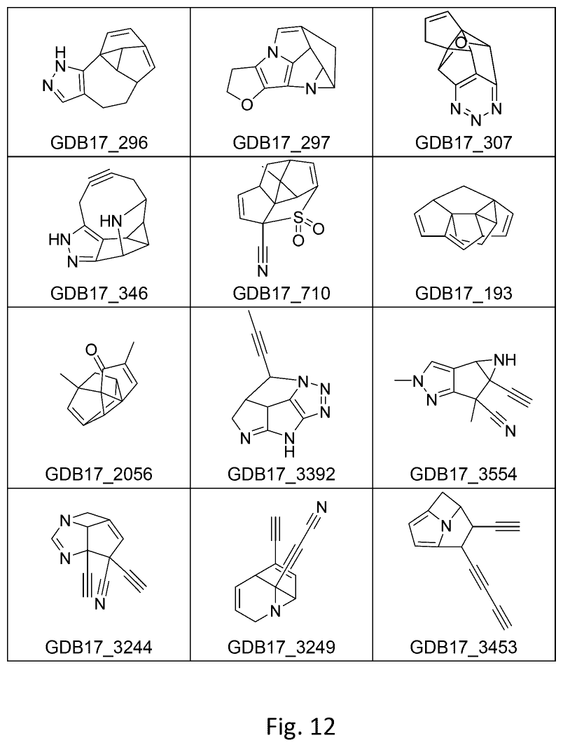

Besides this, a contentious aspect is that the selected T_{CP-} set represents an extreme percentile of the undoubtedly inaccessible chemical space (see FIG. 12), while the inaccessible space is significantly larger itself. Furthermore, the most intriguing part of the inaccessible chemical space appears in such a way that an average synthetic chemist should hesitate before immediately labeling it as inaccessible. Thus, a better T_{CP-} should be organized in the way to contain de facto inaccessible structures with human-wise non-certain and disputable synthetic accessibility status. FIG. 12 illustrates randomly selected structures from T_{CP-} set.

### Experiment 8—Expert Classification Test Set

To compare the most popular SA modeling approaches with ReRSA, a classical experiment with comparison of synthetic chemists' opinions and SC estimation for selected molecular structures as performed. In order to keep the context of SA modeling goals, the experiment used a dataset containing both registered drugs and molecular structures generated by generative models. We suggested experts provide binary classification into easy (ES) and hard (HS) to synthesize structures rather than SA quantification. To avoid bias of individual experts, we also removed from the final dataset all those structures for which at least one expert gave a different label from the others.

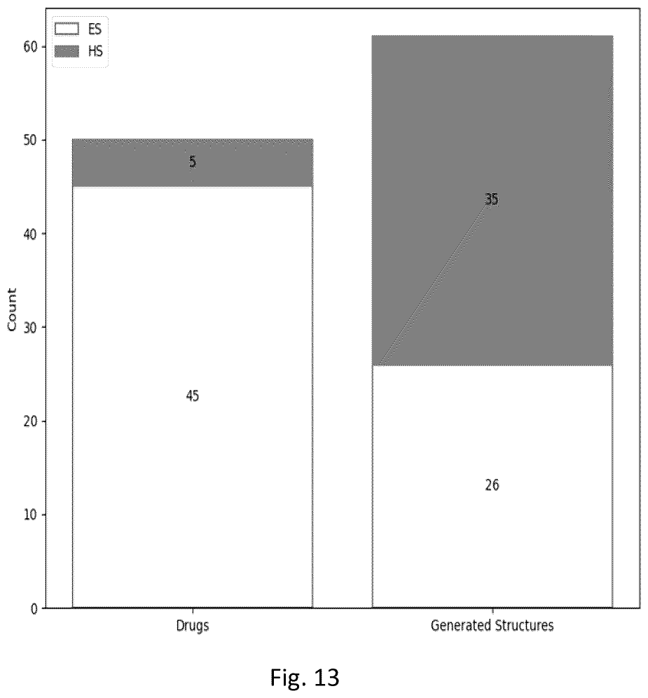

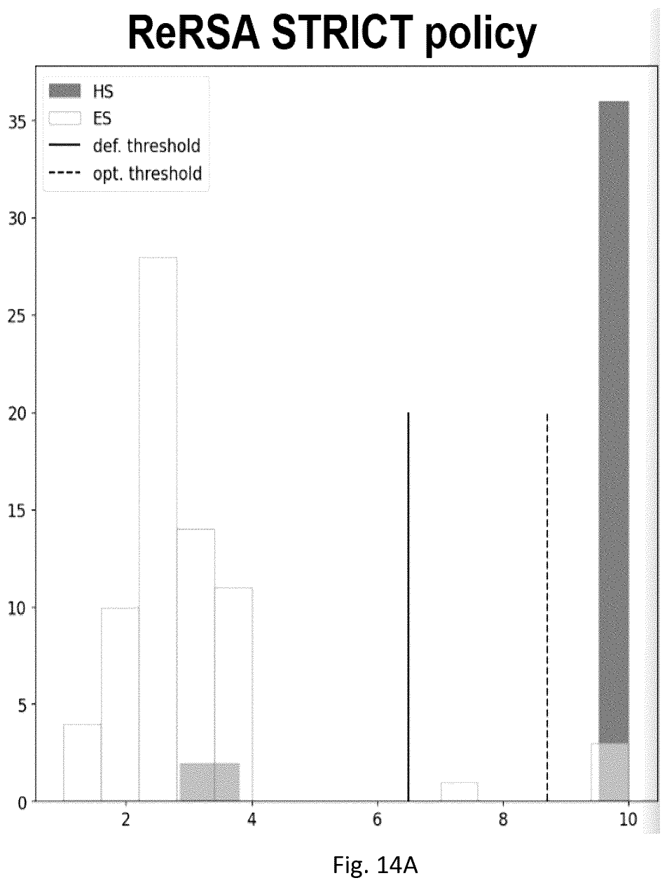

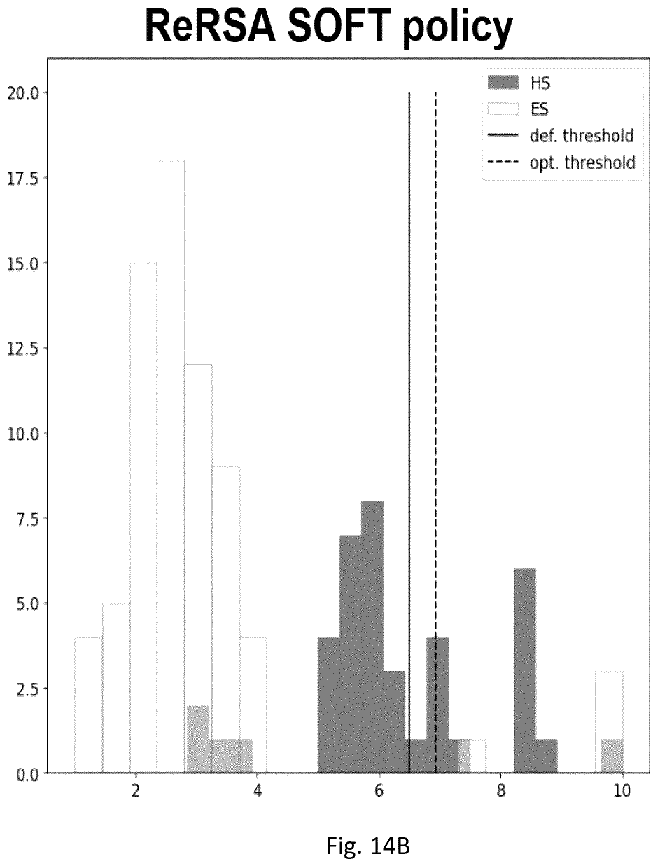

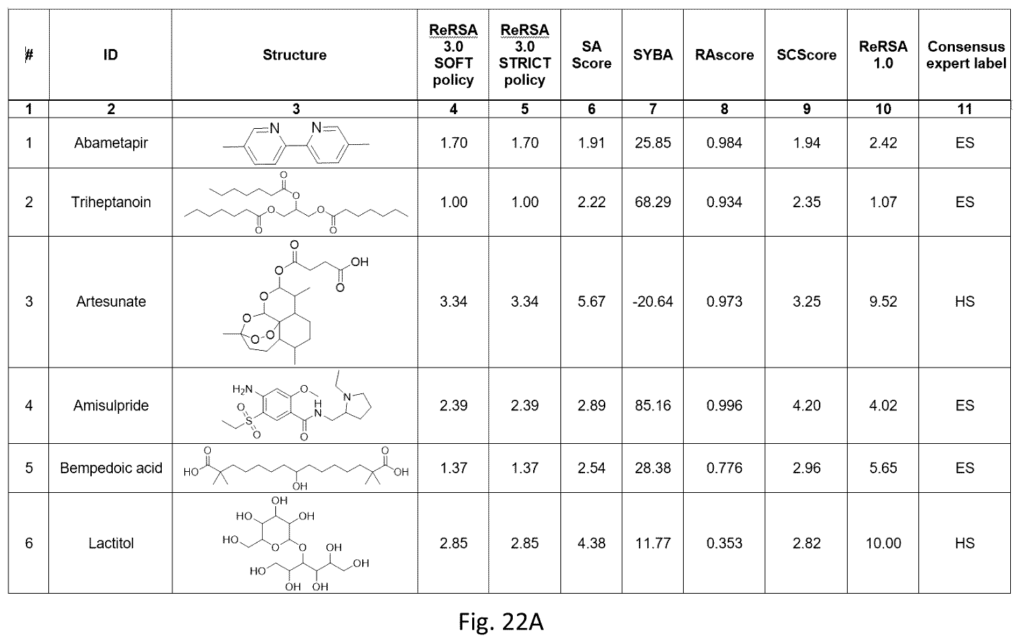

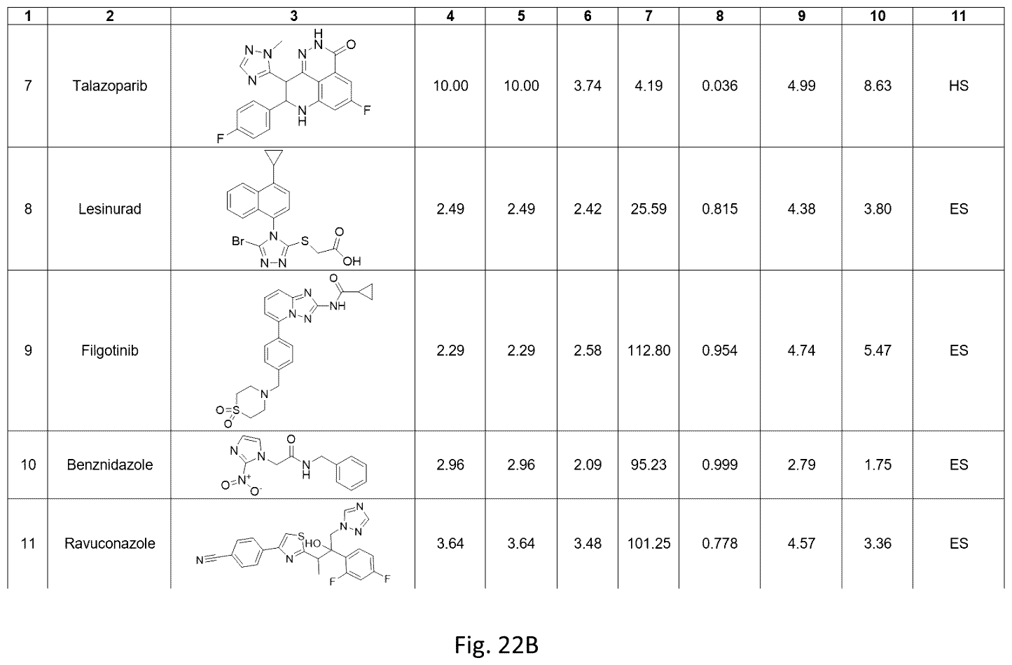

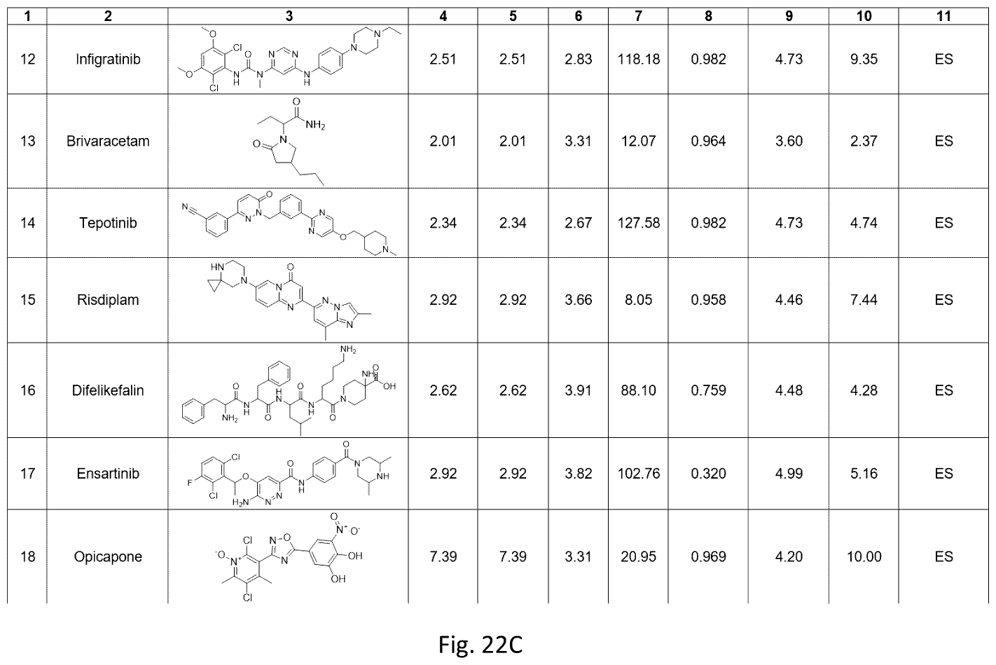

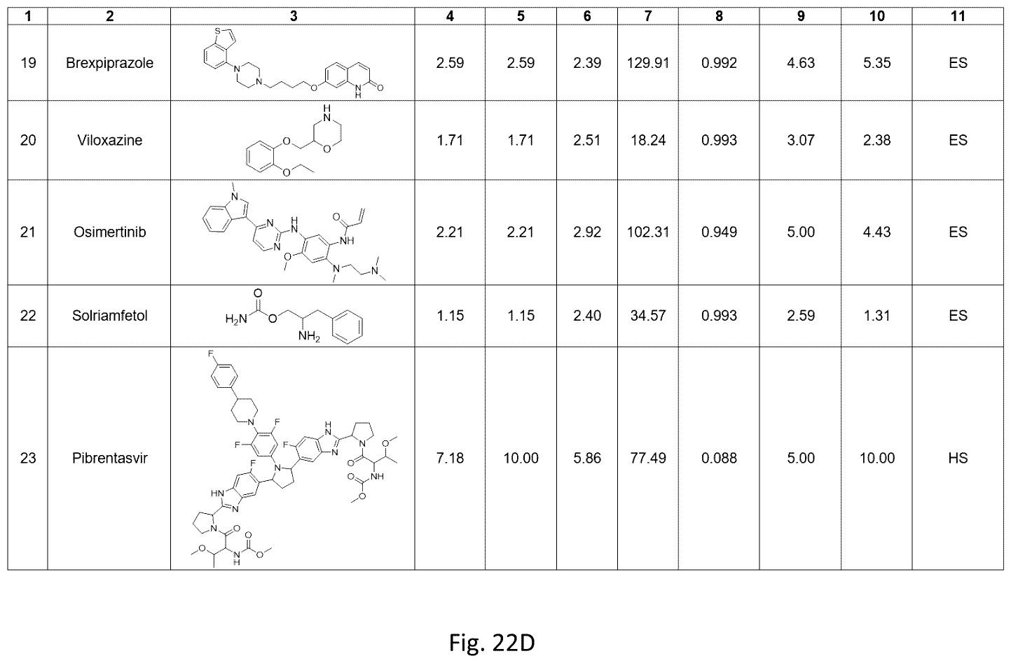

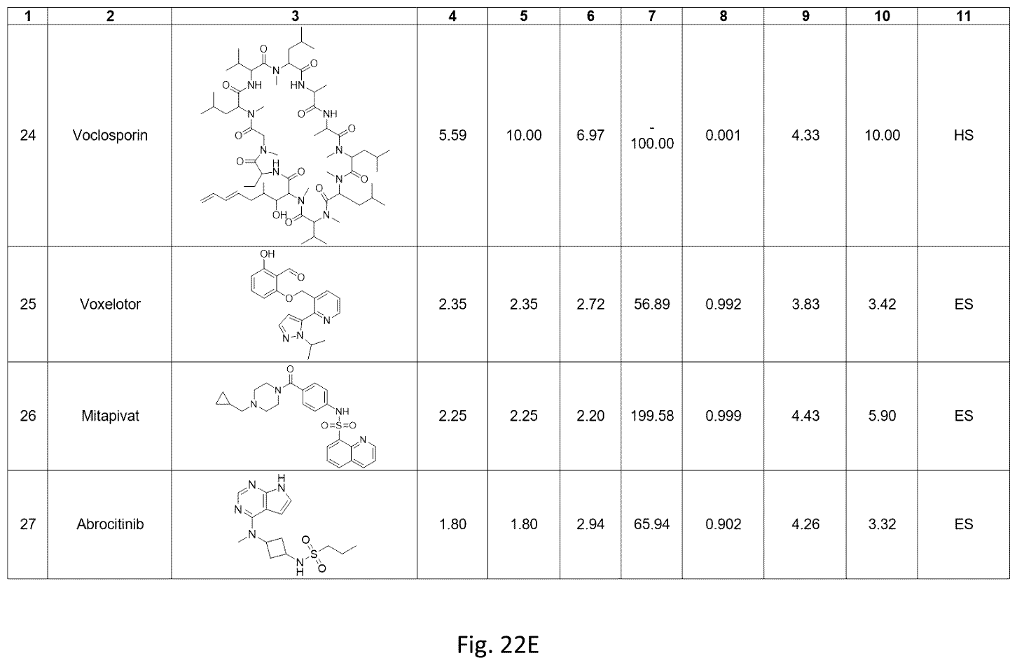

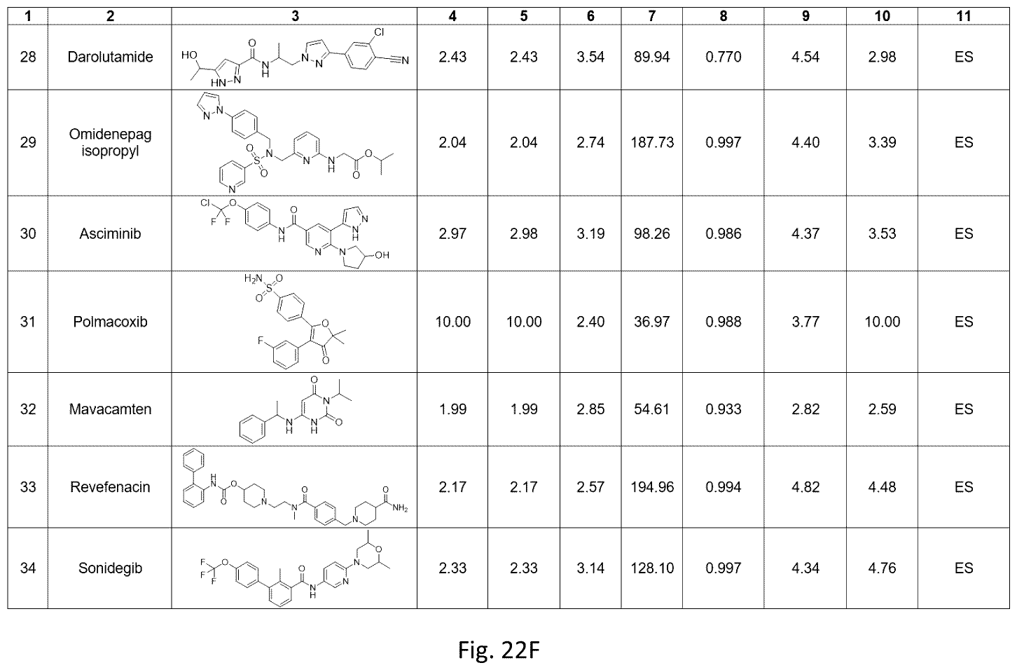

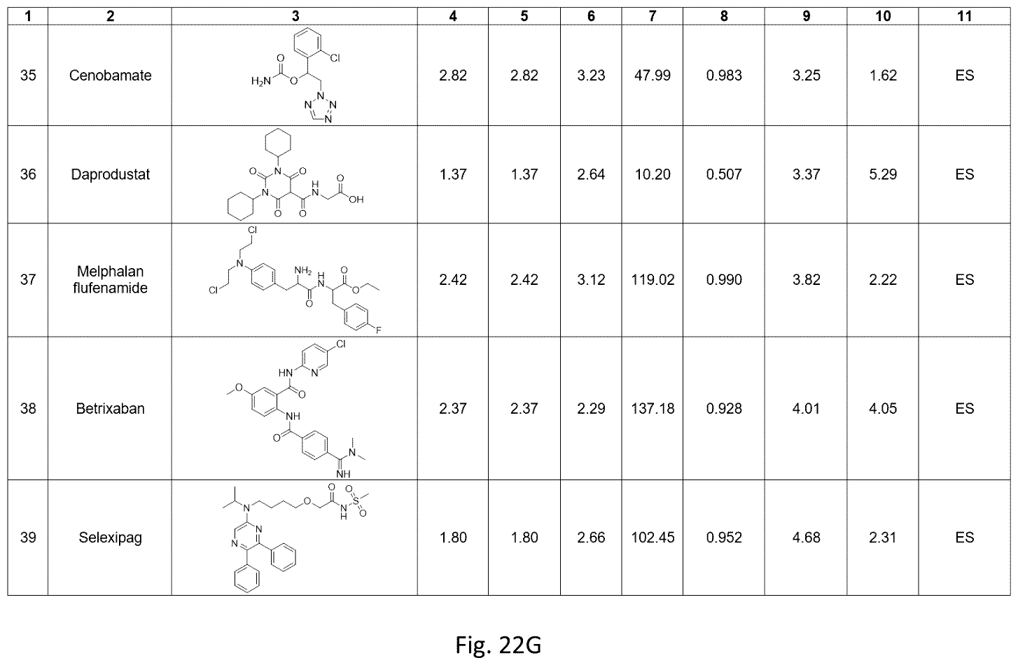

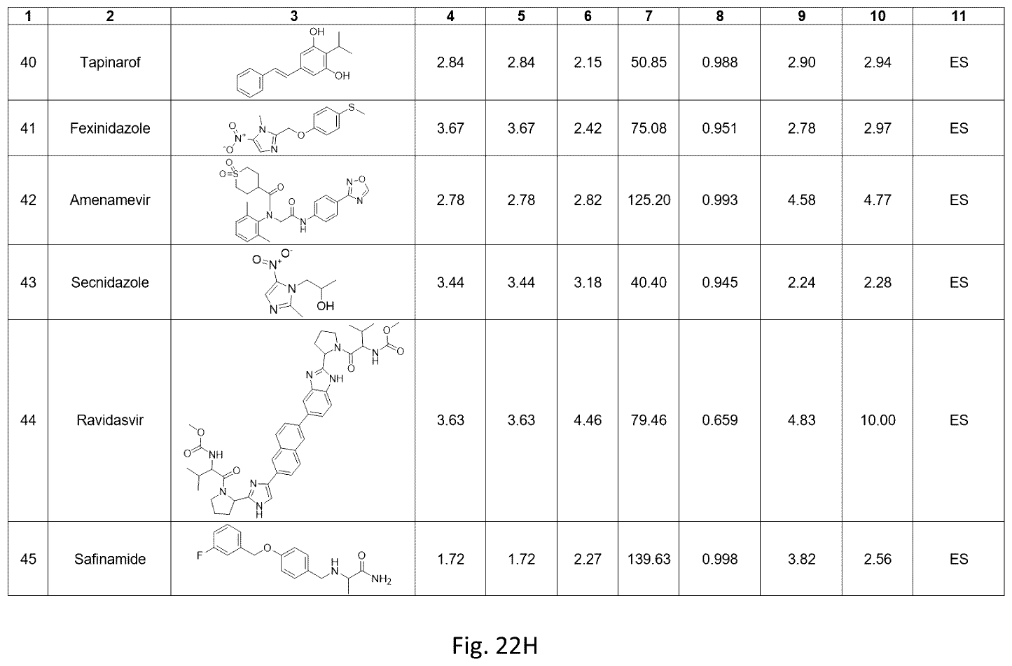

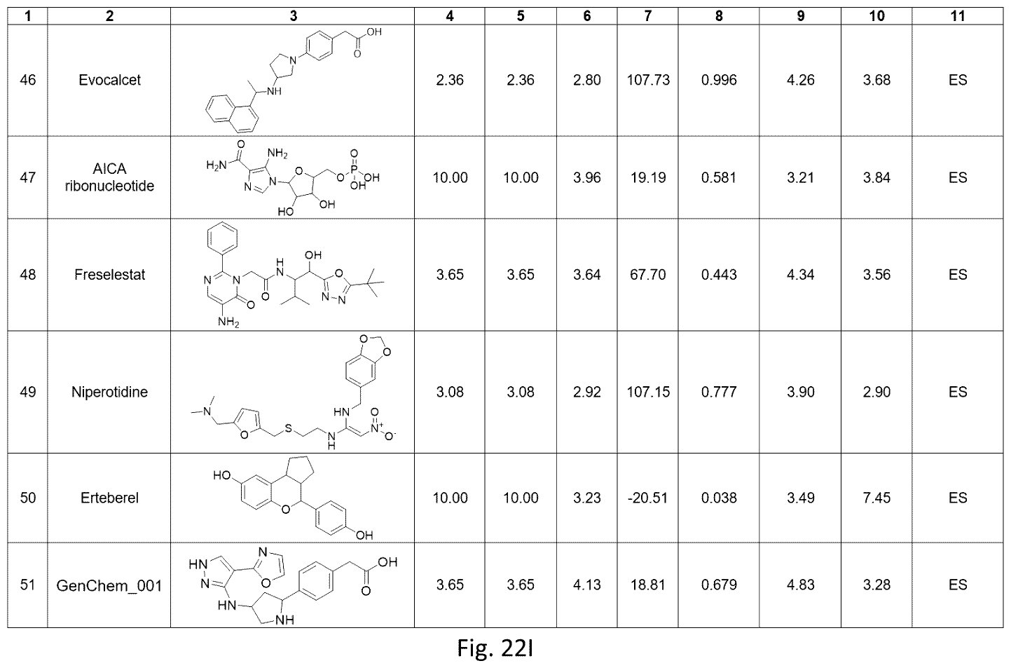

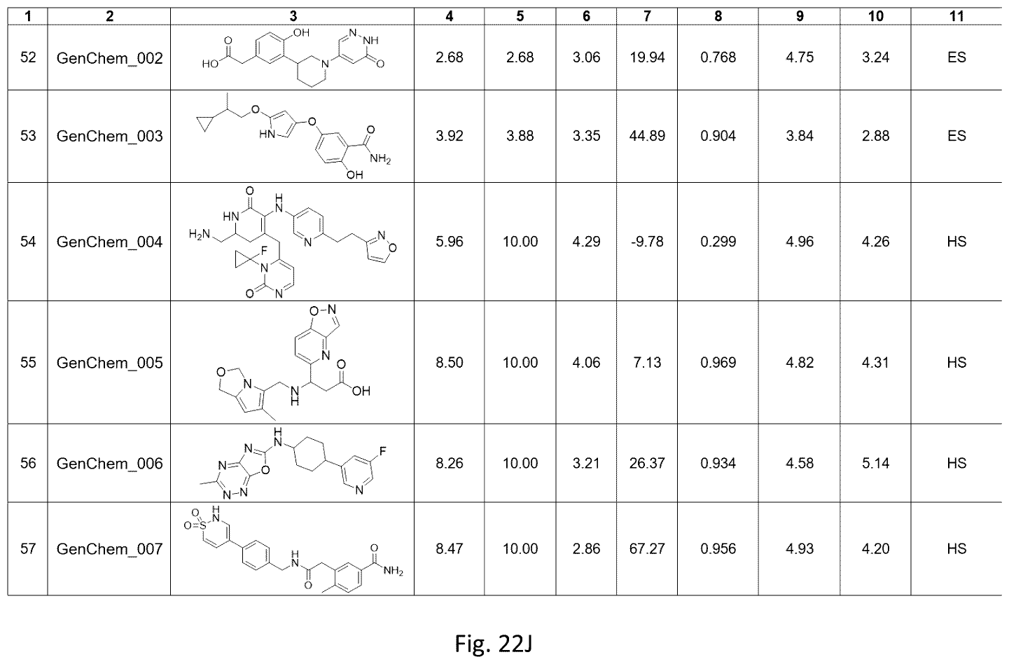

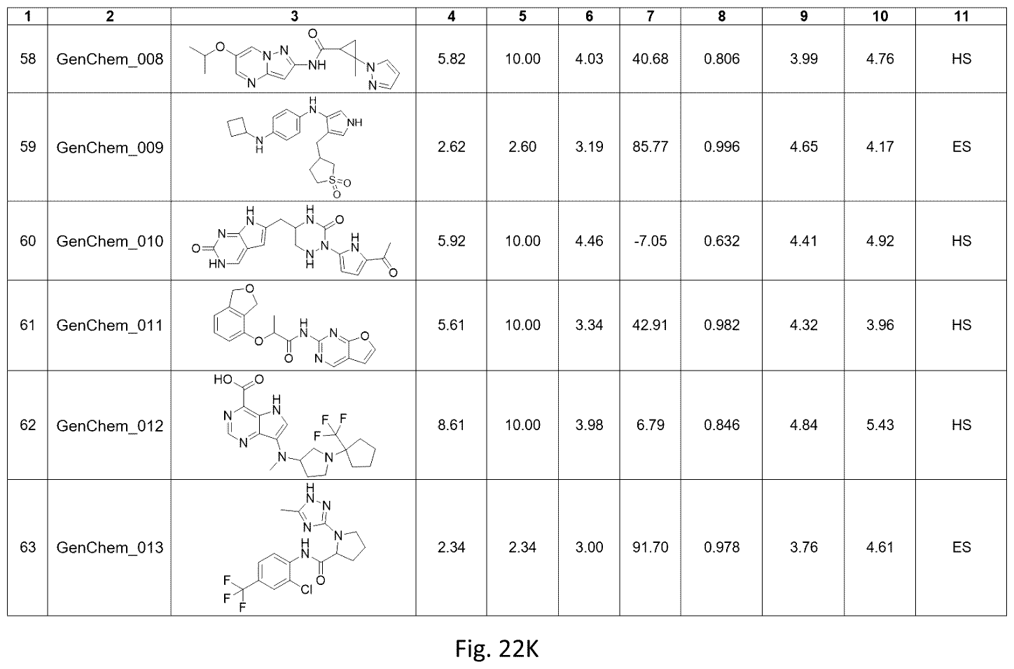

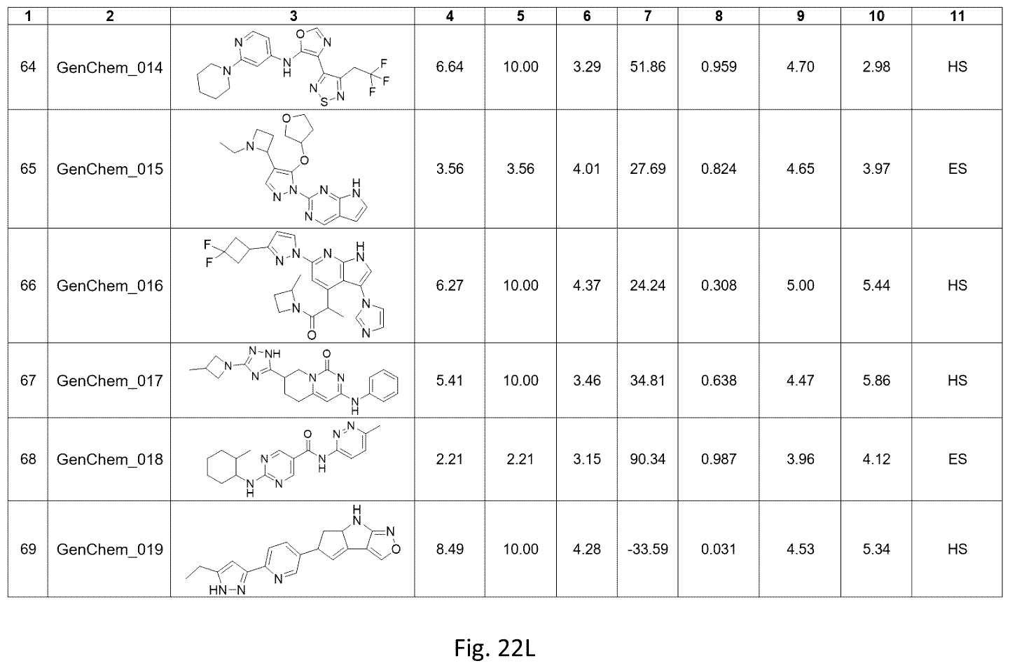

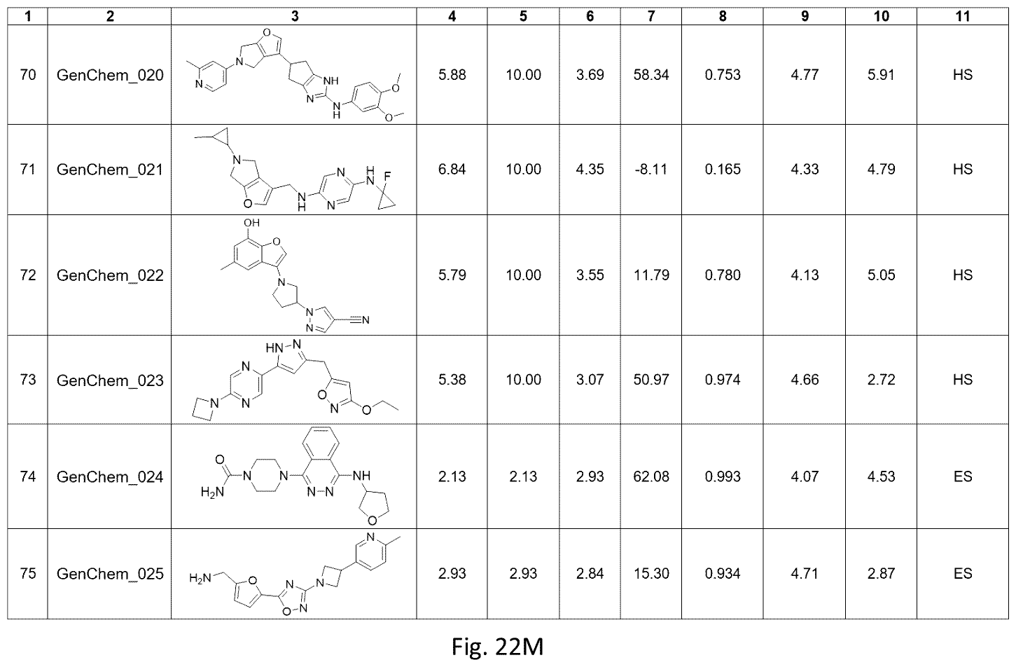

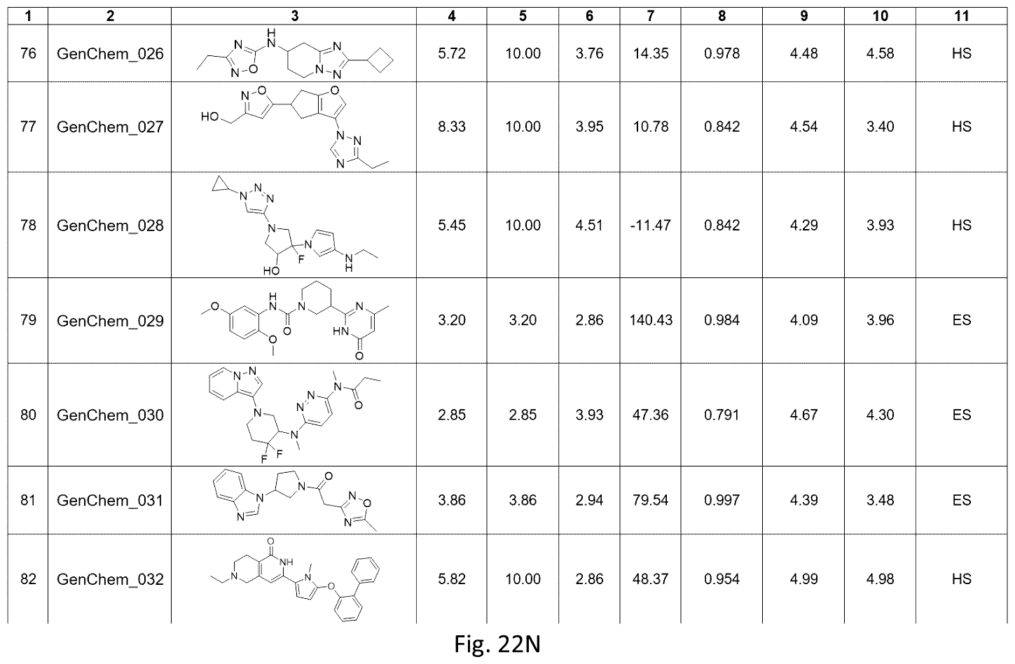

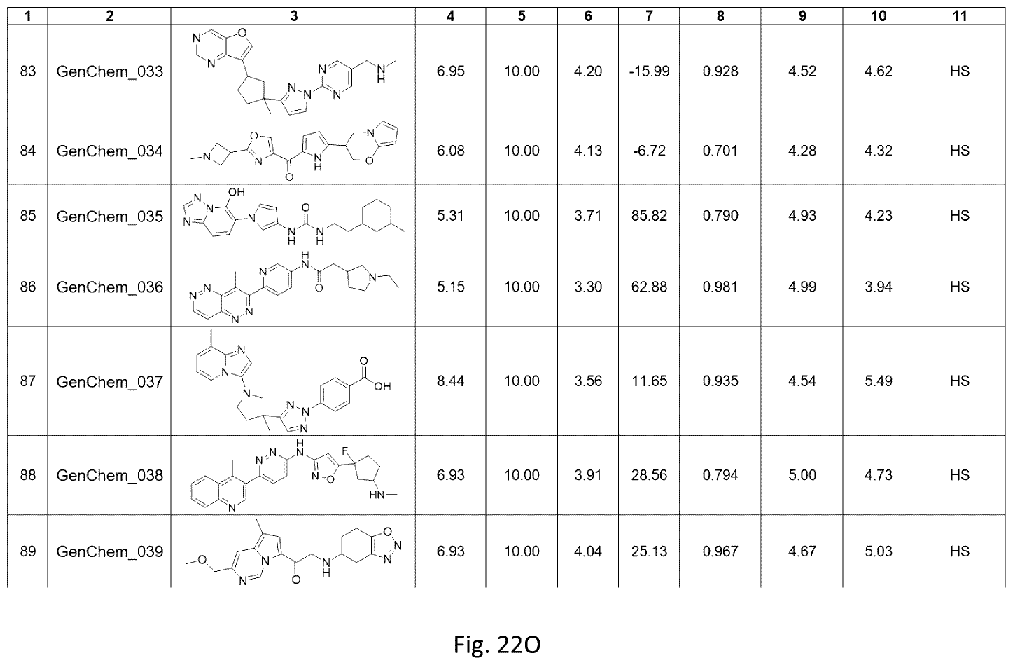

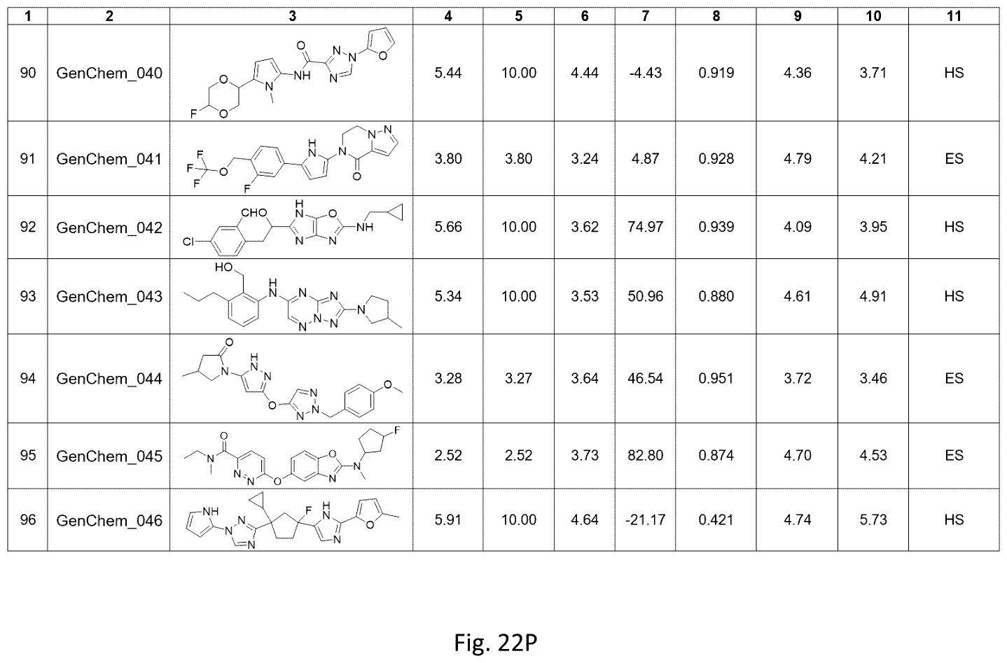

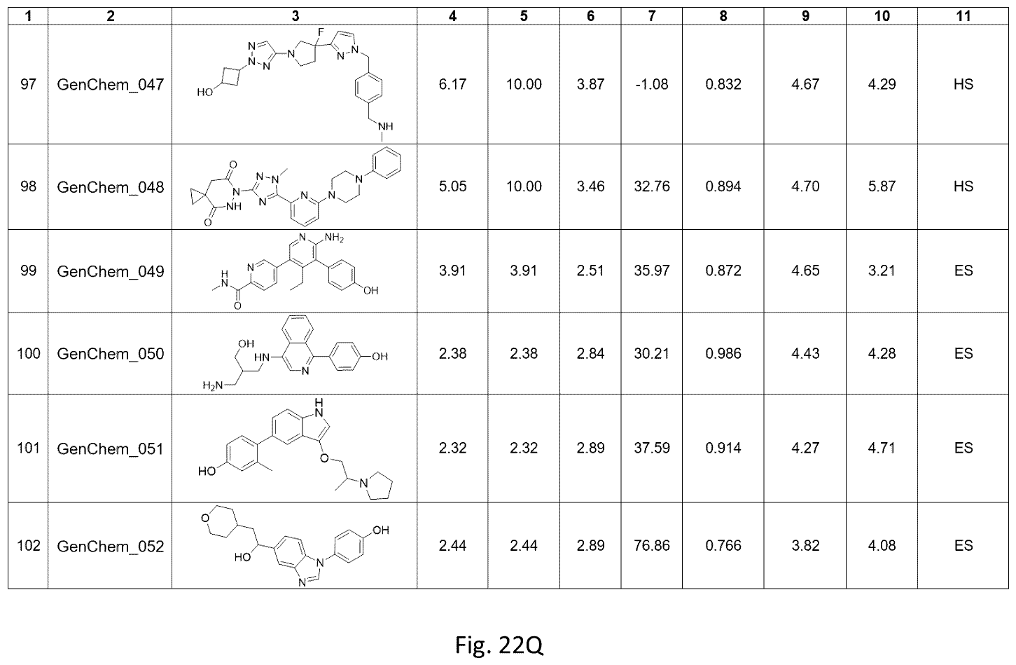

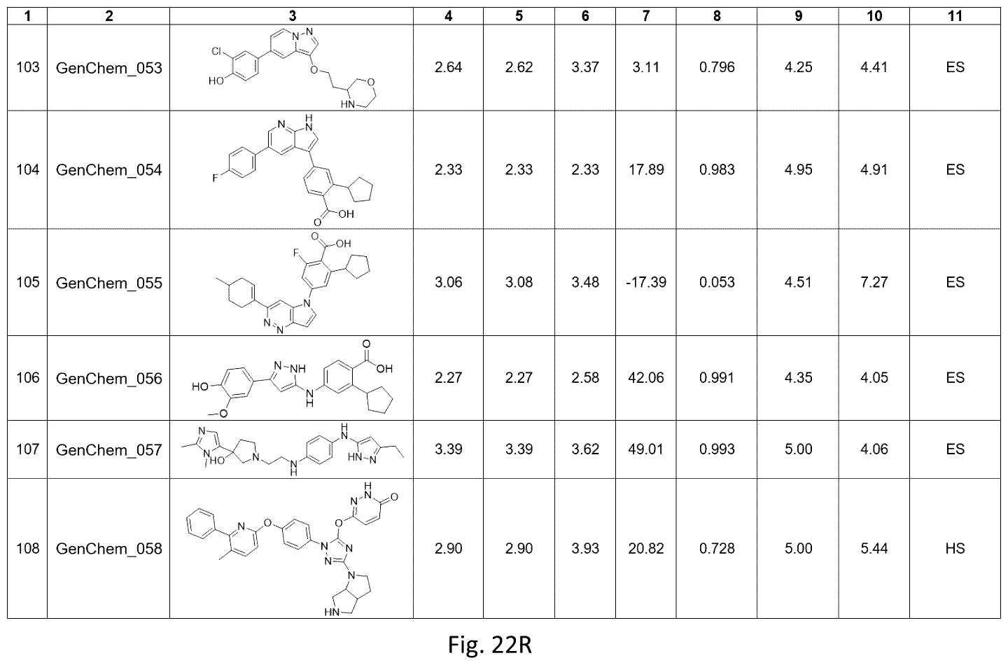

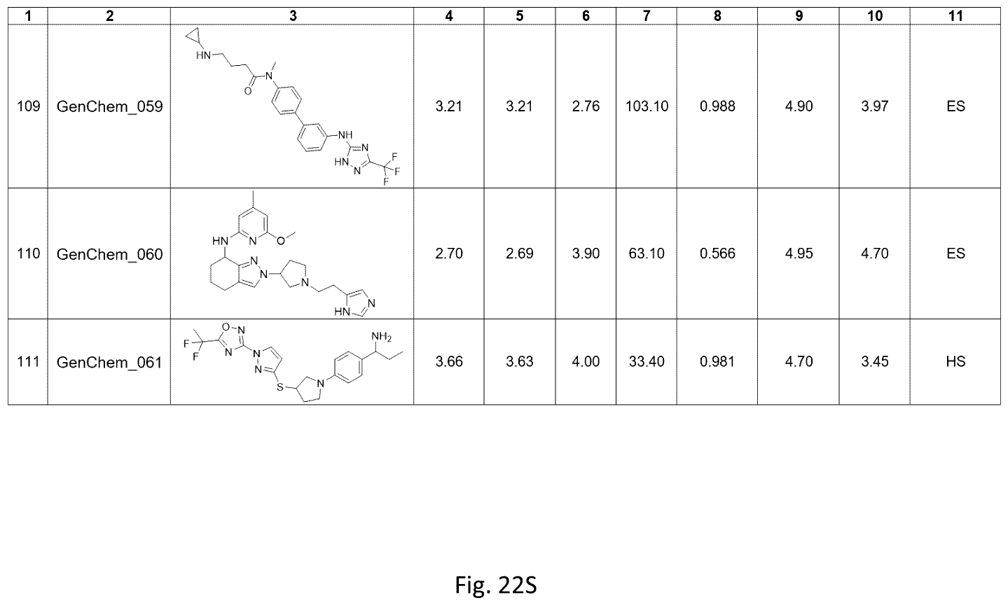

The initial set of 300 molecular structures includes 150 structures of marketed drugs and 150 generated structures. The generated structures have been collected from the generative experiments obtained within older platform versions (Chemistry42 v.1.22-1.25) and newer platform versions (generative experiments). The final consensus set consists of 111 molecular structures (50 drugs+61 generated structures) that were sampled unambiguously by the Insilico Medicine organic chemistry experts jury (see FIGS. 22A-22S). Those include 71 synthetically tractable molecular structures (ES class, mostly drug molecule structures) and 40 synthetically intractable molecular structures (HS class). The middle category with compounds possessing uncertainty in synthesis tractability was excluded from the final set as well as structures that were out of consensus. The composition of the final set is depicted on FIG. 13.

For the final set six scores were calculated based on the diverse SA modeling methods: ReRSA 1.0, ReRSA 3.0, SA Score, SYBA score, RAscore, SCScore (see FIGS. 22A-22S).

The key evaluation metric used for this experiment is Balanced Accuracy (BA) which is common to evaluate classification models. This metric was chosen for evaluation because it takes into account both sensitivity and specificity, making it suitable for problems with imbalanced classes. The BA is calculated using the following formula:

$$Balanced Accuracy = \frac{Sensitivity + Specificity}{2} ,$$

where Sensitivity represents the true positive rate (TPR):

$$Sensitivity = True Positive Rate ( TPR ) = \frac{TP}{TP + FN} ,$$

and Specificity represents the true negative rate (TNR):

$$Specificity = True Negative Rate ( TNR ) = \frac{TN}{TN + FP} .$$

To determine the best threshold for each score, we iterated over a range of potential thresholds and selected the one that maximized BA for the respective model (see Table 6).

TABLE 6
Optimal and default thresholds with corresponding BA for
the expert classification set for SA modeling approaches.

| | ReRSA | ReRSA | | | | | |
|---|---|---|---|---|---|---|---|
| | 3.0 | 3.0 | ReRSA | SA | RA | | SC |
| | STRICT | SOFT | 1.0 | Score | score | SYBA | Score |
| Optimal | 8.70 | 4.49 | 4.18 | 3.27 | 0.98 | 86.96 | 4.28 |
| threshold | | | | | | | |
| Scale | 1 ÷ 10 | 1 ÷ 10 | 1 ÷ 10 | 1 ÷ 10 | 0 ÷ 1 | (−∞; +∞) | 1 ÷ 5 |
| Best BA | 0.93 | 0.92 | 0.68 | 0.81 | 0.68 | 0.68 | 0.66 |
| Default | 6.5 | 6.5 | 6.5 | 6.0 | — | 0 | — |
| threshold | | | | | | | |
| BA | 0.92 | 0.65 | 0.51 | 0.51 | — | 0.63 | — |

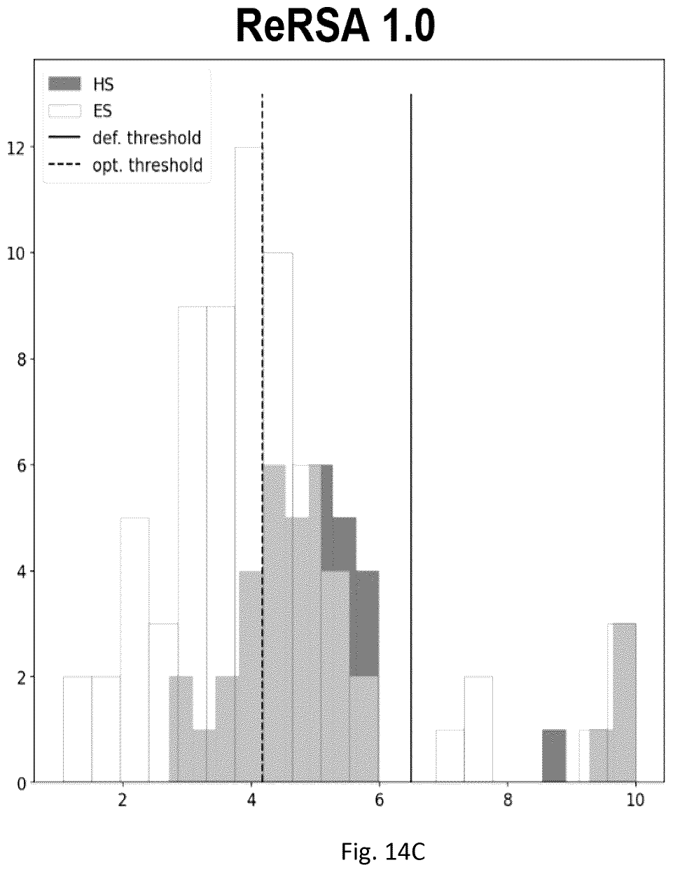

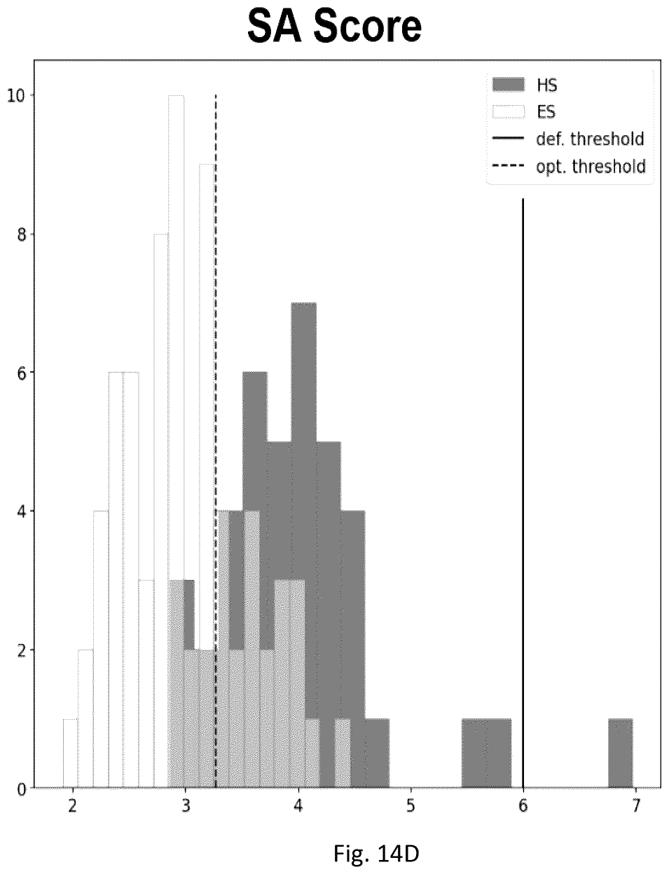

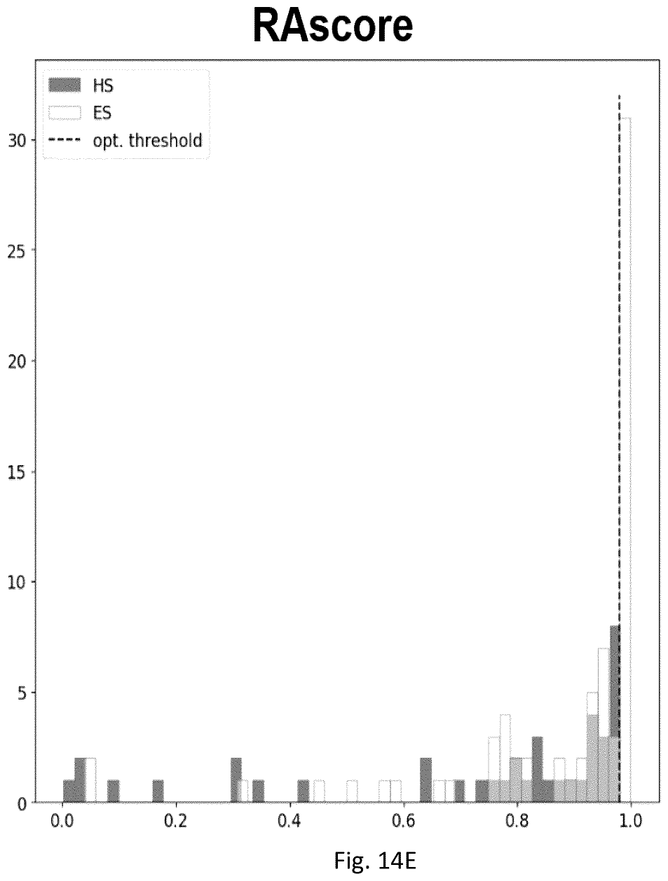

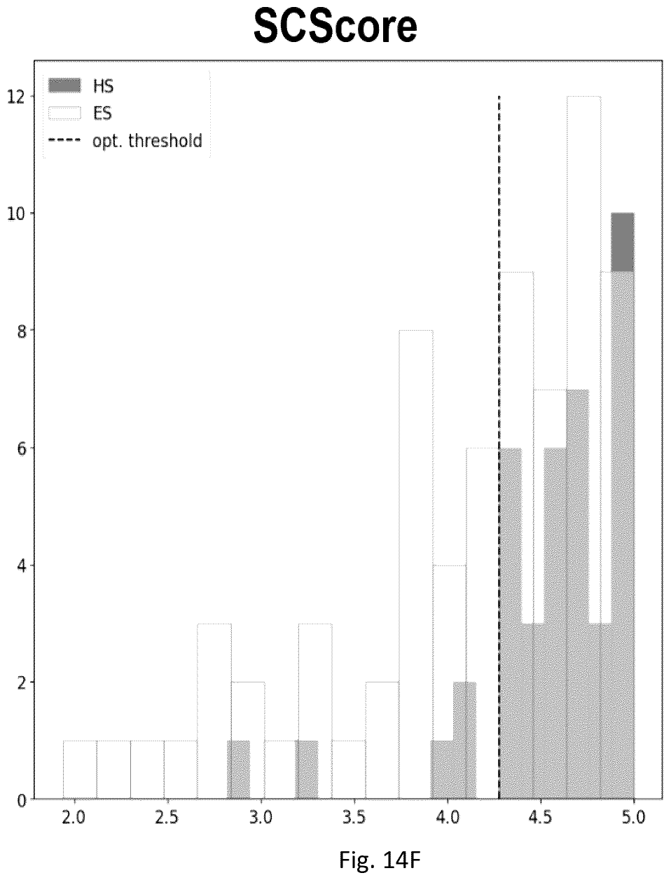

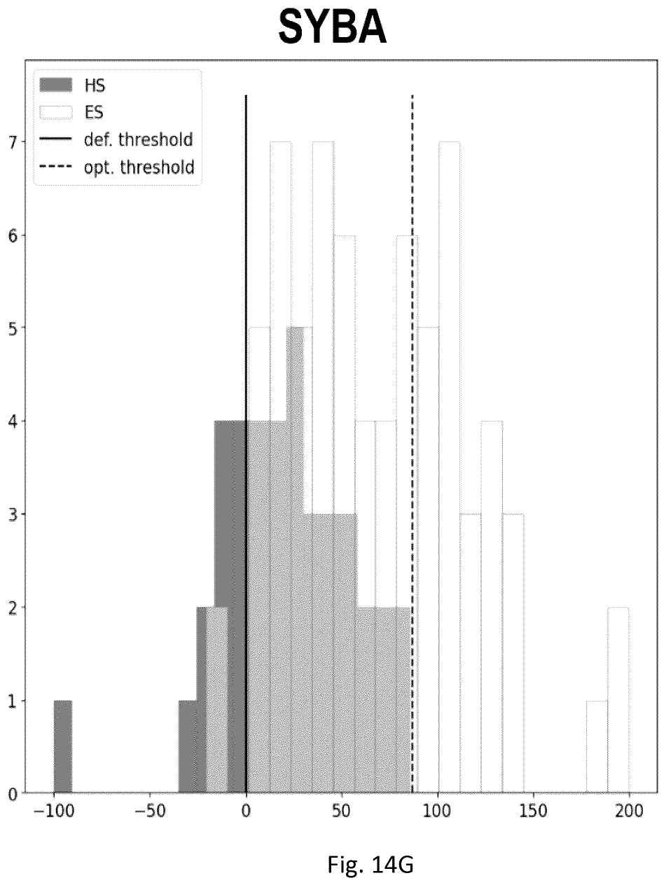

For each type of the SA models the threshold was utilized to make the transition from scale to classification possible. For certain SA models the authors provided the recommended threshold to separate accessible chemical space from inaccessible molecular structures. This was true for ReRSA (both versions 1.0 and 3.0 (described herein) have a recommended threshold of 6.5), SA Score (6.0) and SYBA (0). The recommended threshold was not provided by SCScore and RAscore authors, and that is why we have found the optimal (BA-wise) thresholds for both SA models based on our dataset. For SCScore it was 0.66 and for RAscore the optimal threshold was 0.68. To keep the consistency of optimal thresholds, the same were found for ReRSA versions (4.18 and 8.70 respectively), SA Score (3.27) and SYBA (86.96). It is important to mention that only for ReRSA 3.0 the transition from recommended to optimal threshold has not significantly altered the BA value, while for other SA modeling methods the BA value change was meaningful, especially for SA Score (0.51 vs 0.81). The best BA value (0.93) among SA models was observed for ReRSA 3.0 STRICT policy at optimal threshold. The second highest BA value (0.81) was shown by SA Score at its optimal threshold. The provided experiment clearly shows the classification power of the ReRSA 3.0 algorithm to discriminate between easy-to-synthesize and hard-to-synthesize molecular structures. The discrimination power of different SA modeling methods is visualized as the bar chart in FIGS. 14A-14G.

### Embodiments

In some embodiments, a computer-implemented method for determining the synthetic accessibility of a target molecular structure may involve accessing a molecular structures database comprising previously synthesized compounds. The method may include decomposing the molecular structures in the database into synthon-like fragments using a plurality of robust chemical reactions. Frequency statistics for each synthon-like fragment may be calculated based on their occurrence in the database, and these statistics may be stored in a fragments dictionary.

In some embodiments, the method may receive a target molecular structure represented as a chemical structure and decompose the target molecular structure into one or more splits, where each split comprises a set of synthon-like fragments generated by applying the robust chemical reactions. The synthon-like fragments may be converted into synthetic equivalents by applying conversion rules, and these equivalents may be indexed against a dataset of commercially available building blocks to determine building block availability.

In some embodiments, the method may further comprise calculating molecular descriptors for both the synthon-like fragments and the overall target molecular structure. A primary split score for each split may be determined based on the fragment frequency statistics and the calculated molecular descriptors. A final split score for each split may be calculated by factoring in the building block availability.

In some embodiments, the method may aggregate the final split scores to generate a synthetic accessibility score for the target molecular structure, and provide the synthetic accessibility score as an output.

In some embodiments, the plurality of robust chemical reactions used for decomposition may include at least 52 reactions, such as amine acylation, Buchwald-Hartwig amination, Suzuki coupling, Sonogashira coupling, reductive amination, Wittig reaction, SNAr substitution, Ugi reaction, the Michael reaction, metathesis, and combinations thereof.

In some embodiments, decomposing the target molecular structure into splits may comprise applying the plurality of robust chemical reactions to generate multiple retrosynthetic pathways, wherein each split corresponds to an independent branch of a retrosynthetic tree.

In some embodiments, the conversion rules applied to synthon-like fragments may generate multiple building block options for each fragment type, including variations such as different halide substitutions.

In some embodiments, calculating molecular descriptors may involve determining values for features such as ChiralCentersCount, RingCount, RingSideChainsCount, SpiroCount, BiggestRingSize, FusedRingsCount, BridgeAtomsCount, HeavyAtomCount, molecular weight, and normalized quadratic index.

In some embodiments, determining the primary split score may involve retrieving and transforming stored frequency statistics for each fragment, and combining them with contributions from the molecular descriptors. For fragments absent from the dictionary, frequency statistics may be estimated based on building block availability.

In some embodiments, calculating the final split score may involve normalizing the primary split score using an arctangent function and adjusting the score based on the number of fragments matched to commercially available building blocks.

In some embodiments, aggregating the split scores may involve selecting the best split with the lowest score, determining similarity between splits using a Tanimoto similarity metric, and combining weighted contributions from the splits to generate the synthetic accessibility score.

In some embodiments, the method may identify synthetically irrelevant substructures using SMARTS pattern matching, apply penalties to the synthetic accessibility score for such substructures, reward the use of multi-component reactions, and penalize macrocyclization reactions.

In some embodiments, the method may provide policy options, including a STRICT policy that filters molecular structures with rare or non-represented fragments, and a SOFT policy that applies penalties rather than filtering for underrepresented fragments or substructures.

In some embodiments, the STRICT policy may assign maximum penalty scores to molecular structures with detected irrelevant substructures, while the SOFT policy may apply a graduated penalty based on the degree of divergence from commonly represented fragments.

In some embodiments, the method may include a visualization step that highlights matching building blocks in the target molecular structure, displays commercial availability and CAS numbers, and links to chemical database entries for building blocks.

In some embodiments, the synthetic accessibility score may be scaled from 1 to 10, where lower scores correspond to structures with greater synthetic accessibility.

In some embodiments, a computer system may comprise a processor, memory, a molecular structures database, a fragments dictionary, and a building blocks database, all configured to implement the described method. The system may decompose target molecular structures, retrieve building block availability, calculate split and accessibility scores, and output results with visualizations.

In some embodiments, the processor-executable program instructions may decompose the target structure along multiple independent retrosynthetic pathways, apply diversity rewards for multiple pathways, and penalize the presence of irrelevant substructures as described.

In some embodiments, the building blocks database may aggregate data from multiple suppliers and may update availability and pricing in real time.

In some embodiments, the system may provide a user interface for entering molecular structures via SMILES or chemical drawing, displaying scores and confidence intervals, visualizing highlighted building blocks, and exporting results for use with other chemical informatics platforms.

In some embodiments, a non-transitory computer-readable storage medium may store instructions that, when executed, cause the processor to carry out all operations described above, including decomposition, scoring, aggregation, and reporting of synthetic accessibility.

In some embodiments, a method for determining retrosynthesis-related synthetic accessibility of a target molecular structure may be provided. The method may include accessing a molecular structures database containing a plurality of molecular structures. The method may further include decomposing the target molecular structure into a plurality of synthon-like fragments using a quasi-retrosynthetic decomposition procedure. The synthon-like fragments may be organized into a plurality of splits, with each split comprising a set of fragments resulting from an independent branch of a retrosynthetic tree. The method may include converting the synthon-like fragments into synthetic equivalents, indexing the synthetic equivalents against a dataset of commercially available starting materials, and calculating molecular descriptors for the synthon-like fragments. Fragment frequencies may be determined based on occurrence of the synthon-like fragments in a reference chemical space. The plurality of splits may be analyzed for diversity to reward molecular structures that possess multiple diverse synthetic pathways. Penalties may be applied for synthetically irrelevant substructures present in the target molecular structure. The method may further aggregate fragment scores, building block availability scores, molecular descriptor values, diversity scores, and penalty values to calculate an overall retrosynthesis-related synthetic accessibility score for the target molecular structure.

In some embodiments, the quasi-retrosynthetic decomposition procedure may comprise applying a plurality of robust chemical reactions, which may be encoded as SMARTS strings, to cleave the target molecular structure. Multiple retrosynthetic splits may be generated from the target molecular structure, and decomposition may continue until fragments cannot be further cleaved by available reactions.

In some embodiments, converting the synthon-like fragments into synthetic equivalents may include applying conversion rules to transform the fragments into purchasable compounds. The process may also involve generating alternative building block options comprising halide variations and validating the chemical structures and CAS numbers of the building blocks.

In some embodiments, calculating molecular descriptors may comprise determining one or more values including chiral centers count, ring count, ring side chains count, spiro count, biggest ring size, fused rings count, bridge atoms count, heavy atom count, molecular weight, and normalized quadratic index.

In some embodiments, determining fragment frequencies may include calculating the frequency of each synthon-like fragment as a ratio of molecules containing the fragment to the total number of molecules in a training dataset. A negative logarithm transformation may be applied to the frequency to generate the fragment score contribution, and higher score contributions may be assigned to rare fragments.

In some embodiments, analyzing the plurality of splits for diversity may comprise calculating similarity scores between splits using Tanimoto coefficients. Splits with lower similarity scores may be rewarded, while splits with higher similarity scores may be penalized.

In some embodiments, applying penalties may comprise identifying predetermined synthetically irrelevant substructures using SMARTS pattern matching. The method may also include penalizing macrocyclization reactions and rewarding multi-component reactions.

In some embodiments, the method may further comprise implementing a policy selection to balance novelty and synthetic accessibility. A STRICT policy may filter structures containing non-represented fragments, whereas a SOFT policy may apply penalties rather than filtration.

In some embodiments, a computer-implemented system for synthetic accessibility estimation may be provided. The system may comprise a processor configured to execute instructions, a memory storing a molecular structures database and a commercially available building blocks database, and a fragment analysis engine configured to decompose molecular structures into synthon-like fragments using retrosynthetic logic. The system may further include a building block assessment module configured to convert fragments into synthetic equivalents and index against commercially available starting materials, a scoring algorithm configured to calculate synthetic accessibility scores based on fragment frequencies, building block availability, molecular descriptors, pathway diversity, and structural penalties, and a visualization interface configured to display synthetic accessibility scores and building block information.

In some embodiments, the fragment analysis engine may be configured to apply over fifty robust chemical reaction patterns to decompose molecular structures. The engine may generate multiple independent splits representing different synthetic pathways and ensure bijective mapping such that the molecular structures can be reconstructed from the fragments.

In some embodiments, the building block assessment module may be configured to cross-reference fragments with vendor catalogs comprising ChemDiv and Enamine databases. The module may validate building block availability and pricing information and provide CAS numbers and supplier information for available building blocks.

In some embodiments, the scoring algorithm may be configured to normalize primary split scores using sigmoid functions. The scoring algorithm may calculate final split scores incorporating building block availability and aggregate split scores considering pathway diversity and structural complexity.

In some embodiments, the visualization interface may be configured to highlight unchangeable parts of building blocks found in target molecules. The interface may display CAS numbers and links to chemical database webpages and may further provide disclaimers for molecular parts not converted to commercially available building blocks.

In some embodiments, a method for creating a fragment dictionary for a synthetic accessibility prediction model may be provided. The method may comprise accessing a reference dataset of synthesized molecular structures and decomposing each molecular structure in the reference dataset into synthon-like fragments using quasi-retrosynthetic procedures. The fragments may be organized into splits representing independent synthetic pathways. The method may further comprise calculating frequencies of unique fragments across the reference dataset, computing molecular descriptors for each fragment, building a fragment dictionary associating each fragment with its frequency and descriptor values, and storing the fragment dictionary for use in synthetic accessibility scoring of target molecules.

In some embodiments, the reference dataset may comprise molecular structures from the PubChem database, the ChEMBL bioactivity database, the ZINC commercially available compounds database, and various vendor chemical catalogs.

In some embodiments, decomposing molecular structures may comprise applying BRICS-like fragmentation algorithms and utilizing robust reaction patterns comprising acylation, alkylation, coupling reactions, and heterocycle synthesis. The process may ensure that the resulting fragments represent valid molecular structures.

In some embodiments, calculating frequencies may comprise determining the occurrence count of each fragment across all molecules in the reference dataset. Frequencies may be normalized to values between zero and one, and logarithmic transformations may be applied to emphasize rare fragments.

In some embodiments, a method for prioritizing molecular structures for synthesis may be provided. The method may comprise receiving a plurality of candidate molecular structures from a generative chemistry platform, calculating retrosynthesis-related synthetic accessibility scores for each candidate molecular structure using fragment-based analysis, ranking the candidate molecular structures based on the synthetic accessibility scores, selecting molecular structures having the lowest synthetic accessibility scores for synthesis prioritization, and providing building block availability information for the selected molecular structures.

In some embodiments, the method may further comprise filtering candidate molecular structures based on policy selection. A STRICT policy may eliminate structures containing synthetically irrelevant substructures, whereas a SOFT policy may penalize rather than eliminate structures with novel substructures.

In some embodiments, calculating synthetic accessibility scores may comprise decomposing each candidate molecular structure into multiple synthetic pathway options, evaluating building block availability for each pathway, rewarding structures having diverse synthetic pathway options, and penalizing structures containing complex structural features.

One skilled in the art will appreciate that, for the processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In one embodiment, the present methods can include aspects performed on a computing system. As such, the computing system can include a memory device that has the computer-executable instructions for performing the methods. The computer-executable instructions can be part of a computer program product that includes one or more protocols or algorithms for performing any of the methods of any of the claims.

In one embodiment, any of the operations, processes, or methods, described herein can be performed or cause to be performed in response to execution of computer-readable instructions stored on a computer-readable medium and executable by one or more processors. The computer-readable instructions can be executed by a processor of a wide range of computing systems from desktop computing systems, portable computing systems, tablet computing systems, hand-held computing systems, as well as network elements, and/or any other computing device. The computer readable medium is not transitory. The computer readable medium is a physical medium having the computer-readable instructions stored therein so as to be physically readable from the physical medium by the computer/processor.

There are various vehicles by which processes and/or systems and/or other technologies described herein can be effected (e.g., hardware, software, and/or firmware), and that the preferred vehicle may vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The various operations described herein can be implemented, individually and/or collectively, by a wide range of hardware, software, firmware, or virtually any combination thereof. In one embodiment, several portions of the subject matter described herein may be implemented via application specific integrated circuits (ASICs), field programmable gate arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware are possible in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a physical signal bearing medium include, but are not limited to, the following: a recordable type medium such as a floppy disk, a hard disk drive (HDD), a compact disc (CD), a digital versatile disc (DVD), a digital tape, a computer memory, or any other physical medium that is not transitory or a transmission. Examples of physical media having computer-readable instructions omit transitory or transmission type media such as a digital and/or an analog communication medium (e.g., a fiber optic cable, a waveguide, a wired communication link, a wireless communication link, etc.).

It is common to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. A typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems, including feedback loops and control motors (e.g., feedback for sensing position and/or velocity; control motors for moving and/or adjusting components and/or quantities). A typical data processing system may be implemented utilizing any suitable commercially available components, such as those generally found in data computing/communication and/or network computing/communication systems.

The herein described subject matter sometimes illustrates different components contained within, or connected with, different other components. Such depicted architectures are merely exemplary, and that in fact, many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include, but are not limited to: physically mateable and/or physically interacting components and/or wirelessly interactable and/or wirelessly interacting components and/or logically interacting and/or logically interactable components.

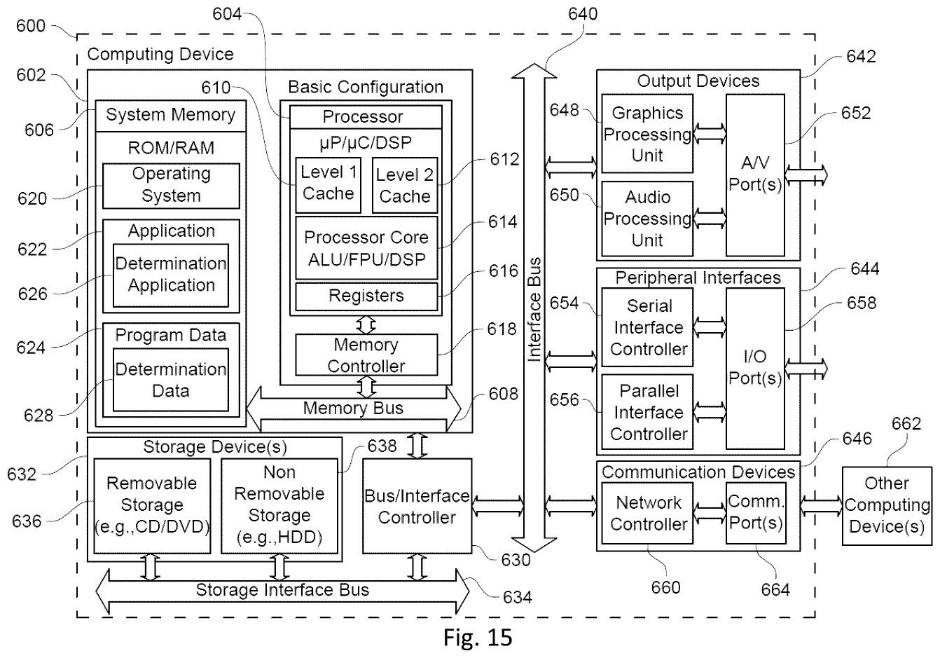

FIG. 15 shows an example computing device 600 (e.g., a computer) that may be arranged in some embodiments to perform the methods (or portions thereof) described herein. In a very basic configuration 602, computing device 600 generally includes one or more processors 604 and a system memory 606. A memory bus 608 may be used for communicating between processor 604 and system memory 606.

Depending on the desired configuration, processor 604 may be of any type including, but not limited to: a microprocessor (μP), a microcontroller (μC), a digital signal processor (DSP), or any combination thereof. Processor 604 may include one or more levels of caching, such as a level one cache 610 and a level two cache 612, a processor core 614, and registers 616. An example processor core 614 may include an arithmetic logic unit (ALU), a floating point unit (FPU), a digital signal processing core (DSP Core), or any combination thereof. An example memory controller 618 may also be used with processor 604, or in some implementations, memory controller 618 may be an internal part of processor 604.

Depending on the desired configuration, system memory 606 may be of any type including, but not limited to: volatile memory (such as RAM), non-volatile memory (such as ROM, flash memory, etc.), or any combination thereof. System memory 606 may include an operating system 620, one or more applications 622, and program data 624. Application 622 may include a determination application 626 that is arranged to perform the operations as described herein, including those described with respect to methods described herein. The determination application 626 can obtain data (e.g., determination data 628), such as pressure, flow rate, and/or temperature, and then determine a change to the system to change the pressure, flow rate, and/or temperature.

Computing device 600 may have additional features or functionality, and additional interfaces to facilitate communications between basic configuration 602 and any required devices and interfaces. For example, a bus/interface controller 630 may be used to facilitate communications between basic configuration 602 and one or more data storage devices 632 via a storage interface bus 634. Data storage devices 632 may be removable storage devices 636, non-removable storage devices 638, or a combination thereof. Examples of removable storage and non-removable storage devices include: magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include: volatile and non-volatile, removable and non-removable media implemented in any method or technology for storage of information, such as computer readable instructions, data structures, program modules, or other data.

System memory 606, removable storage devices 636 and non-removable storage devices 638 are examples of computer storage media. Computer storage media includes, but is not limited to: RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 600. Any such computer storage media may be part of computing device 600.

Computing device 600 may also include an interface bus 640 for facilitating communication from various interface devices (e.g., output devices 642, peripheral interfaces 644, and communication devices 646) to basic configuration 602 via bus/interface controller 630. Example output devices 642 include a graphics processing unit 648 and an audio processing unit 650, which may be configured to communicate to various external devices such as a display or speakers via one or more A/V ports 652. Example peripheral interfaces 644 include a serial interface controller 654 or a parallel interface controller 656, which may be configured to communicate with external devices such as input devices (e.g., keyboard, mouse, pen, voice input device, touch input device, etc.) or other peripheral devices (e.g., printer, scanner, etc.) via one or more I/O ports 658. An example communication device 646 includes a network controller 660, which may be arranged to facilitate communications with one or more other computing devices 662 over a network communication link via one or more communication ports 664.

The network communication link may be one example of a communication media. Communication media may generally be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR), and other wireless media. The term computer readable media as used herein may include both storage media and communication media.

Computing device 600 may be implemented as a portion of a small-form factor portable (or mobile) electronic device such as a cell phone, a personal data assistant (PDA), a personal media player device, a wireless web-watch device, a personal headset device, an application specific device, or a hybrid device that includes any of the above functions. Computing device 600 may also be implemented as a personal computer including both laptop computer and non-laptop computer configurations. The computing device 600 can also be any type of network computing device. The computing device 600 can also be an automated system as described herein.

The embodiments described herein may include the use of a special purpose or general-purpose computer including various computer hardware or software modules.

Embodiments within the scope of the present invention also include computer-readable media for carrying or having computer-executable instructions or data structures stored thereon. Such computer-readable media can be any available media that can be accessed by a general purpose or special purpose computer. By way of example, and not limitation, such computer-readable media can comprise RAM, ROM, EEPROM, CD-ROM or other optical disk storage, magnetic disk storage or other magnetic storage devices, or any other medium which can be used to carry or store desired program code means in the form of computer-executable instructions or data structures and which can be accessed by a general purpose or special purpose computer. When information is transferred or provided over a network or another communications connection (either hardwired, wireless, or a combination of hardwired or wireless) to a computer, the computer properly views the connection as a computer-readable medium. Thus, any such connection is properly termed a computer-readable medium. Combinations of the above should also be included within the scope of computer-readable media.

Computer-executable instructions comprise, for example, instructions and data which cause a general purpose computer, special purpose computer, or special purpose processing device to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

In some embodiments, a computer program product can include a non-transient, tangible memory device having computer-executable instructions that when executed by a processor, cause performance of a method that can include: providing a dataset having object data for an object and condition data for a condition; processing the object data of the dataset to obtain latent object data and latent object-condition data with an object encoder; processing the condition data of the dataset to obtain latent condition data and latent condition-object data with a condition encoder; processing the latent object data and the latent object-condition data to obtain generated object data with an object decoder; processing the latent condition data and latent condition-object data to obtain generated condition data with a condition decoder; comparing the latent object-condition data to the latent-condition data to determine a difference; processing the latent object data and latent condition data and one of the latent object-condition data or latent condition-object data with a discriminator to obtain a discriminator value; selecting a selected object from the generated object data based on the generated object data, generated condition data, and the difference between the latent object-condition data and latent condition-object data; and providing the selected object in a report with a recommendation for validation of a physical form of the object. The non-transient, tangible memory device may also have other executable instructions for any of the methods or method steps described herein. Also, the instructions may be instructions to perform a non-computing task, such as synthesis of a molecule and or an experimental protocol for validating the molecule. Other executable instructions may also be provided.

In some embodiments, a computer program product can include a non-transient, tangible memory device having computer-executable instructions that when executed by a processor, cause performance of a method described herein.

The non-transient, tangible memory device may also have other executable instructions for any of the methods or method steps described herein. Also, the instructions may be instructions to perform a non-computing task, such as synthesis of a molecule and or an experimental protocol for validating the molecule. Other executable instructions may also be provided.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

### Definitions

A split is a set of fragments and building blocks resulting from an independent branch of a retrosynthetic tree. One molecule can be splitted into multiple splits if many robust reactions can be applied to the starting molecule itself and to the intermediate fragments until the set of available reactions is exhausted.

A representative chemical space is a fraction of the chemical space that provides statistical clarity and the shape of normal distribution. The use of representative chemical space is extremely important since this assumption allows us not to explore the whole almost infinite chemical space. The best fit for that role are ready-to-use compound aggregators like open-sourced PubChem [53], ZINC [54] and ChEMBL [55,56] or vendor screening stocks like those from ChemDiv Inc [57]. and Enamine Inc. [58] or commercial datasets.

A robust reaction is an organic reaction or a group of similar reactions, or a general synthetic method which is widely used in the practice of organic synthesis of small drug molecules [59,60]. The list of sample reactions and resulting fragments and building blocks that can be exploited by ReRSA algorithm is given in FIGS. 16A-16H, but not limited to them. The tables in FIGS. 16A-16H illustrate ReRSA exploited robust reactions schemes.

A “synthon-like fragment” (later fragment) is a synthon-like part of a molecule that is produced after cheminformatics decomposition (virtual reaction) by a robust reaction. A fragment is not a real chemical structure, but possibly can be converted into a building block by cheminformatics transformation to become a real compound structure. The resulting building block can be checked for availability in building blocks vendor datasets.

A building block is a real (valid) chemical structure that can be found in the vendor datasets. Building blocks can be produced directly from some cheminformatics decompositions (virtual reactions) or by conversion from fragments (conversion rules). The resulting building blocks can be checked for availability in vendor datasets such as ChemDiv and Enamine. The availability of building blocks in the vendor datasets is considered as a factor of higher synthetic accessibility.

A policy selection is an option to balance between novelty and synthetic accessibility. Synthetic accessibility here is considered in the terms of actual presence of substructures/fragments in the reported chemical space. The STRICT policy optimizes synthetic accessibility within filtering structures that are either decomposed into non-represented fragments (see Algorithm 5) or contain non-represented or rare substructures (see Algorithm 4). Thus, STRICT policy ignores the potential novelty of a structure to be accessed. Oppositely, the SOFT policy allows penalization rather than filtration, when the ReRSA algorithm finds a structure containing underrepresented fragments/substructures.

All references recited herein are incorporated herein by specific reference in their entirety.
1. Vanhaelen Q., Lin Y.-C., Zhavoronkov A. The Advent of Generative Chemistry//ACS Med. Chem. Lett. ACS Publications, 2020. Vol. 11, No 8. P. 1496-1505.
2. Bilodeau C. et al. Generative models for molecular discovery: Recent advances and challenges//Wiley Interdiscip. Rev. Comput. Mol. Sci. Wiley, 2022. Vol. 12, No 5.
3. Gao W., Coley C. W. The Synthesizability of Molecules Proposed by Generative Models//J. Chem. Inf. Model. ACS Publications, 2020. Vol. 60, No 12. P. 5714-5723.
4. Baber J. C., Feher M. Predicting synthetic accessibility: application in drug discovery and development//Mini Rev. Med. Chem. europepmc.org, 2004. Vol. 4, No 6. P. 681-692.
5. Bonnet P. Is chemical synthetic accessibility computationally predictable for drug and lead-like molecules? A comparative assessment between medicinal and computational chemists//Eur. J. Med. Chem. Elsevier, 2012. Vol. 54. P. 679-689.
6. Zagribelnyy B. et al. Retrosynthesis-related synthetic accessibility: pat. 2021229454:A1 USA//World Patent. 2021.
7. Uspensky J. V. Introduction to mathematical probability//1937.
8. Corey E. J. General methods for the construction of complex molecules//J. Macromol. Sci. Part A Pure Appl. Chem. De Gruyter, 1967. Vol. 14, No 1. P. 19-38.
9. Corey E. J., Wipke W. T. Computer-Assisted Design of Complex Organic Syntheses//Science. 1969. Vol. 166, No 3902. P. 178-192.
10. Corey E. J., Long A. K., Rubenstein S. D. Computer-assisted analysis in organic synthesis//Science. science.org, 1985. Vol. 228, No 4698. P. 408-418.
11. Johnson A. P., Marshall C., Judson P. N. Starting material oriented retrosynthetic analysis in the LHASA program. 1. General description//J. Chem. Inf. Comput. Sci. American Chemical Society, 1992. Vol. 32, No 5. P. 411-417.
12. Gillet V. J. et al. SPROUT, HIPPO and CAESA: Tools for de novo structure generation and estimation of synthetic accessibility//Perspect. Drug Discov. Des. Springer, 1995. Vol. 3, No 1. P. 34-50.
13. Genheden S. et al. AiZynthFinder: a fast, robust and flexible open-source software for retrosynthetic planning//J. Cheminform. Springer, 2020. Vol. 12, No 1. P. 70.
14. AiZynthFinder: A tool for retrosynthetic planning. URL:github.com/MolecularAI/aizynthfinder (accessed: 10.05.2023).
15. ASKCOS: Software tools for organic synthesis]. URL:askcos.mit.edu/ (accessed: 09.05.2023).
16. CAS SciFindern—retrosynthesis software]. URL:cas.org/solutions/cas-scifinder-discovery-platform/cas-scifinder/synthesis-planning (accessed: 09.05.2023).
17. Reaxys]. URL:elsevier.com/solutions/reaxys (accessed: 13.05.2023).
18. Mikulak-Klucznik B. et al. Computational planning of the synthesis of complex natural products//Nature. nature.com, 2020. Vol. 588, No 7836. P. 83-88.
19. SYNTHIA™ Retrosynthesis Software]. URL:sigmaaldrich.com/AE/en/services/software-and-digital-platforms/synthia-retrosynthesis-software (accessed: 10.05.2023).
20. Thakkar A. et al. Retrosynthetic accessibility score (RAscore)—rapid machine learned synthesizability classification from AI driven retrosynthetic planning//Chem. Sci. Royal Society of Chemistry (RSC), 2021. Vol. 12, No 9. P. 3339-3349.
21. Bertz S. H. The first general index of molecular complexity//J. Am. Chem. Soc. American Chemical Society, 1981. Vol. 103, No 12. P. 3599-3601.
22. Whitlock H. W. On the Structure of Total Synthesis of Complex Natural Products//J. Org. Chem. American Chemical Society, 1998. Vol. 63, No 22. P. 7982-7989.
23. Rucker G., Rucker C. Walk counts, labyrinthicity, and complexity of acyclic and cyclic graphs and molecules/Journal of chemical information and. ACS Publications, 2000.
24. Barone R., Chanon M. A new and simple approach to chemical complexity. Application to the synthesis of natural products//J. Chem. Inf. Comput. Sci. ACS Publications, 2001. Vol. 41, No 2. P. 269-272.
25. Huang Q., Li L.-L., Yang S.-Y. RASA: a rapid retrosynthesis-based scoring method for the assessment of synthetic accessibility of drug-like molecules//J. Chem. Inf. Model. ACS Publications, 2011. Vol. 51, No 10. P. 2768-2777.
26. Coley C. W. Defining and Exploring Chemical Spaces//Trends in Chemistry. Elsevier, 2021. Vol. 3, No 2. P. 133-145.
27. Mercado R., Kearnes S. M., Coley C. W. Data Sharing in Chemistry: Lessons Learned and a Case for Mandating Structured Reaction Data//J. Chem. Inf. Model. 2023. Vol. 63, No 14. P. 4253-4265.
28. Takaoka Y. et al. Development of a method for evaluating drug-likeness and ease of synthesis using a data set in which compounds are assigned scores based on chemists' intuition//J. Chem. Inf. Comput. Sci. ACS Publications, 2003. Vol. 43, No 4. P. 1269-1275.
29. Sheridan R. P. et al. Modeling a Crowdsourced Definition of Molecular Complexity//J. Chem. Inf. Model. American Chemical Society, 2014. Vol. 54, No 6. P. 1604-1616.
30. Ertl P., Schuffenhauer A. Estimation of synthetic accessibility score of drug-like molecules based on molecular complexity and fragment contributions//J. Cheminform. Springer, 2009. Vol. 1, No 1. P. 8.
31. Podolyan Y., Walters M. A., Karypis G. Assessing synthetic accessibility of chemical compounds using machine learning methods//J. Chem. Inf. Model. ACS Publications, 2010. Vol. 50, No 6. P. 979-991.
32. Fukunishi Y. et al. Prediction of synthetic accessibility based on commercially available compound databases//J. Chem. Inf. Model. ACS Publications, 2014. Vol. 54, No 12. P. 3259-3267.
33. Coley C. W. et al. SCScore: Synthetic Complexity Learned from a Reaction Corpus//J. Chem. Inf. Model. ACS Publications, 2018. Vol. 58, No 2. P. 252-261.
34. Vorsilik M. et al. SYBA: Bayesian estimation of synthetic accessibility of organic compounds//J. Cheminform. jcheminfbiomedcentral.com, 2020. Vol. 12, No 1. P. 35.
35. Chen S., Jung Y. Estimating the synthetic accessibility of molecules with building block and reaction-aware SAScore//J. Cheminform. Springer Science and Business Media LLC, 2024. Vol. 16, No 1.
36. Weininger D. SMILES, a chemical language and information system. 1. Introduction to methodology and encoding rules//J. Chem. Inf. Comput. Sci. American Chemical Society, 1988. Vol. 28, No 1. P. 31-36.
37. Wang S. et al. DeepSA: a deep-learning driven predictor of compound synthesis accessibility//J. Cheminform. 2023. Vol. 15, No 1. P. 103.
38. Yu J. et al. Organic Compound Synthetic Accessibility Prediction Based on the Graph Attention Mechanism//J. Chem. Inf. Model. ACS Publications, 2022. Vol. 62, No 12. P. 2973-2986.
39. Liu C.-H. et al. RetroGNN: Fast Estimation of Synthesizability for Virtual Screening and De Novo Design by Learning from Slow Retrosynthesis Software//J. Chem. Inf. Model. ACS Publications, 2022. Vol. 62, No 10. P. 2293-2300.
40. Kim H. et al. DFRscore: Deep Learning-Based Scoring of Synthetic Complexity with Drug-Focused Retrosynthetic Analysis for High-Throughput Virtual Screening//J. Chem. Inf. Model. ACS Publications, 2023.
41. Neeser R. M., Correia B., Schwaller P. FSscore: A Machine Learning-based Synthetic Feasibility Score Leveraging Human Expertise//arXiv [cs.LG]. 2023.
42. Chapelle O., Schölkopf B., Zien A. Semi-supervised Learning. MIT Press, 2006. 508 p.
43. van Tilborg D., Alenicheva A., Grisoni F. Exposing the Limitations of Molecular Machine Learning with Activity Cliffs//J. Chem. Inf. Model. 2022. Vol. 62, No 23. P. 5938-5951.
44. Fourches D., Ash J. 4D-quantitative structure-activity relationship modeling: making a comeback//Expert Opin. Drug Discov. Taylor & Francis, 2019. Vol. 14, No 12. P. 1227-1235.
45. Karniadakis G. E. et al. Physics-informed machine learning//Nature Reviews Physics. 2021. Vol. 3, No 6. P. 422-440.
46. Lajiness M. S., Maggiora G. M., Shanmugasundaram V. Assessment of the consistency of medicinal chemists in reviewing sets of compounds//J. Med. Chem. ACS Publications, 2004. Vol. 47, No 20. P. 4891-4896.
47. Rdkit: Open-Source Cheminformatics Tool]. URL:Rdkit.org (accessed: 19.05.2023).
48. PubChem. PubChem]. URL:pubchem.ncbi.nlm.nih.gov/ (accessed: 23.02.2024).
49. Kochev N. et al. Computational prediction of synthetic accessibility of organic molecules with Ambit-synthetic accessibility tool//Org. Chem. An Indian J. 2018. Vol. 14. P. 123.
50. Li B., Chen H. Prediction of Compound Synthesis Accessibility Based on Reaction Knowledge Graph//Molecules. mdpi.com, 2022. Vol. 27, No 3.
51. Parrot M. et al. Integrating synthetic accessibility with AI-based generative drug design//J. Cheminform. 2023. Vol. 15, No 1. P. 83.
52. Degen J. et al. On the art of compiling and using “drug-like” chemical fragment spaces//ChemMedChem. Wiley, 2008. Vol. 3, No 10. P. 1503-1507.
53. Kim S. et al. PubChem 2023 update//Nucleic Acids Res. 2023. Vol. 51, No D1. P. D1373-D1380.
54. Irwin J. J. et al. ZINC20—A Free Ultralarge-Scale Chemical Database for Ligand Discovery//J. Chem. Inf. Model. American Chemical Society, 2020. Vol. 60, No 12. P. 6065-6073.
55. Gaulton A. et al. ChEMBL: a large-scale bioactivity database for drug discovery//Nucleic Acids Res. academic.oup.com, 2012. Vol. 40, No Database issue. P. D1100-D1107.
56. Mendez D. et al. ChEMBL: towards direct deposition of bioassay data//Nucleic Acids Res. Oxford University Press (OUP), 2019. Vol. 47, No D1. P. D930-D940.
57. Chemdiv inc—fully integrated target-to-clinic contract research organization]. URL:chemdiv.com/ (accessed: 19.05.2023).
58. EnamineStore]. URL:new.enaminestore.com/ (accessed: 21.05.2023).
59. Schneider N. et al. Big Data from Pharmaceutical Patents: A Computational Analysis of Medicinal Chemists' Bread and Butter//J. Med. Chem. 2016. Vol. 59, No 9. P. 4385-4402.
60. Brown D. G., Boström J. Analysis of Past and Present Synthetic Methodologies on Medicinal Chemistry: Where Have All the New Reactions Gone?//J. Med. Chem. 2016. Vol. 59, No 10. P. 4443-4458.
61. Balaban A. T. Chemical graphs//Theor. Chim. Acta. Springer Nature, 1979. Vol. 53, No 4. P. 355-375.
62. SMARTS™—A Language for Describing Molecular Patterns]//Daylight Chemical Information Systems, Inc. 2007. URL:daylight.com/dayhtml/doc/theory/theory.smarts.html.
63. Ivanenkov Y. A. et al. Chemistry42: An AI-Driven Platform for Molecular Design and Optimization//J. Chem. Inf. Model. 2023. Vol. 63, No 3. P. 695-701.
64. Schwall K., Zielenbach K. SciFinder a new generation of research tool//Chemical innovation. pascal-francis.inist.fr, 2000.
65. Oprea T. I., Bologa C. Molecular Complexity: You Know It When You See It//J. Med. Chem. 2023. Vol. 66, No 18. P. 12710-12714.
66. Hoffmann T., Gastreich M. The next level in chemical space navigation: going far beyond enumerable compound libraries//Drug Discov. Today. 2019. Vol. 24, No 5. P. 1148-1156.
67. Beckers M., Fechner N., Stiefl N. 25 Years of Small-Molecule Optimization at Novartis: A Retrospective Analysis of Chemical Series Evolution//J. Chem. Inf. Model. ACS Publications, 2022. Vol. 62, No 23. P. 6002-6021.
68. Ivanenkov Y. et al. The Hitchhiker's Guide to Deep Learning Driven Generative Chemistry//ACS Med. Chem. Lett. ACS Publications, 2023. Vol. 14, No 7. P. 901-915.
69. Boda K., Seidel T., Gasteiger J. Structure and reaction based evaluation of synthetic accessibility//J. Comput. Aided Mol. Des. Springer, 2007. Vol. 21, No 6. P. 311-325.

1. A method for training a model to estimate synthetic accessibility, comprising:
providing a retrosynthesis-related synthetic accessibility model;
accessing a molecular structures database;
obtaining at least one molecular structure from the molecular structures database with the model;
virtually slicing the at least one molecular structure into synthon-like fragments with the model;
determining a frequency of the synthon-like fragments in natural molecules with the model;
calculating molecular descriptors for the synthon-like fragments with the model;
determining an aggregated synthetic accessibility score for the synthon-like fragments with the model; and
storing the aggregated synthetic accessibility score for the synthon-like fragments in a database for the model.

2. The method of claim 1, comprising wherein a subscore of each synthon-like fragment is calculated with the model as a function taking into account descriptors and fragment frequency in a dictionary over the whole training dataset.

3. The method of claim 2, wherein the subscore represents a chemical complexity of the synthon-like fragment, including:
number of chiral carbon atom,
total number of rings,
number of side chains attached to the ring systems,
number of spiro carbon atoms,
number of atoms in the largest ring of molecular structure if it is bigger than 6, otherwise 0,
number of fused rings in a molecular structure,
number of bridgehead atoms in the bicyclic pattern(s) of molecular structure,
number of atoms other than hydrogen,
molecular weight value, and
normalized quadratic index 1 calculated as (3−2·A+Z1/2).

4. The method of claim 3, further comprising:
performing a canonicalization for normalizing a chemical line notation of the molecular structure, where the valence, charge, and aromaticity is checked;
inputting the molecular structure if the valence, charge, and aromaticity are valid; and
removing the molecular structure if the valence, charge, and/or aromaticity are invalid.

5. The method of claim 4, further comprising:
performing a decomposition on the molecular structure into a set of splits;
checking available of fragment in a fragment dictionary that has the fragments and frequency thereof;
obtaining primary split score from all subscores;
obtaining transformed split score with a mathematic transformation;
obtaining final split score from the transformed split score taking into account number of fragments that are commercially available building blocks, where more commercially available building blocks for the fragments has a lower final split score; and
aggregating final split scores for the fragments taking into account differences between a split with lower final split score and other final split scores of other splits to obtain a retrosynthesis-related synthetic accessibility model score.

6. The method of claim 5, further comprising:
smoothing retrosynthesis-related synthetic accessibility model scores for a plurality of molecular structures to avoid sharp drops in scores over a chemical space of the plurality of molecular structures.

7. The method of claim 1, further comprising training the model with the retrosynthesis-related synthetic accessibility model scores for a plurality of molecular structures.

8. A method of estimating synthetic accessibility, comprising:
inputting a target molecule into a synthetic accessibility system having a retrosynthesis-related synthetic accessibility model;
decomposing the target molecule into molecular synthon-like fragments;
converting the synthon-like fragments into their synthetic equivalents;
indexing the synthetic equivalents in the dataset of commercially available starting materials;
providing penalties for synthetically irrelevant unprecedented substructures;
calculating a synthetic accessibility score components for the molecular fragments for the target molecular structure;
determining a sum of synthetic accessibility score components for the fragments; and
providing the synthetic accessibility score and building blocks visualization for the target molecule.

9. The method of claim 8, further comprising determining frequency of synthon-like fragments by:
obtaining a retrosynthetic decomposition of the molecular structure of the target molecule to obtain synthon-like fragments thereof;
organizing the synthon-like fragments into sets of fragments;
aggregating sets of fragments into a homogenous dataset of synthon-like fragments;
calculating frequencies of the synthon-like fragments in a reported chemical space, such as natural or non-synthesized molecules;
obtaining a dataset of the synthon-like fragments associated with their respective calculated frequencies; and
identifying synthon-like fragments with higher frequencies.

10. The method of claim 9, further comprising:
determining a synthetic accessibility of the molecular structure where synthon-like fragments with higher frequencies have a higher contribution to the synthetic accessibility; and
providing the synthetic accessibility for a synthetic route of the target molecule.

11. The method of claim 8, further comprising performing a target molecular structure assessment for synthetic accessibility by:
obtaining a retrosynthetic decomposition of the molecular structure of the target molecule to obtain synthon-like fragments thereof;
organizing the synthon-like fragments into sets of fragments that are synthetic equivalents;
indexing the synthetic equivalent sets of fragments;
identifying commercially available starting materials for the synthetic equivalent sets of fragments; and
obtaining a dataset of the indexed synthetic equivalent sets of fragments associated with the commercially available starting materials, wherein when more synthetic equivalents are found in the dataset the higher contribution to the final estimate of synthetic accessibility is obtained for the molecular structure.

12. The method of claim 8, further comprising:
taking into account the context of molecular complexity for synthon-like fragments, wherein the molecular structure is described with structural descriptors; and
storing descriptor values for each synthon-like fragment in a database for the retrosynthesis-related synthetic accessibility model.

13. The method of claim 8, further comprising filtering molecular structures by:
analyzing fragments of synthon-like fragments in the set of fragments; and
rewarding fragments that possessing multiple synthetic routes, wherein more diverse sets of fragments with higher number of synthetic routes have a higher contribution to determined synthetic accessibility.

14. The method of claim 8, further comprising analyzing a synthetic route of a target molecule by:
obtaining a virtual reaction for a reaction step in the synthetic route;
analyzing the virtual reaction for influence on synthetic accessibility of the target molecule;
rewarding multi-component reaction steps;
penalizing macrocyclization reactions;
obtaining a predetermined synthetically irrelevant substructures inadequate for a reaction step;
penalizing a synthesis route with the predetermined synthetically irrelevant substructures; and
determining synthetic accessibility for the target molecule after the rewards and/or penalties.

15. The method of claim 1, further comprising calculating an overall retrosynthesis-related synthetic accessibility score by:
obtaining score of frequencies of synthon-like fragments;
obtaining score of building blocks of target molecule;
obtaining score of structural descriptor values;
obtaining score of diversity and/or similarity score of sets of fragments;
obtaining score of rewards and/or penalties for reaction step of synthetic route;
obtaining score of penalty for synthetically irrelevant synthon-like fragment or portion thereof; and
aggregating the score of each of the foregoing to obtain a final synthetic accessibility score.

16. The method of claim 1, further comprising:
identifying a first target molecule with a lower synthetic accessibility score compared to a second target molecule;
selecting the first target molecule for synthesis; and
obtaining the synthetic route with the lower synthetic accessibility score.

17. The method of claim 16, further comprising:
synthesizing the target molecule to obtain a real, physical form of the target molecule.

18. The method of claim 16, further comprising obtaining real, physical forms of starting reagents for the synthetic route of the first targe molecule.

19. One or more non-transitory computer readable media storing instructions that in response to being executed by one or more processors, cause a computer system to perform operations, the operations comprising the method of claim 1.

20. A computer system comprising:
one or more processors; and
one or more non-transitory computer readable media storing instructions that in response to being executed by the one or more processors, cause the computer system to perform operations, the operations comprising the method of claim 1.

21. One or more non-transitory computer readable media storing instructions that in response to being executed by one or more processors, cause a computer system to perform operations, the operations comprising the method of claim 8.

22. A computer system comprising:
one or more processors; and
one or more non-transitory computer readable media storing instructions that in response to being executed by the one or more processors, cause the computer system to perform operations, the operations comprising the method of claim 8.
